SEQUENCE LISTING (4 sequences)

SEQ ID NO: 1 - RNA, 21 nt, Artificial Sequence
  Synthetic Oligonucleotide
aacucaauaaagugcuuugaa

SEQ ID NO: 2 - RNA, 21 nt, Artificial Sequence
  Synthetic Oligonucleotide
  misc_feature (1)..(6): Each residue has 2′-O-methyl (2′-OMe) ribo- sugar modification
  misc_feature (7)..(7): Residue has 2-deoxy-2-fluoro (2′-F) ribo- sugar modification
  misc_feature (8)..(8): Residue has 2′-O-methyl (2′-OMe) ribo-sugar modification
  misc_feature (9)..(11): Each residue has 2-deoxy-2-fluoro (2′-F) ribo- sugar modification
  misc_feature (12)..(21): Each residue has 2′-O-methyl (2′-OMe) ribo- sugar modification
  misc_feature (19)..(20): Phosphorothioate linkage between residues 19 and 20
  misc_feature (20)..(21): Phosphorothioate linkage between residues 20 and 21
aacucaauaaagugcuuugaa

SEQ ID NO: 3 - RNA, 23 nt, Artificial Sequence
  Synthetic Oligonucleotide
uucaaagcacuuuauugaguuuc

SEQ ID NO: 4 - RNA, 23 nt, Artificial Sequence
  Synthetic Oligonucleotide
  misc_feature (1)..(2): Phosphorothioate linkage between residues 1 and 2
  misc_feature (1)..(1): Residue has a 2′-O-methyl (2′-OMe) ribo-sugar modification
  misc_feature (2)..(3): Phosphorothioate linkage between residues 2 and 3
  misc_feature (2)..(2): Residue has 2-deoxy-2-fluoro (2′-F) ribo-sugar modification
  misc_feature (3)..(5): Each residue has 2′-O-methyl (2′-OMe) ribo- sugar modification
  misc_feature (7)..(7): Residue has 2′-O-methyl (2′-OMe) ribo-sugar modification
  misc_feature (8)..(9): Each residue has 2-deoxy-2-fluoro (2′-F) ribo- sugar modification
  misc_feature (10)..(13): Each residue has 2′-O-methyl (2′-OMe) ribo- sugar modification
  misc_feature (14)..(14): Residue has 2-deoxy-2-fluoro (2′-F) ribo-sugar modification
  misc_feature (15)..(15): Residue has 2′-O-methyl (2′-OMe) ribo-sugar modification
  misc_feature (16)..(16): Residue has 2-deoxy-2-fluoro (2′-F) ribo-sugar modification
  misc_feature (17)..(22): Each residue has 2′-O-methyl (2′-OMe) ribo- sugar modification
uucaaagcacuuuauugaguuuc

(19) United States
(12) Patent Application Publication
(10) Pub. No.: US 2022/0175934 A1
(43) Pub. Date: Jun. 9, 2022

(54) MULTIVALENT LIGAND CLUSTERS FOR TARGETED DELIVERY OF THERAPEUTIC AGENTS

(71) Applicant: Mitotherapeutix LLC, Cheshire, CT (US)

(72) Inventor: Pengcheng Patrick SHAO, Warrington, PA (US)

(21) Appl. No.: 17/300,676

(22) PCT Filed: Mar. 19, 2020
(86) PCT No.: PCT/US2020/023603

§ 371 Date: Sep. 23, 2021

Related U.S. Application Data
(60) Provisional application No. 62/952,607, filed on Dec. 23, 2019.
(60) Provisional application No. 62/821,628, filed on Mar. 21, 2019.

(51) Int. Cl. A61K 47/54; C07H 21/02; C12N 15/11

(52) U.S. Cl. CPC A61K 47/549; C12N 2320/32; C12N 15/111; C07H 21/02

(57) ABSTRACT

Targeting ligand clusters, and methods of preparing same, are described. A targeting ligand cluster may include first linkers attached to phenolic hydroxyl groups of gallic acid, and one or more targeting ligands attached to each of the first linkers. The targeting ligand cluster may also include a second linker attached to a carboxylic acid of the gallic acid, and at least one of a protecting group, a phosphoramidite, or an oligonucleotide attached to the second linker.

### RELATED APPLICATIONS

This application claims benefit under 35 U.S.C. § 119(e) of U.S. Provisional application Ser. No. 62/821,628 filed Mar. 21, 2019 and U.S. Provisional application Ser. No. 62/952,607 filed Dec. 23, 2019, the disclosure of each which is incorporated by reference herein in its entirety.

### FIELD OF THE INVENTION

The invention relates, in part, to compositions and methods of their use in therapeutic molecule delivery.

### BACKGROUND

Oligonucleotides are a class of compound with high molecular weight and polyanionic nature. They generally have very low cell membrane permeability. Thus, target ligands are often conjugated to oligonucleotide compounds to enhance in vivo delivery tissue specificity and cell uptake. In some cases, multivalent ligand clusters have advantage over single ligands in enhancing delivery to targeted tissues. For example, a multivalent N-Acetylgalactosamine (GalNAc) ligand cluster has significantly higher binding affinity to asialoglycoprotein receptor (ASGPR) than individual GalNAc ligands and, thus, higher efficiency in delivering therapeutic oligonucleotides into liver. ASGPR is expressed, significantly, in hepatocytes and can mediate efficient uptake through receptor endocytosis. N-Acetylgalactosamine ligand and ligand clusters can facilitate delivery of oligonucleotide drugs into hepatocytes.

### SUMMARY OF THE INVENTION

According to an aspect of the invention, a compound is provided that includes a targeting ligand cluster of Formula 2

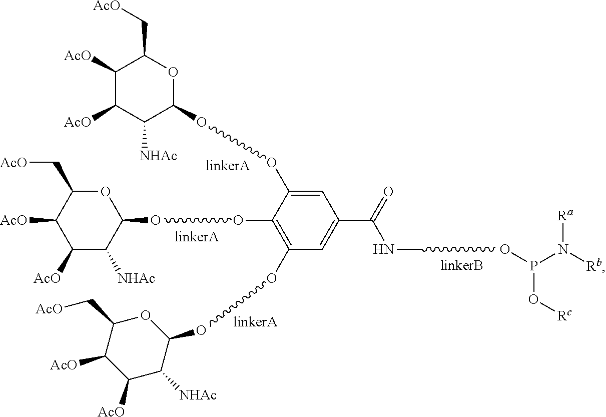

wherein linkerA is independently selected and comprises at least one spacer, with one end of linkerA attaching to a GalNAc targeting ligand and the other end attaching to a phenolic hydroxy group of gallic acid through an ether bond; wherein linkerB is independently selected and comprises at least one spacer, with one end of linkerB attaching to a phosphorous atom of a phosphoramidite or an oligonucleotide and the other end attaching to the carboxylic acid of gallic acid through an amide bond; wherein R^{a} comprises a C1 to C6 alkyl, C3 to C6 cycloalkyl, an isopropyl group, or R^{a} is joined with R^{b} through a nitrogen atom to form a cycle; wherein R^{b} comprises a C1 to C6 alkyl, C3 to C6 cycloalkyl, an isopropyl group, or R^{b} is joined with R^{a} through a nitrogen atom to form a cycle; and wherein R^{C} comprises a phosphite and phosphate protecting group, or a 2-cyanoethyl group. In some embodiments, the independently selected linkerA includes at least one of polyethylene glycol (PEG), an alkyl group, a cycloalkyl group, an alkenyl group, a cycloalkenyl group, an alkynyl group, an aryl group, an aralkyl group, an aralkenyl group, and an aralkynyl group. In some embodiments, the independently selected linkerA includes one or more heteroatoms, aliphatic heterocycles, heteroaryls, amino acids, nucleotides, and saccharides. In certain embodiments, the independently selected linkerB includes at least one of PEG, an alkyl group, a cycloalkyl group, an alkenyl group, a cycloalkenyl group, an alkynyl group, an aryl group, an aralkyl group, an aralkenyl group, and an aralkynyl group. In some embodiments, the independently selected linkerB includes one or more heteroatoms, aliphatic heterocycles, heteroaryls, amino acids, nucleotides, and saccharides. In some embodiments, the phosphate protecting group includes at least one of methyl, allyl, 2-cyanoethyl, 4-cyano-2-butenyl, 2-cyano-1,1-dimethylethyl, 2-(trimethylsilyl)ethyl, 2-(S-acetylthio)ethyl, 2-(S-pivaloylthio)ethyl, 2-(4-nitrophenyl)ethyl, 2,2,2-trichloroethyl, 2,2,2-trichloro-1,1-dimethylethyl, 1,1,1,3,3,3-hexafluoro-2-propyl, fluorenyl-9-methyl, 2-chlorophenyl, 4-chlorophenyl, and 2,4-dichlorophenyl. In some embodiments, the independently selected linkerA includes one or more of:

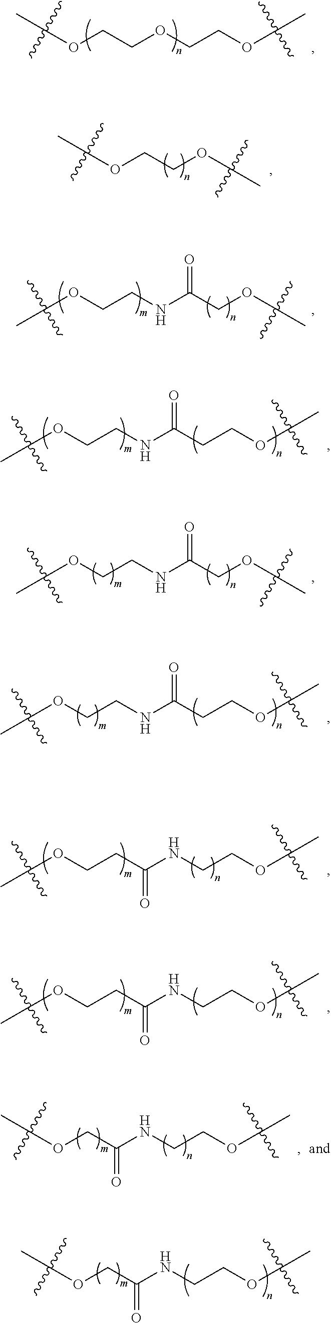

wherein m is an integral number 1, 2, 3, 4, 5, 6, 7, 8, 9, 10, 11, or 12; and n is an integral number 1, 2, 3, 4, 5, 6, 7, 8, 9, 10, 11, or 12. In certain embodiments, the independently selected linkerB includes one or more of:

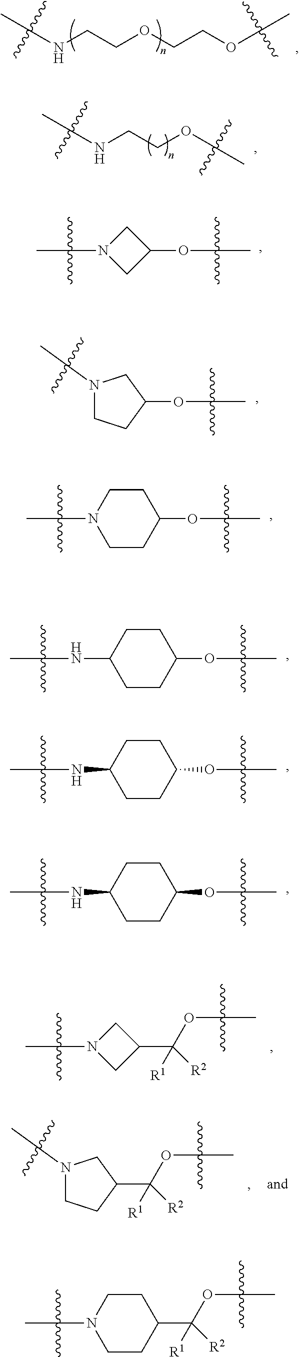

wherein n is an integral number 1, 2, 3, 4, 5, 6, 7, 8, 9, 10, 11, or 12; wherein R¹ comprises H, methyl (Me), ethyl (Et), cyclopropyl, or R¹ is joined with R² through a carbon atom to form a 3-6 member ring; and wherein R² comprises H, Me, Et, cyclopropyl, or R² is joined with R¹ through a carbon atom to form a 3-6 member ring. In some embodiments, the independently selected linkerB includes one or more of:

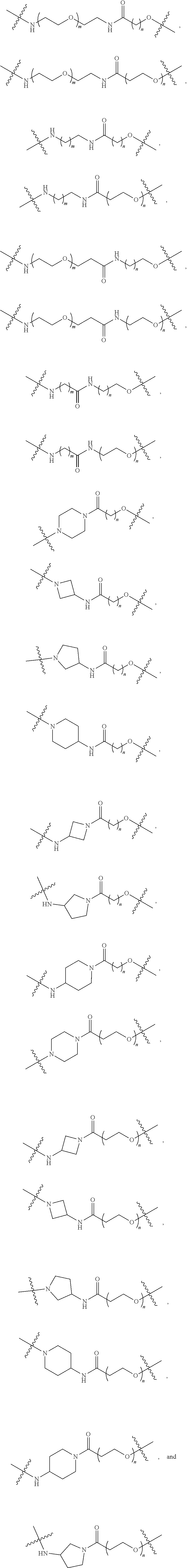

wherein m is an integral number 1, 2, 3, 4, 5, 6, 7, 8, 9, 10, 11, or 12; and n is an integral number 1, 2, 3, 4, 5, 6, 7, 8, 9, 10, 11, or 12. In certain embodiments, the targeting ligand cluster includes one of Ligands A-I. In some embodiments, the targeting ligand cluster includes one of Ligands J-WW. In some embodiments, the targeting ligand cluster includes a Gallic acid and at least one of the independently selected LinkerA comprises polyethylene glycol (PEG) directly bonded to the oxygen of a hydroxyl group of the Gallic acid. In certain embodiments, the targeting ligand cluster also includes an oligonucleotide attached to the targeting ligand cluster thereby forming a targeting ligand cluster/nucleic acid complex. In some embodiments, the targeting ligand cluster/nucleic acid complex is MITO-A, MITO-B, MITO-C, MITO-D, MITO-E, MITO-F, MITO-G, MITO-H, or MITO-I.

According to another aspect of the invention, a composition is provided that includes any embodiment of an aforementioned compound. In some embodiments, the composition further includes a pharmaceutically acceptable carrier.

According to another aspect of the invention a composition is provided that includes any embodiment of an aforementioned targeting ligand cluster. In certain embodiments, the composition further includes a pharmaceutically acceptable carrier.

According to another aspect of the invention, a compound is provided that includes the structure of Formula 3:

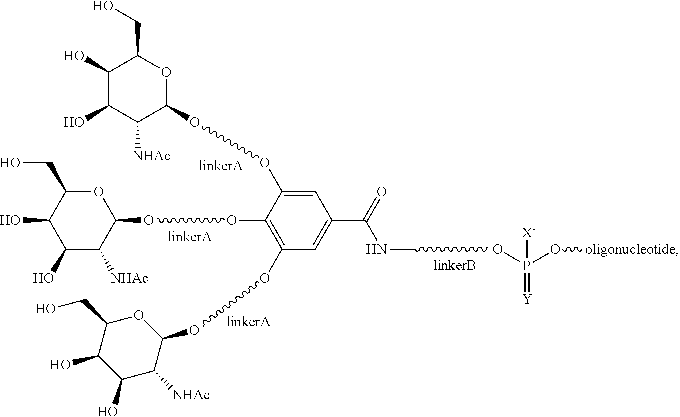

wherein X is at least one of oxygen (O) and sulfur (S); wherein Y is at least one of O, S, and NH; wherein linkerA is independently selected and comprises at least one spacer, with one end of linkerA attaching to a GalNAc targeting ligand and the other end attaching to a phenolic hydroxy group of gallic acid through an ether bond; wherein linkerB is independently selected and comprises at least one spacer, with one end of linkerB attaching to a phosphorous atom of a phosphoramidite or an oligonucleotide and the other end attaching to the carboxylic acid of gallic acid through an amide bond. In some embodiments, the oligonucleotide includes at least one of a small interfering RNA (siRNA), a single strand siRNA, a microRNA (miRNA), an antisense oligonucleotide, a messenger RNA (mRNA), a ribozyme, a plasmid, an immune stimulating nucleic acid, an antagomir, and an aptamer. In certain embodiments, the independently selected linkerA includes at least one of PEG, an alkyl group, a cycloalkyl group, an alkenyl group, a cycloalkenyl group, an alkynyl group, an aryl group, an aralkyl group, an aralkenyl group, and an aralkynyl group. In some embodiments, the independently selected linkerA includes one or more heteroatoms, aliphatic heterocycles, heteroaryls, amino acids, nucleotides, and saccharides. In some embodiments, the independently selected linkerB includes at least one of PEG, an alkyl group, a cycloalkyl group, an alkenyl group, a cycloalkenyl group, an alkynyl group, an aryl group, an aralkyl group, an aralkenyl group, and an aralkynyl group. In some embodiments, the independently selected linkerB includes one or more heteroatoms, aliphatic heterocycles, heteroaryls, amino acids, nucleotides, and saccharides. In certain embodiments, the independently selected linkerA includes one or more of:

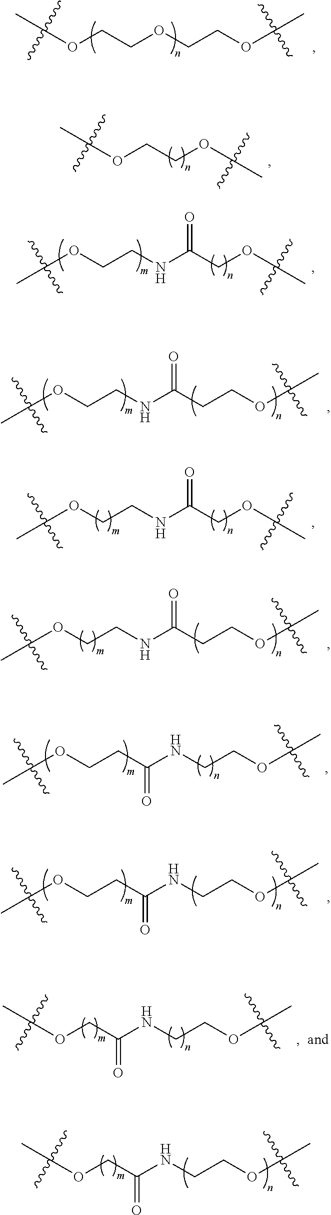

wherein m is an integral number 1, 2, 3, 4, 5, 6, 7, 8, 9, 10, 11, or 12; and n is an integral number 1, 2, 3, 4, 5, 6, 7, 8, 9, 10, 11, or 12. In some embodiments, the independently selected linkerB includes one or more of:

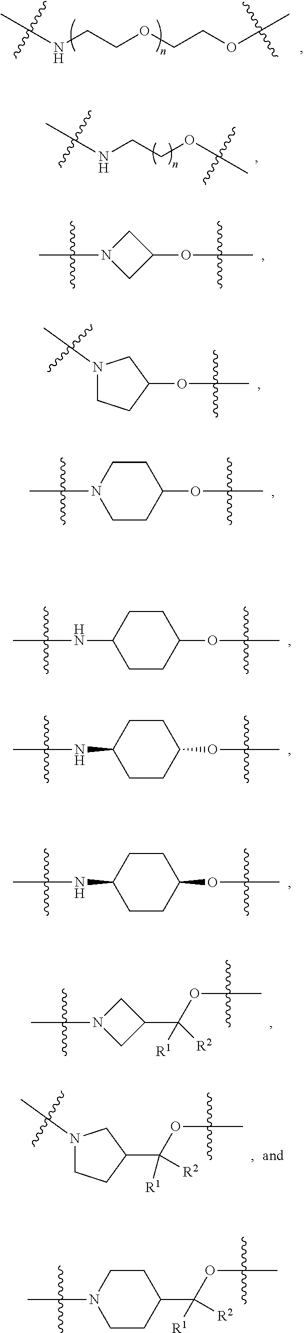

wherein n is an integral number 1, 2, 3, 4, 5, 6, 7, 8, 9, 10, 11, or 12; wherein R¹ comprises H, Me, Et, cyclopropyl, or R¹ is joined with R² through a carbon atom to form a 3-6 member ring; and wherein R² comprises H, Me, Et, cyclopropyl, or R² is joined with R¹ through a carbon atom to form a 3-6 member ring. In some embodiments, the independently selected linkerB includes one or more of:

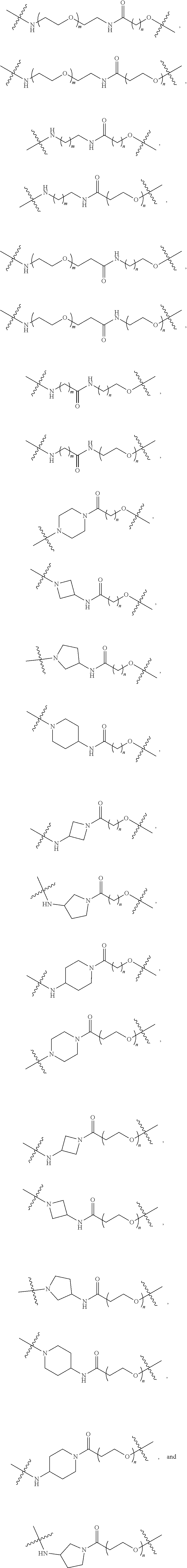

wherein m is an integral number 1, 2, 3, 4, 5, 6, 7, 8, 9, 10, 11, or 12; and n is an integral number 1, 2, 3, 4, 5, 6, 7, 8, 9, 10, 11, or 12. In certain embodiments, the targeting ligand cluster includes one of Ligands A-I. In some embodiments, the targeting ligand cluster includes one of Ligands J-WW. In some embodiments, the targeting ligand cluster includes a Gallic acid and at least one of the independently selected LinkerA comprises polyethylene glycol (PEG) directly bonded to the oxygen of a hydroxyl group of the Gallic acid. In some embodiments, the compound is MITO-A, MITO-B, MITO-C, MITO-D, MITO-E, MITO-F, MITO-G, MITO-H, or MITO-I.

According to another aspect of the invention, a composition is provided that includes any embodiment of an aforementioned compound. In some embodiments, the composition further includes a pharmaceutically acceptable carrier.

According to another aspect of the invention, a compound is provide that includes a targeting ligand cluster of Formula 1:

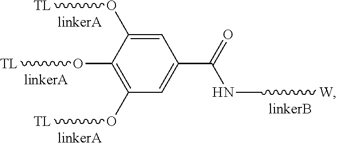

wherein TL is one or more targeting ligands, including but not limited to: N-acetylgalactosamine, galactose, galactosamine, N-formyl-galactosamine, N-propionylgalactosamine, N-n-butanoylgalactosamine, and N-iso-butanoylgalactosamine; wherein one or more TLs may be different from one or more other TLs of the same targeting ligand cluster; wherein linkerA is independently selected and comprises one or more bifunctional spacers, with one end of linkerA attaching to the targeting ligand and the other end attaching to a phenolic hydroxy group of gallic acid through an ether bond; wherein linkerB is independently selected and comprises a bifunctional spacer, with one end of linkerB attaching to a phosphoramidite or an oligonucleotide and the other end attaching to the carboxylic acid of gallic acid through an amide bond; and wherein W is H, a protecting group, phosphoramidite or oligonucleotide. In certain embodiments, the targeting ligand cluster includes one or more of Ligands A-I. In some embodiments, the targeting ligand cluster includes one or more of Ligands J-WW. In some embodiments, the targeting ligand cluster includes a Gallic acid; and at least one of the independently selected linkerA comprises polyethylene glycol (PEG) directly bonded to the oxygen of a hydroxyl group of the Gallic acid. In some embodiments, the targeting ligand cluster also includes an oligonucleotide attached to the targeting ligand cluster thereby forming a targeting ligand cluster/nucleic acid complex. In certain embodiments, the targeting ligand cluster/nucleic acid complex includes a compound set forth as MITO-A, MITO-B, MITO-C, MITO-D, MITO-E, MITO-F, MITO-G, MITO-H, or MITO-I.

According to another aspect of the invention, a composition is provided that includes any embodiment of an aforementioned compound. In some embodiments, the composition further includes a pharmaceutically acceptable carrier.

According to another aspect of the invention a composition is provided that includes any embodiment of an aforementioned targeting ligand cluster. In certain embodiments, the composition further includes a pharmaceutically acceptable carrier.

According to another aspect of the invention, a targeting ligand cluster is provided that includes a structure motif derived from Gallic acid; a linker off each hydroxyl group of the Gallic acid; and a linker off the amide group of the Gallic acid, wherein at least one of the linkers comprises polyethylene glycol (PEG) directly bonded to the oxygen of a hydroxyl group of the Gallic acid. In some embodiments, the targeting cluster also includes an oligonucleotide attached to the targeting ligand cluster thereby forming a targeting ligand cluster/nucleic acid complex. In some embodiments, the targeting ligand cluster includes a compound set forth as one of Ligands A-I. In certain embodiments, the targeting ligand cluster includes a compound set forth as one of Ligands J-WW.

According to another aspect of the invention, a targeting ligand cluster is provided that includes one or more independently selected first linkers each attached to a phenolic hydroxyl group of gallic acid; one or more independently selected targeting ligands attached to each of the first linkers; a second linker attached to a carboxylic acid of the gallic acid; and at least one of a protecting group and a phosphoramidite attached to the second linker. In some embodiments, the first linkers are attached to the phenolic hydroxyl groups through ether bonds. In some embodiments, the one or more targeting ligands include at least one of N-acetylgalactosamine, galactose, galactosamine, N-formyl-galactosamine, N-propionylgalactosamine, N-n-butanoylgalactosamine, and N-iso-butanoylgalactosamine. In some embodiments, the second linker is attached to a carboxylic acid through an amide bond. In certain embodiments, the first linkers include at least one of polyethylene glycol (PEG), an alkyl group, a cycloalkyl group, an alkenyl group, a cycloalkenyl group, an alkynyl group, an aryl group, an aralkyl group, an aralkenyl group, an aralkynyl group, one or more heteroatoms, one or more aliphatic heterocycles, one or more heteroaryls, one or more amino acids, one or more nucleotides, and one or more saccharides. In some embodiments, the second linker includes at least one of polyethylene glycol (PEG), an alkyl group, a cycloalkyl group, an alkenyl group, a cycloalkenyl group, an alkynyl group, an aryl group, an aralkyl group, an aralkenyl group, an aralkynyl group, one or more heteroatoms, one or more aliphatic heterocycles, one or more heteroaryls, one or more amino acids, one or more nucleotides, and one or more saccharides. In some embodiments, the three first linkers are each attached to a different phenolic hydroxyl group of gallic acid. In some embodiments, the targeting ligand cluster includes at least one of Ligands A-I. In certain embodiments, the targeting ligand cluster includes at least one of Ligands J-WW. In some embodiments, the targeting ligand cluster also includes an oligonucleotide attached to the targeting ligand cluster thereby forming a targeting ligand cluster/nucleic acid complex. In some embodiments, the targeting ligand cluster/nucleic acid complex includes a compound set forth as MITO-A, MITO-B, MITO-C, MITO-D, MITO-E, MITO-F, MITO-G, MITO-H, or MITO-I.

According to another aspect of the invention, a composition is provided that includes any embodiment of an aforementioned targeting ligand cluster. In certain embodiments, the composition further includes a pharmaceutically acceptable carrier.

According to another aspect of the invention a method of preparing a targeting ligand cluster is provided, the method including: performing an esterification reaction on gallic acid to produce a first compound comprising a tert-Butylester of gallic acid; performing an SN2 reaction or an Mitsunobu reaction to attach linkerA on phenolic hydroxy groups of gallic acid ester to produce a second compound; performing a glycosylation reaction on a second compound to produce a third compound; performing a deprotection reaction on the third compound to produce a fourth compound; performing an amide coupling reaction on the fourth compound to produce a fifth compound; and performing a phosphorylation reaction on the fifth compound. In some embodiments, the method also includes attaching a nucleic acid molecule to the targeting ligand cluster thereby forming a ligand cluster/nucleic acid complex. In certain embodiments, the ligand cluster/nucleic acid complex includes a compound set forth as MITO-A, MITO-B, MITO-C, MITO-D, MITO-E, MITO-F, MITO-G, MITO-H, or MITO-I.

According to another aspect of the invention, a targeting ligand cluster/nucleic acid complex is provided, the targeting ligand cluster/nucleic acid complex including: a) a targeting ligand cluster comprising one or more independently selected first linkers each attached to a phenolic hydroxyl group of gallic acid; b) one or more independently selected targeting ligands attached to each of the first linkers; c) a second linker attached to a carboxylic acid of the gallic acid; and d) at least one of a protecting group and a phosphoramidite attached to the second linker; wherein the targeting ligand cluster is attached to a nucleic acid forming a targeting ligand cluster/nucleic acid complex. In some embodiments, there are three first linkers each attached to a different phenolic hydroxyl group of the gallic acid. In some embodiments, there is more than one independently selected first linker and each of the one or more is the same as the other first linkers. In certain embodiments, two or three of the first linkers are different from the other first linkers. In some embodiments, the nucleic acid includes an RNA molecule, optionally an siRNA molecule. In some embodiments, the targeting ligand cluster/nucleic acid complex includes a compound set forth as MITO-A, MITO-B, MITO-C, MITO-D, MITO-E, MITO-F, MITO-G, MITO-H, or MITO-I.

According to another aspect of the invention a compound set forth as any one of Ligands A-I is provided.

According to another aspect of the invention, a composition is provided that includes one or more of Ligand A-I. In certain embodiments the composition also includes a pharmaceutically acceptable carrier.

According to another aspect of the invention, a composition is provided that includes one or more of Ligand J-WW. In some embodiments the composition also includes a pharmaceutically acceptable carrier.

According to another aspect of the invention, a composition is provided that includes an embodiment of any aforementioned targeting ligand cluster, wherein the targeting ligand cluster is conjugated to an siRNA. In certain embodiments the composition also includes a pharmaceutically acceptable carrier. In some embodiments, the targeting ligand cluster includes one of Ligands A-I. In some embodiments, the targeting ligand cluster includes one of Ligands J-WW. In some embodiments, the targeting ligand cluster conjugated to the siRNA includes one of MITO-A, MITO-B, MITO-C, MITO-D, MITO-E, MITO-F, MITO-G, MITO-H, and MITO-I.

According to another aspect of the invention, a method of reducing expression of a target gene in a cell is provided, the method including contacting a cell capable of expressing the target gene with an embodiment of any aforementioned targeting ligand cluster wherein the targeting ligand cluster includes an siRNA that reduces expression of the target gene. In certain embodiments, the cell is a liver cell, a heart cell, a kidney cell, an immune system cell, a muscle cell, or a neuronal cell. In some embodiments, the cell is an in vitro cell. In some embodiments, the cell is an in vivo cell. In certain embodiments, the cell is in a subject. In some embodiments, the subject is a human. In some embodiments, the contacting includes administering the composition to the subject. In certain embodiments, the expression of the target gene in the cell and/or subject is associated with a disease or condition and reducing expression of the target gene treats the disease or condition.

According to another aspect of the invention, a compound set forth as one of MITO-A, MITO-B, MITO-C, MITO-D, MITO-E, MITO-F, MITO-G, MITO-H, or MITO-I is provided.

According to another aspect of the invention, a composition is provided that includes one or more of MITO-A, MITO-B, MITO-C, MITO-D, MITO-E, MITO-F, MITO-G, MITO-H, and MITO-I and also includes a pharmaceutically acceptable carrier.

### BRIEF DESCRIPTION OF THE DRAWINGS

FIG. 1 provides embodiments of targeting ligand clusters set forth as Ligands A-WW. Embodiments of structures of Mito GalNAc phosphoramidite are shown in targeting ligand clusters identified as: Ligand A-Ligand I.

FIG. 2 shows sequences and sequence modifications used in certain studies. Sense strands shown are: AACUCAAUAAAGUGCUUUGAA (SEQ ID NO: 1) and L*aacucaAuAAAgugcuuug*a*a (SEQ ID NO: 2). Antisense strands shown are: UUCAAAGCACUUUAUUGAGUUUC(SEQ ID NO: 3) and u*U*caaAgCAcuuuAuUgaguu*u*c (SEQ ID NO: 4). In the sequences, the lower case letters indicate 2′-MeO nucleotide; the upper case letters indicate 2′-F nucleotide; the asterisks (*) indicate phosphorothioate; and “L” indicates the target ligand.

FIG. 3 provides a bar graph showing percentage of remaining FXII in plasma in the Mito-GalNAc conjugated siRNA treatment groups normalized to the PBS treated group. The graph shows the percent of remaining FXII in plasma at three time points: 5 days, 14 days, and 30 days after administration of the GalNaC conjugated siRNA treatment. Nine GalNAc conjugated siRNAs administered were: Mito-A, Mito-B, Mito-C, Mito-D, Mito-E, Mito-F, Mito-G, Mito-H, and Mito-I and data from Day 5 (left bar), Day 14 (center bar), and Day 30 (right bar) is shown for each.

### DETAILED DESCRIPTION

The present disclosure provides compounds that use gallic acid as a scaffold for delivering oligonucleotide agents, including but not limited to siRNAs. The present disclosure also provides methods of making and using compounds that use gallic acid as a scaffold and can be conjugated to an agent of interest and facilitate delivery of the agent of interest into a cell. In some embodiments of the invention a targeting ligand cluster is prepared and linked to a nucleic acid agent (or other agent of interest). As used herein, the term “targeting ligand cluster/nucleic acid complex” means a targeting ligand cluster of the invention that is linked to a nucleic acid, a non-limiting example of which is an siRNA. In some aspects of the invention, a targeting ligand cluster is prepared as set forth herein, linked to one or more nucleic acid agents thus forming a targeting ligand cluster/nucleic acid complex, the complex is contacted with a cell, and the one or more nucleic acid agents are delivered into the contacted cell. As used herein the terms “targeting ligand cluster” and “ligand cluster” may be used interchangeably. The invention in part includes compounds having a structure motif derived from Gallic acid, which is also referred to herein as compounds that use Gallic acid as a scaffold. Certain embodiments of such compounds of the invention can be linked to one or more agents of interest and used to deliver the agent(s) of interest into a cell and/or subject. In some embodiments, therapeutic agents are delivered to cells and/or subjects using embodiments of compositions and methods of the invention.

### Definitions

Unless specified otherwise, the following terms have the following meanings:

“Conjugate” or “conjugate group” means an atom or group of atoms bound to an oligonucleotide or other oligomer. In general, conjugate groups modify one or more properties of the compound to which they are attached, including, but not limited to pharmacodynamics, pharmacokinetic, binding, absorption, cellular distribution, cellular uptake, charge, and/or clearance properties.

“Linked” when referring to the connection between two molecules means that two molecules are joined, directly or indirectly, by a covalent bond or that two molecules are associated via noncovalent bonds (e.g., hydrogen bonds or ionic bonds). An example of a Compound A being directly joined to a Compound B may be represented as A-B. An example of a Compound A being indirectly joined to a Compound B may be represented as A-C-B, where Compound A is indirectly joined to Compound B through Compound C. It will be appreciated that more than one intermediary compound may be presented in situations of indirect joining of compounds. In some embodiments, where the term “linked” refers to the association between two molecules via noncovalent bonds, the association between the two different molecules has a Kn of less than 1×10⁻⁴ M (e.g., less than 1×10⁻⁵ M, less than 1×10⁻⁶ M, or less than 1×10⁻⁷ M) in physiologically acceptable buffer (e.g., phosphate buffered saline).

“Nucleic acid” refers to molecules composed of monomeric nucleotides. A nucleic acid includes ribonucleic acids (RNA), deoxyribonucleic acids (DNA), single-stranded nucleic acids (ssDNA), double-stranded nucleic acids (dsDNA), small interfering ribonucleic acids (siRNA) and microRNAs (miRNA). A nucleic acid may also comprise any combination of these elements in a single molecule. A nucleic acid may include natural nucleic acids, non-natural nucleic acids, or a combination of natural and non-natural nucleic acids. A nucleic acid may also be referred to herein as a nucleotide sequence, or as a polynucleotide.

An “oligomer” is a nucleotide sequence containing up to 5, up to 10, up to 15, up to 20, or more than 20 nucleotides or nucleotide base pairs. In some embodiments, an oligomer has a nucleobase sequence that is at least partially complementary to a coding sequence in an expressed target nucleic acid or target gene within a cell. In some embodiments, the oligomers, upon delivery to a cell expressing a gene, are able to inhibit the expression of the underlying gene. The gene expression can be inhibited in vitro or in vivo. Non-limiting examples of oligomers that may be included in methods and complexes of the invention are: oligonucleotides, single-stranded oligonucleotides, single-stranded antisense oligonucleotides, short interfering RNAs (siRNAs), single-stranded siRNA, double-strand RNAs (dsRNA), micro RNAs (miRNAs), short hairpin RNAs (shRNA), ribozymes, interfering RNA molecules, a dicer substrate, an antisense oligonucleotide, a messenger RNA (mRNA), a ribozyme, a plasmid, an immune stimulating nucleic acid, an antagomir, and an aptamer.

“Oligonucleotide” means a polymer of linked nucleotides each of which can be independently modified or unmodified.

“Single-stranded oligonucleotide” means a single-stranded oligomer and in certain embodiments of the invention a single-stranded oligonucleotide may comprise a sequence at least partially complementary to a target mRNA, that is capable of hybridizing to a target mRNA through hydrogen bonding under mammalian physiological conditions (or comparable conditions in vitro). In some embodiments, a single-stranded oligonucleotide is a single stranded antisense oligonucleotide.

“siRNA” is a short interfering RNA or silencing RNA. siRNAs are a class of double-stranded RNA molecules, that may be 20-25 (or shorter) base pairs in length, similar to microRNA (miRNA) that operate within the RNA interference (RNAi) pathway. siRNAs interferes with the expression of specific genes with complementary nucleotide sequences to the siRNA by degrading mRNA after transcription, preventing translation. siRNAs act in cells to silence gene expression by inducing the RNA-induced silencing complex (RISC) to cleave messenger RNA (mRNA).

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5.sup.th Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987.

Unless otherwise stated, structures depicted herein are also meant to include compounds that differ only in the presence of one or more isotopically enriched atoms. For example, compounds having the depicted structures that differ only in the replacement of hydrogen by deuterium or tritium, or the replacement of a carbon by ¹³C or ¹⁴C are within the scope of this invention. Such compounds may be useful, for example, as analytical tools, as probes in biological assays, or as therapeutic agents in accordance with the present invention.

In a formula, is a single bond where the stereochemistry of the moieties immediately attached thereto is not specified, is absent or a single bond, and or is a single or double bond.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

The terms “purified,” “substantially purified,” and “isolated” refer to a compound useful in the present invention being free of other, dissimilar compounds with which the compound is normally associated in its natural state, so that the compound comprises at least 0.5%, 1%, 5%, 10%, 20%, 50%, 75%, 80%, 85%, 90%, 95%, 96%, 97%, 98%, 99%, 99.5%, 99.9% of the mass, by weight, of a given sample or composition. In one embodiment, these terms refer to the compound comprising at least 95%, 98%, 99%, or 99.9% of the mass, by weight, of a given sample or composition.

The term “aliphatic” includes both saturated and unsaturated, nonaromatic, straight chain (e.g., unbranched), branched, acyclic, and cyclic (e.g., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, the term “alkyl” includes straight, branched and cyclic alkyl groups. An analogous convention applies to other generic terms such as “alkenyl”, “alkynyl”, and the like. Furthermore, the terms “alkyl”, “alkenyl”, “alkynyl”, and the like encompass both substituted and unsubstituted groups. In certain embodiments, “aliphatic” is used to indicate those aliphatic groups (cyclic, acyclic, substituted, unsubstituted, branched or unbranched) having 1-20 carbon atoms. Aliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkyl” refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-20 carbon atoms. In another embodiment, the alkyl group employed contains 1-15 carbon atoms. In another embodiment, the alkyl group employed contains 1-10 carbon atoms. In another embodiment, the alkyl group employed contains 1-8 carbon atoms. In another embodiment, the alkyl group employed contains 1-5 carbon atoms. Examples of alkyl radicals include, but are not limited to, methyl (e.g., unsubstituted methyl (Me)), ethyl (e.g., unsubstituted ethyl (Et)), propyl (e.g., unsubstituted propyl (Pr)), n-propyl, isopropyl, butyl (e.g., unsubstituted butyl (Bu)), n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substitutents. Alkyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkenyl” denotes a monovalent group derived from a straight- or branched-chain hydrocarbon moiety having at least one carbon-carbon double bond by the removal of a single hydrogen atom. In certain embodiments, the alkenyl group employed in the invention contains 2-20 carbon atoms. In some embodiments, the alkenyl group employed in the invention contains 2-15 carbon atoms. In another embodiment, the alkenyl group employed contains 2-10 carbon atoms. In still other embodiments, the alkenyl group contains 2-8 carbon atoms. In yet other embodiments, the alkenyl group contains 2-5 carbons. Alkenyl groups include, for example, ethenyl, propenyl, butenyl, 1-methyl-2-buten-1-yl, and the like, which may bear one or more substituents. Alkenyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “alkynyl” refers to a monovalent group derived from a straight- or branched-chain hydrocarbon having at least one carbon-carbon triple bond by the removal of a single hydrogen atom. In certain embodiments, the alkynyl group employed in the invention contains 2-20 carbon atoms. In some embodiments, the alkynyl group employed in the invention contains 2-15 carbon atoms. In another embodiment, the alkynyl group employed contains 2-10 carbon atoms. In still other embodiments, the alkynyl group contains 2-8 carbon atoms. In still other embodiments, the alkynyl group contains 2-5 carbon atoms. Representative alkynyl groups include, but are not limited to, ethynyl, 2-propynyl (propargyl), 1-propynyl, and the like, which may bear one or more substituents. Alkynyl group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted). Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃^{+X-}, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{a}, —C(═O)N(R^{bb})₂, —OC(═O)N(R^{bb})₂, —R^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{a}, —SO₂N(R^{bb})₂, —SO₂R^{a}, —SO₂OR^{aa}, —OSO₂R^{aa}, —S(═O)R^{a}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R.sup.dd groups; wherein X⁻ is a counterion; or two geminal hydrogens on a carbon atom are replaced with the group ═O, .═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of e is, independently, selected from hydrogen, —OH, —OR^{aa}, —N(R^{cc})₂, —CN, —C(═O)R^{aa}, —C(═O)N(R^{cc})₂, —CO₂R′, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻is a counterion; each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{CC} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(RE)₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion; each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂₀C₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R⁹⁹ substituents can be joined to form ═O or ═S; wherein X⁻ is a counterion.

The term “amino” refers to a group of the formula (—NH₂). A “substituted amino” refers either to a mono-substituted amine (—NHR^{h}) of a disubstituted amine (—NR^{h}₂), wherein the R^{h} substituent is any substituent as described herein that results in the formation of a stable moiety (e.g., a suitable amino protecting group; aliphatic, alkyl, alkenyl, alkynyl, heteroaliphatic, heterocyclic, aryl, heteroaryl, acyl, amino, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted). In certain embodiments, the R^{h} substituents of the disubstituted amino group (—NR^{h}₂) form a 5- to 6-membered heterocyclic ring.

The term “alkoxy” refers to a “substituted hydroxyl” of the formula (—OR), wherein Rⁱ is an optionally substituted alkyl group as defined herein, and the oxygen moiety is directly attached to the parent molecule.

The term “alkylthioxy” refers to a “substituted thiol” of the formula (—SR^{r}), wherein R is an optionally substituted alkyl group as defined herein, and the sulfur moiety is directly attached to the parent molecule.

The term “alkylamino” refers to a “substituted amino” of the formula (—NR^{h}₂), wherein R^{h} is, independently, a hydrogen or an optionally substituted alkyl group as defined herein, and the nitrogen moiety is directly attached to the parent molecule.

The term “aryl” refer to stable aromatic mono- or polycyclic ring system having 3-20 ring atoms, of which all the ring atoms are carbon, and which may be substituted or unsubstituted. In certain embodiments of the present invention, “aryl” refers to a mono, bi, or tricyclic C₄-C₂₀ aromatic ring system having one, two, or three aromatic rings which include, but not limited to, phenyl, biphenyl, naphthyl, and the like, which may bear one or more substituents. Aryl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “arylalkyl” refers to an aryl substituted alkyl group, wherein the terms “aryl” and “alkyl” are defined herein, and wherein the aryl group is attached to the alkyl group, which in turn is attached to the parent molecule. Exemplary arylalkyl groups are benzyl and phenethyl.

The term “aryloxy” refers to a “substituted hydroxyl” of the formula (—OR), wherein R¹ is an optionally substituted aryl group as defined herein, and the oxygen moiety is directly attached to the parent molecule.

The term “arylamino,” refers to a “substituted amino” of the formula (—NR^{h}₂), wherein R^{h} is, independently, a hydrogen or an optionally substituted aryl group as defined herein, and the nitrogen moiety is directly attached to the parent molecule.

The term “arylthioxy” refers to a “substituted thiol” of the formula (—SR), wherein R is an optionally substituted aryl group as defined herein, and the sulfur moiety is directly attached to the parent molecule.

The terms “halo” and “halogen” refer to an atom selected from fluorine (fluoro, —F), chlorine (chloro, —Cl), bromine (bromo, —Br), and iodine (iodo, —I).

The term “heteroaliphatic” refers to an aliphatic moiety, as defined herein, which includes both saturated and unsaturated, nonaromatic, straight chain (i.e., unbranched), branched, acyclic, cyclic (e.g., heterocyclic), or polycyclic hydrocarbons, which are optionally substituted with one or more functional groups, and that contain one or more oxygen, sulfur, nitrogen, phosphorus, or silicon atoms, e.g., in place of carbon atoms. In certain embodiments, heteroaliphatic moieties are substituted by independent replacement of one or more of the hydrogen atoms thereon with one or more substituents. As will be appreciated by one of ordinary skill in the art, “heteroaliphatic” is intended herein to include, but is not limited to, heteroalkyl, heteroalkenyl, heteroalkynyl, heterocycloalkyl, heterocycloalkenyl, and heterocycloalkynyl moieties. Thus, the term “heteroaliphatic” includes the terms “heteroalkyl,” “heteroalkenyl”, “heteroalkynyl”, and the like. Furthermore, the terms “heteroalkyl”, “heteroalkenyl”, “heteroalkynyl”, and the like encompass both substituted and unsubstituted groups. In certain embodiments, “heteroaliphatic” is used to indicate those heteroaliphatic groups (cyclic, acyclic, substituted, unsubstituted, branched or unbranched) having 1-20 carbon atoms. Heteroaliphatic group substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroalkyl” refers to an alkyl moiety, as defined herein, which contain one or more oxygen, sulfur, nitrogen, phosphorus, or silicon atoms, e.g., in place of carbon atoms.

The term “heteroalkenyl” refers to an alkenyl moiety, as defined herein, which contain one or more oxygen, sulfur, nitrogen, phosphorus, or silicon atoms, e.g., in place of carbon atoms.

The term “heteroalkyny” refers to an alkynyl moiety, as defined herein, which contain one or more oxygen, sulfur, nitrogen, phosphorus, or silicon atoms, e.g., in place of carbon atoms.

The term “heteroalkylamino” refers to a “substituted amino” of the formula (—NR^{h}₂), wherein R^{h} is, independently, a hydrogen or an optionally substituted heteroalkyl group, as defined herein, and the nitrogen moiety is directly attached to the parent molecule.

The term “heteroalkyloxy” refers to a “substituted hydroxyl” of the formula (—OR), wherein R¹ is an optionally substituted heteroalkyl group, as defined herein, and the oxygen moiety is directly attached to the parent molecule.

The term “heteroalkylthioxy” refers to a “substituted thiol” of the formula (—SR^{r}), wherein R^{r} is an optionally substituted heteroalkyl group, as defined herein, and the sulfur moiety is directly attached to the parent molecule.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆carbocyclyl”). In some embodiments, a carbocyclyl group has 5 to 6 ring carbon atoms (“C₅₋₆carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆cycloalkyl”). In some embodiments, a cycloalkyl group has 5 to 6 ring carbon atoms (“C₅₋₆cycloalkyl”). In some embodiments, a cycloalkyl group has 5 to 10 ring carbon atoms (“C₅₋₁₀ cycloalkyl”). Examples of C₅₋₆cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

The term “heterocyclic,” “heterocycles,” or “heterocyclyl” refers to a cyclic heteroaliphatic group. A heterocyclic group refers to a non-aromatic, partially unsaturated or fully saturated, 3- to 12-membered ring system, which includes single rings of 3 to 8 atoms in size, and bi- and tri-cyclic ring systems which may include aromatic five- or six-membered aryl or heteroaryl groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocyclic refers to a non-aromatic 5-, 6-, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Heterocyclyl groups include, but are not limited to, a bi- or tri-cyclic group, comprising fused five, six, or seven-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Exemplary heterocycles include azacyclopropanyl, azacyclobutanyl, 1,3-diazatidinyl, piperidinyl, piperazinyl, azocanyl, thiaranyl, thietanyl, tetrahydrothiophenyl, dithiolanyl, thiacyclohexanyl, oxiranyl, oxetanyl, tetrahydrofuranyl, tetrahydropuranyl, dioxanyl, oxathiolanyl, morpholinyl, thioxanyl, tetrahydronaphthyl, and the like, which may bear one or more substituents. Substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroaryl” refer to stable aromatic mono- or polycyclic ring system having 3-20 ring atoms, of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms. Exemplary heteroaryls include, but are not limited to pyrrolyl, pyrazolyl, imidazolyl, pyridinyl (pyridyl), pyrimidinyl, pyrazinyl, pyridazinyl, triazinyl, tetrazinyl, pyyrolizinyl, indolyl, quinolinyl, isoquinolinyl, benzoimidazolyl, indazolyl, quinolinyl, isoquinolinyl, quinolizinyl, cinnolinyl, quinazolynyl, phthalazinyl, naphthridinyl, quinoxalinyl, thiophenyl, thianaphthenyl, furanyl, benzofuranyl, benzothiazolyl, thiazolynyl, isothiazolyl, thiadiazolynyl, oxazolyl, isoxazolyl, oxadiaziolyl, oxadiaziolyl, and the like, which may bear one or more substituents. Heteroaryl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, sulfinyl, sulfonyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “heteroarylamino” refers to a “substituted amino” of the (—NR^{h}₂), wherein R^{h} is, independently, hydrogen or an optionally substituted heteroaryl group, as defined herein, and the nitrogen moiety is directly attached to the parent molecule.

The term “heteroaryloxy” refers to a “substituted hydroxyl” of the formula (—OR¹), wherein R¹ is an optionally substituted heteroaryl group, as defined herein, and the oxygen moiety is directly attached to the parent molecule.

The term “heteroarylthioxy” refers to a “substituted thiol” of the formula (—SR), wherein R^{r} is an optionally substituted heteroaryl group, as defined herein, and the sulfur moiety is directly attached to the parent molecule.

The term “hydroxyl” or “hydroxyl” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “imino” refers to a group of the formula (═NR), wherein R corresponds to hydrogen or any substituent as described herein, that results in the formation of a stable moiety (for example, a suitable amino protecting group; aliphatic, alkyl, alkenyl, alkynyl, heteroaliphatic, heterocyclic, aryl, heteroaryl, acyl, amino, hydroxyl, alkylaryl, arylalkyl, and the like, each of which may or may not be further substituted). In certain embodiments, imino refers to ═NH wherein R^{r} is hydrogen.

The term “nitro” refers to a group of the formula (—NO₂).

The term “oxo” refers to a group of the formula (═O).

A “protecting group” is well known in the art and include those described in detail in Greene's Protective Groups in Organic Synthesis, P. G. M. Wuts and T. W. Greene, 4^{th} edition, Wiley-Interscience, 2006, the entirety of which is incorporated herein by reference.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R⁰)₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl, (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), .beta.-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl] amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein.

Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, .alpha.-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxy acetate, 2,6-dichloro-4-(1,1,3,3-tetramethylbutyl)phenoxy acetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F, Cl, Br, I), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

The term “polymorphs” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The following abbreviations are used throughout: N-acetyl galactosamine (GalNAc); Thin-layer chromatography (TLC); Liquid chromatography-mass spectrometry (LC-MS); High Performance Liquid Chromatography (HPLC); dichloroethane (DCE); dichloromethane (DCM); Trimethylsilyl trifluoromethanesulfonate (TMSOTf); N,N′-diisopropylcarbodiimide (DIC); dimethylaminopyridine (DMAP); ethylacetate (EA); dimethyl sulfoxide (DMSO); trifluoroacetic acid (TFA); acetonitrile (ACN); 2-(1H-benzotriazole-1-yl)-1,1,3,3-tetramethylaminium tetrafluoroborate (TBTU); tetrahydrofuran (THF); dimethoxytrityl (DMT); controlled pore glass (CPG); 5-ethylthio-1H-tetrazole (ETT); phenylacetyl disulfide (PADS); trimethylamine (TEA); hexafluoroisopropanol (HFIP); hexylamine (HA); phosphate-buffered saline (PBS); and ion-pair reversed-phase (IP-RP).

### Targeted Ligand Clusters

### Formula 1

In at least some embodiments of the invention a targeting ligand cluster has the general structure of Formula 1:

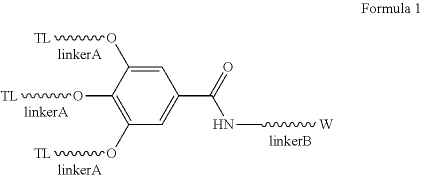

where: TL is one or more targeting ligands, including but not limited to: N-acetylgalactosamine, galactose, galactosamine, N-formyl-galactosamine, N-propionylgalactosamine, N-n-butanoylgalactosamine, and N-iso-butanoylgalactosamine; one or more TLs may be different from one or more other TLs of the same targeting ligand cluster;

linkerA is one or more bifunctional spacers, with one end of linkerA attaching to the targeting ligand and the other end attaching to a phenolic hydroxy group of gallic acid through an ether bond;

linkerB is a bifunctional spacer, with one end of linkerB attaching to a phosphoramidite or an oligonucleotide and the other end attaching to the carboxylic acid of gallic acid through an amide bond; and

W is H, a protecting group, phosphoramidite or oligonucleotide.

### Formula 2

In at least some embodiments, a targeting ligand cluster of the invention comprises the following general structure of Formula 2:

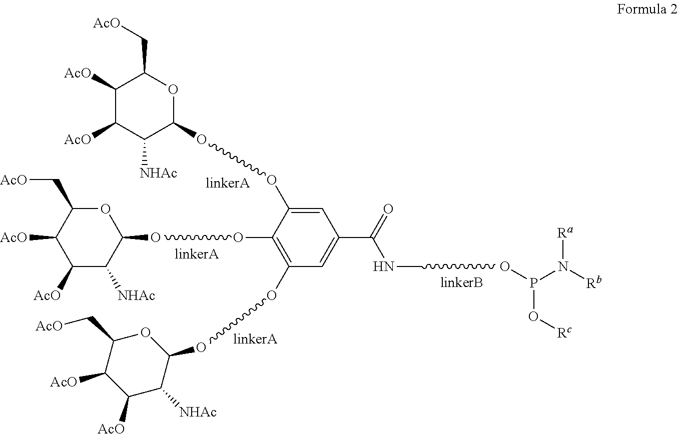

where: linkerA is at least one spacer, with one end of linkerA attaching to a GalNAc targeting ligand and the other end attaching to a phenolic hydroxy group of gallic acid through an ether bond; in at least some embodiments, linkerA may include at least one of polyethylene glycol (PEG), an alkyl group, a cycloalkyl group, an alkenyl group, a cycloalkenyl group, an alkynyl group, an aryl group, an aralkyl group, an aralkenyl group, and an aralkynyl group; in at least some embodiments, linkerA includes one or more heteroatoms, aliphatic heterocycles, heteroaryls, amino acids, nucleotides, and saccharides;

linkerB is at least one spacer, with one end of linkerB attaching to a phosphorous atom of a phosphoramidite or an oligonucleotide and the other end attaching to the carboxylic acid of gallic acid through an amide bond; in at least some embodiments, linkerB may include at least one of PEG, an alkyl group, a cycloalkyl group, an alkenyl group, a cycloalkenyl group, an alkynyl group, an aryl group, an aralkyl group, an aralkenyl group, and an aralkynyl group; in at least some embodiments, linkerB includes one or more heteroatoms, aliphatic heterocycles, heteroaryls, amino acids, nucleotides, and saccharides;

R^{a} may be a C₁ to C₆ alkyl, C₃ to C₆ cycloalkyl, or R^{a} may join with R^{b} through a nitrogen atom to form a cycle; in at least some embodiments, R^{a} may be an isopropyl group;

R^{b} may be a C₁ to C₆ alkyl, C₃ to C₆ cycloalkyl, or R^{b} may join with R^{a} through a nitrogen atom to form a cycle; in at least some embodiments, R^{b} may be an isopropyl group; and in at least some embodiments, R^{C} may be a phosphite and phosphate protecting group; in at least some embodiments, the phosphate protecting group may include at least one of methyl, allyl, 2-cyanoethyl, 4-cyano-2-butenyl, 2-cyano-1,1-dimethylethyl, 2-(trimethylsilyl)ethyl, 2-(S-acetylthio)ethyl, 2-(S-pivaloylthio)ethyl, 2-(4-nitrophenyl)ethyl, 2,2,2-trichloroethyl, 2,2,2-trichloro-1,1-dimethylethyl, 1,1,1,3,3,3-hexafluoro-2-propyl, fluorenyl-9-methyl, 2-chlorophenyl, 4-chlorophenyl, and 2,4-dichlorophenyl; in at least some embodiments, R^{C} may be a 2-cyanoethyl group.

In at least some embodiments, linkerA may include one or more of:

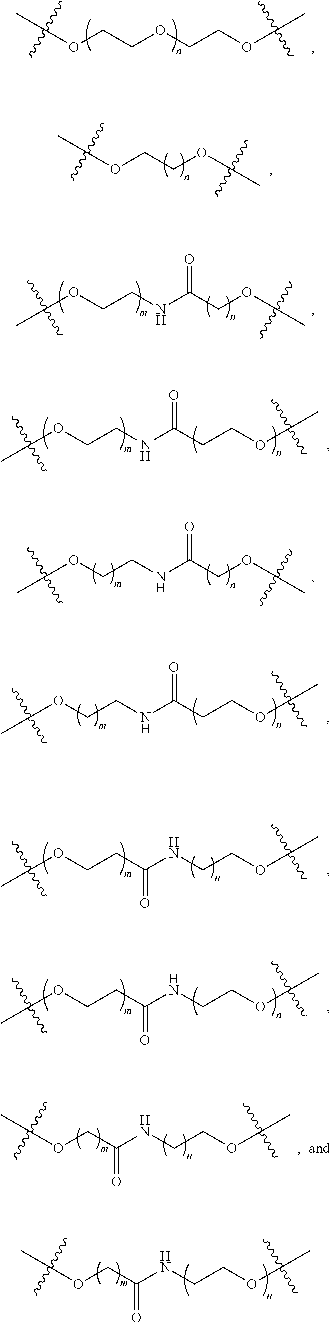

where:

m may be an integral number 1, 2, 3, 4, 5, 6, 7, 8, 9, 10, 11, or 12; and

n may be an integral number 1, 2, 3, 4, 5, 6, 7, 8, 9, 10, 11, or 12.

In at least some embodiments, linkerB may include one or more of:

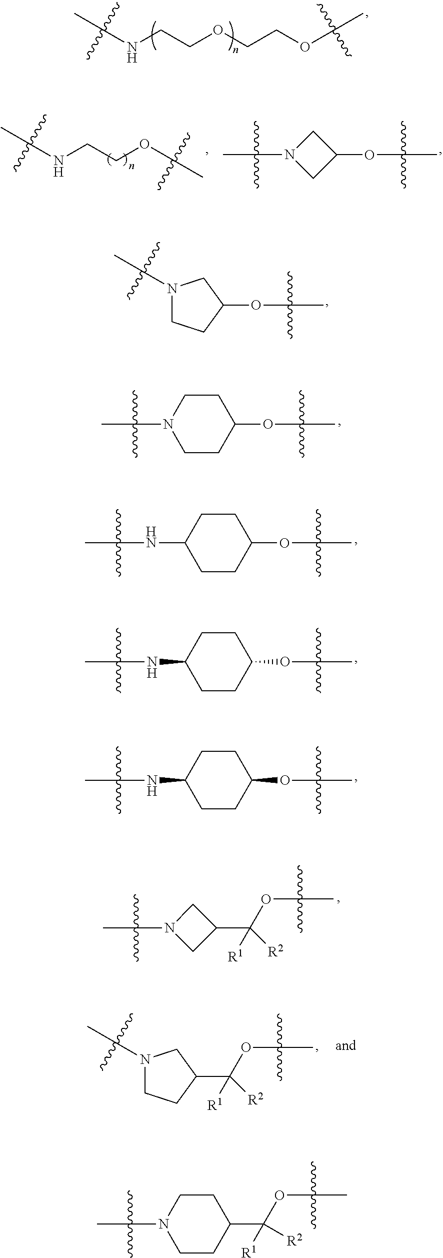

where: n may be an integral number 1, 2, 3, 4, 5, 6, 7, 8, 9, 10, 11, or 12;

R¹ may be H, methyl (Me), ethyl (Et), cyclopropyl, or R¹ may join with R² through a carbon atom to form a 3-6 member ring; and

R² may be H, Me, Et, cyclopropyl, or R² may join with R¹ through a carbon atom to form a 3-6 member ring.

In at least some embodiments, linkerB may include one or more of:

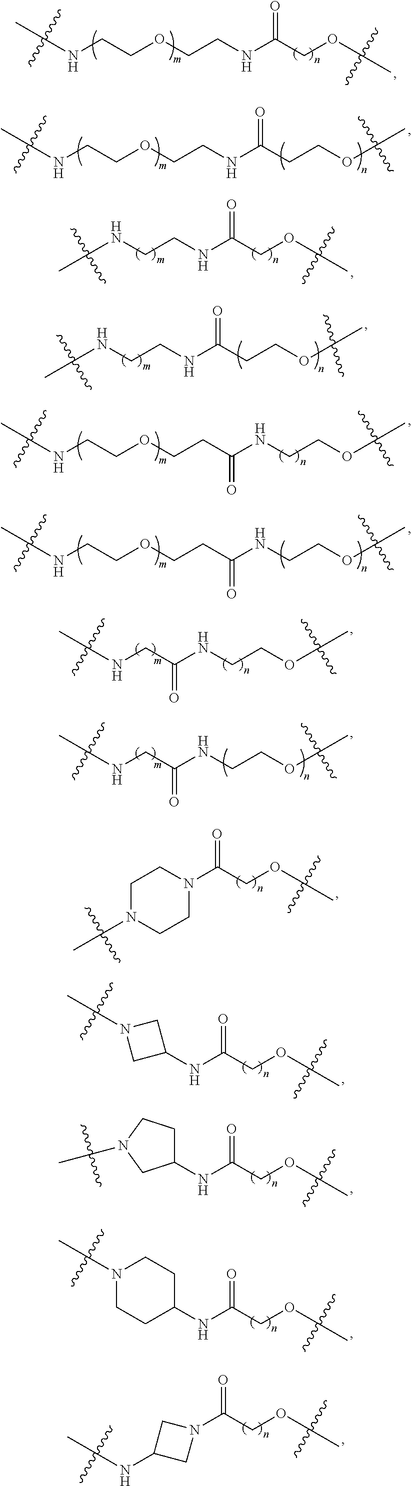

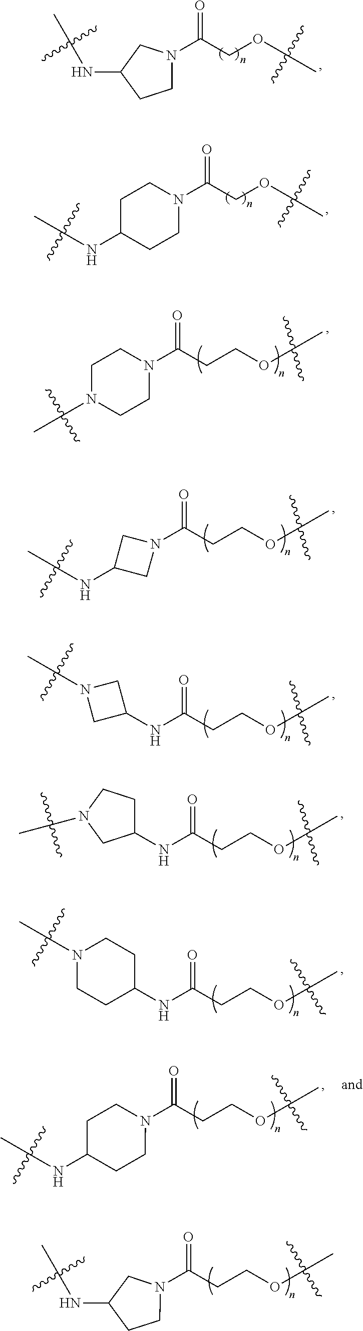

where: m may be an integral number 1, 2, 3, 4, 5, 6, 7, 8, 9, 10, 11, or 12; and

n may be an integral number 1, 2, 3, 4, 5, 6, 7, 8, 9, 10, 11, or 12.

### Formula 3

In at least some embodiments of compounds of the invention comprising a targeting ligand cluster comprises the following general structure of Formula 3:

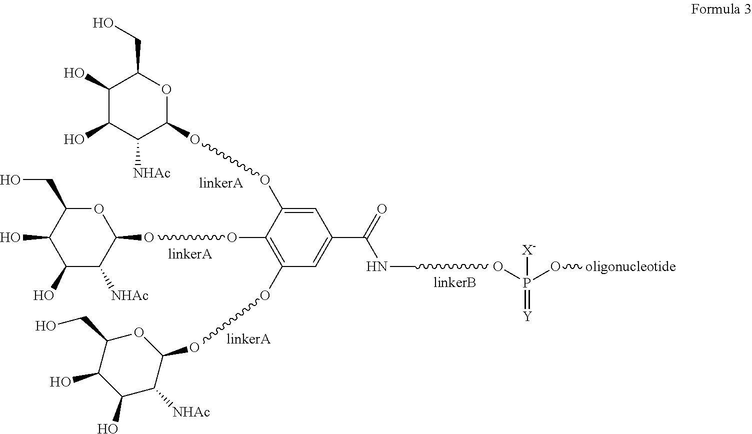

where: oligonucleotide includes at least one of a small interfering RNA (siRNA), a single strand siRNA, a microRNA (miRNA), an antisense oligonucleotide, a messenger RNA (mRNA), a ribozyme, a plasmid, an immune stimulating nucleic acid, an antagomir, and an aptamer;

X is at least one of oxygen (O) and sulfur (S);

Y is at least one of O, S, and NH;

linkerA is at least one spacer, with one end of linkerA attaching to a GalNAc targeting ligand and the other end attaching to a phenolic hydroxy group of gallic acid through an ether bond; in at least some embodiments, linkerA may include at least one of PEG, an alkyl group, a cycloalkyl group, an alkenyl group, a cycloalkenyl group, an alkynyl group, an aryl group, an aralkyl group, an aralkenyl group, and an aralkynyl group; in at least some embodiments, linkerA includes one or more heteroatoms, aliphatic heterocycles, heteroaryls, amino acids, nucleotides, and saccharides; and

linkerB is at least one spacer, with one end of linkerB attaching to a phosphorous atom of a phosphoramidite or an oligonucleotide and the other end attaching to the carboxylic acid of gallic acid through an amide bond; in at least some embodiments, linkerB may include at least one of PEG, an alkyl group, a cycloalkyl group, an alkenyl group, a cycloalkenyl group, an alkynyl group, an aryl group, an aralkyl group, an aralkenyl group, and an aralkynyl group; in at least some embodiments, linkerB includes one or more heteroatoms, aliphatic heterocycles, heteroaryls, amino acids, nucleotides, and saccharides.

In at least some embodiments, linkerA may include one or more of:

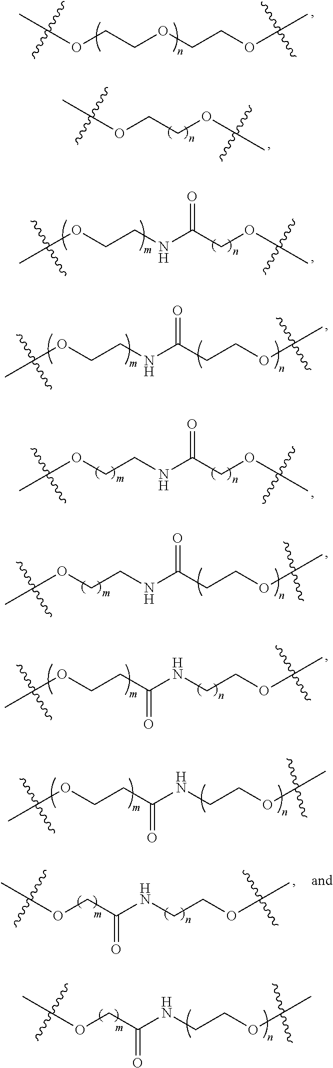

where:

m may be an integral number 1, 2, 3, 4, 5, 6, 7, 8, 9, 11, or 12; and

n may be an integral number 1, 2, 3, 4, 5, 6, 7, 8, 9, 10, 11, or 12.

In at least some embodiments, linkerB may include one or more of:

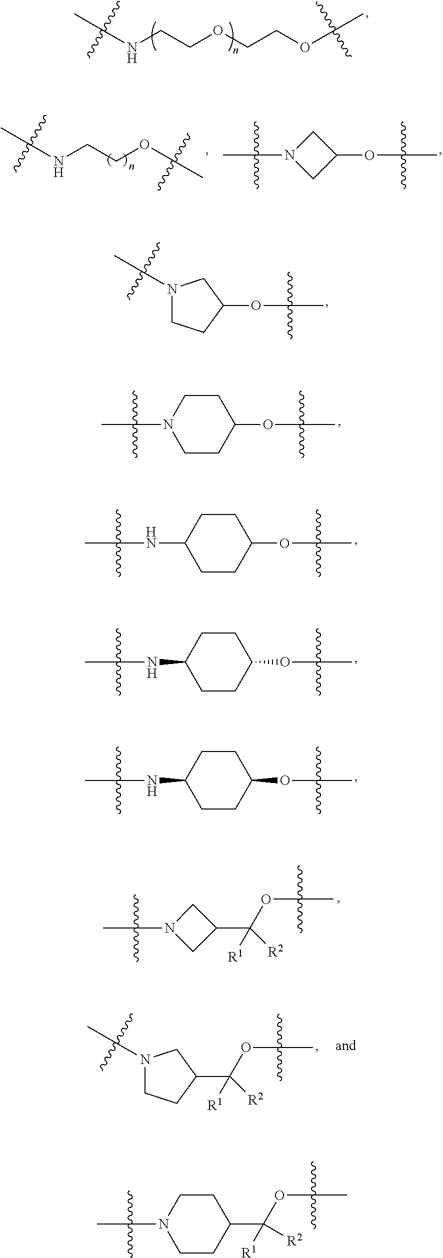

where: n may be an integral number 1, 2, 3, 4, 5, 6, 7, 8, 9, 10, 11, or 12;

R¹ may be H, Me, Et, cyclopropyl, or R may join with R² through a carbon atom to form a 3-6 member ring; and

R² may be H, Me, Et, cyclopropyl, or R² may join with R¹ through a carbon atom to form a 3-6 member ring.

In at least some embodiments, linkerB may include one or more of:

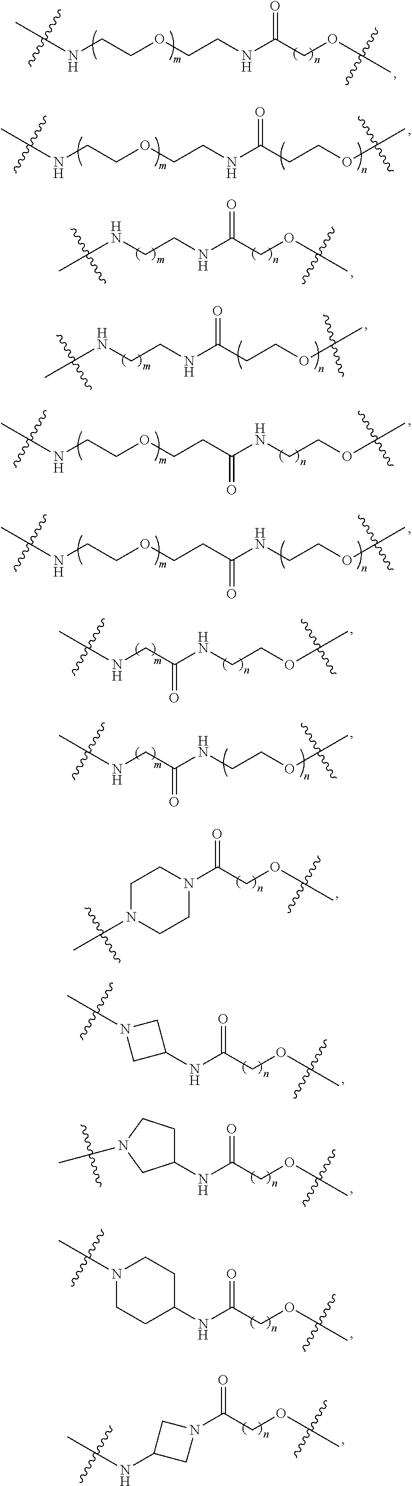

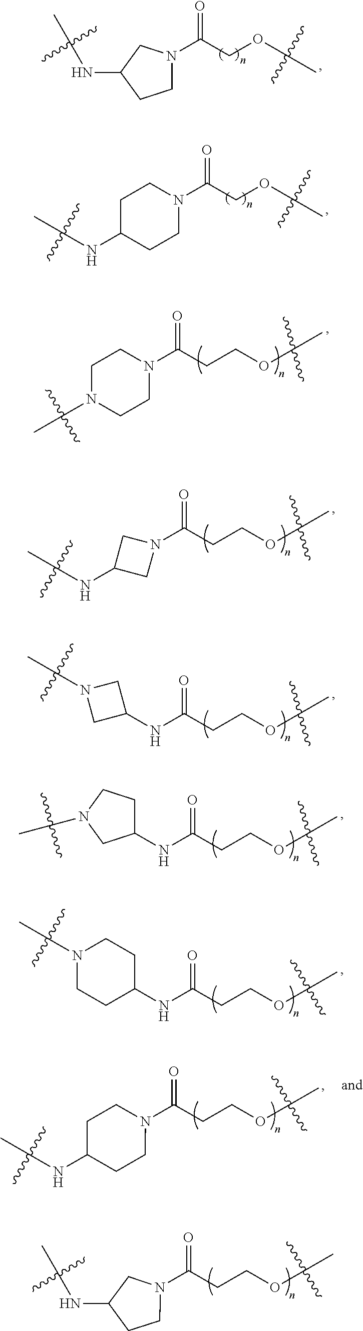

Wherein m may be an integral number 1, 1, 2, 3, 4, 5, 6, 7, 8, 9, 10, 11, or 12; and n may be an integral number 1, 2, 3, 4, 5, 6, 7, 8, 9, 10, 11, or 12.

It will be understood that each LinkerA included in a targeting ligand cluster of the invention may be independently selected, meaning (1) the LinkerAs in the targeting ligand cluster are all the same as each other, (2) two of the LinkerAs in the targeting ligand cluster are the same as each other and one is different from the two; or (3) each of the three LinkerAs in the targeting ligand cluster is different from the others. It will be understood that in a targeting ligand cluster of the invention comprising more than one LinkerB, each LinkerB is independently selected, meaning (4) all the LinkerBs in a targeting ligand cluster are the same as each other, (5) two or more of the LinkerBs in a targeting ligand cluster are the same as each other and at least one LinkerB is different from the two or more, or (6) each LinkerB in a targeting ligand cluster is different from all of the other LinkerBs in the targeting ligand cluster. The terms “first linker” and “linkerA” may be used herein interchangeably. The terms “second linker” and “linkerB” may be used herein interchangeably. As used herein the terms “targeting ligand cluster” and “ligand cluster” may be used interchangeably.

It has now been demonstrated that embodiments of GalNAc phosphoramidite targeting ligand clusters of the invention can be used with standard oligonucleotide synthesis and deprotection methods. Oligonucleotides containing a GalNAc targeting ligand cluster can be deprotected using standard procedures with which the acetyl protecting groups on the GalNAc group are removed. Certain embodiments of methods of the invention include conjugating an oligonucleotide to a GalNAc targeting ligand cluster of the invention. In some embodiments of methods of the invention a protected GalNAc targeting ligand phosphoramidite is used in a conjugation method and such methods can be used for efficient conjugation resulting in high yields and high purity levels of the conjugated product. Various examples herein include GalNAC phosphoramidite targeting ligand clusters. In some embodiments of the invention a targeting ligand cluster may include a phosphoramidite as set forth in Ligands A-WW shown herein. Ligand A, Ligand B, Ligand C, Ligand D, Ligand E, Ligand F, Ligand G, Ligand H, Ligand I, Ligand J, Ligand K, Ligand L, Ligand M, Ligand N, Ligand O, Ligand P, Ligand Q, Ligand R, Ligand S, Ligand T, Ligand U, Ligand V, Ligand W, Ligand X, Ligand Y, Ligand Z, Ligand JJ, Ligand KK, Ligand LL, Ligand MM, Ligand NN, Ligand OO, Ligand PP, Ligand QQ, Ligand PR, Ligand SS, Ligand TT, Ligand UU, Ligand VV, and Ligand WW are set forth herein. These 40 Ligands may be referred to herein as Ligands A-WW, or a subset may be referenced by indicating a range of Ligand numbers and/or indicating one or more individual Ligand numbers.

As described elsewhere herein, a targeting ligand cluster of Formula 1 or Formula 2 can be attached to an oligonucleotide compound. When describing attachment of a targeting ligand cluster of the invention and an oligonucleotide, the terms “attached”, “attachment”, and “attach” may be used interchangeably herein with the terms: “conjugated”, “conjugation”, and “conjugate”; and “joined”, “joining”, and “join”, respectively.

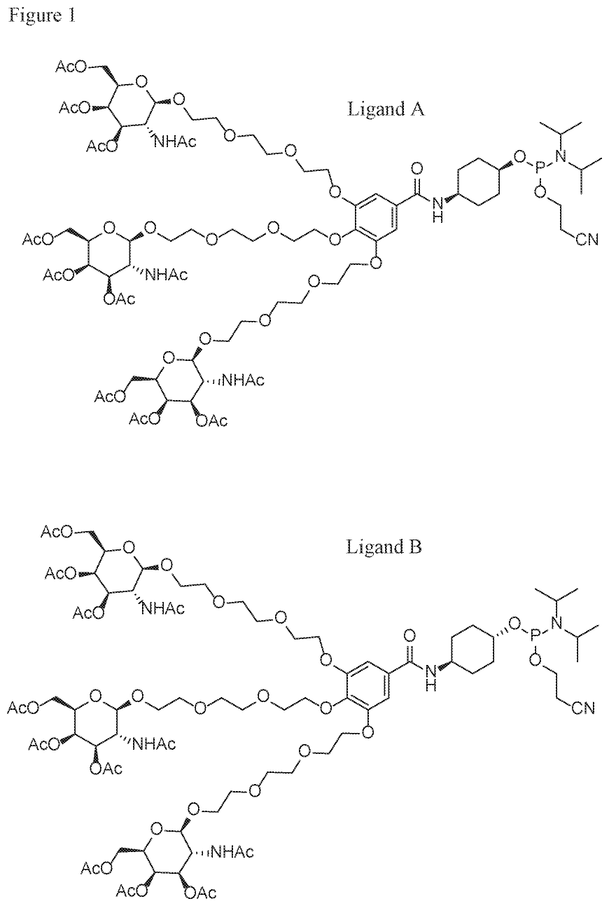

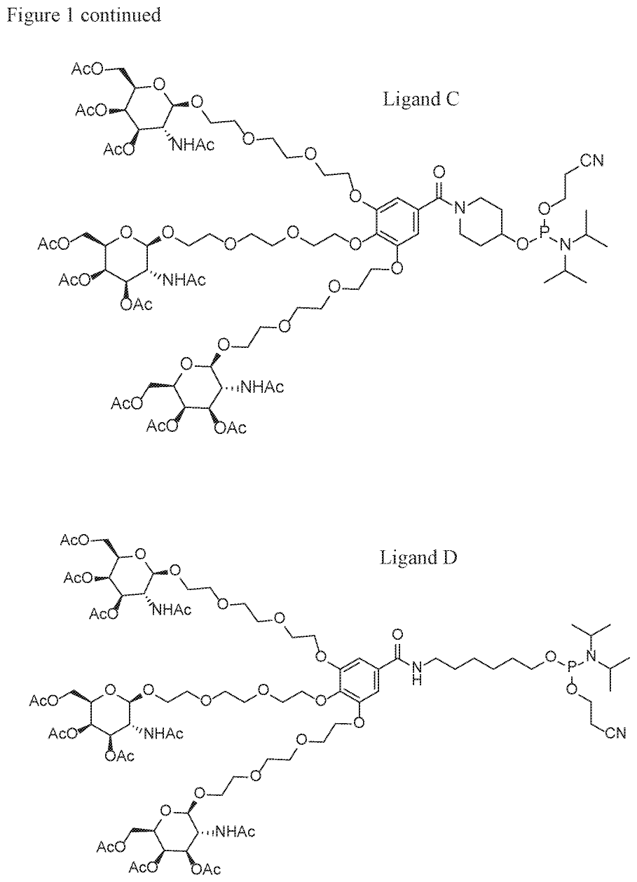

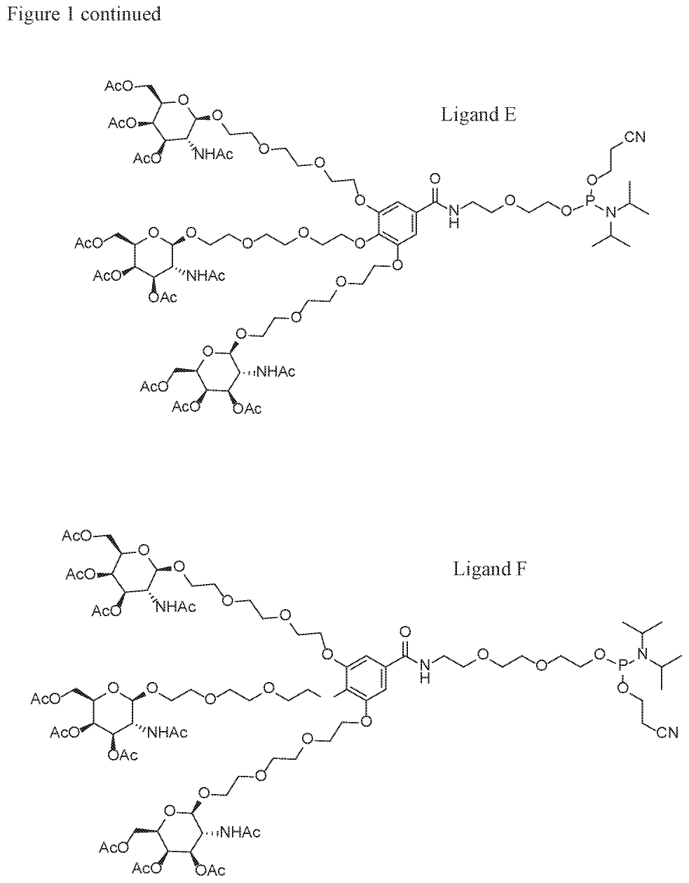

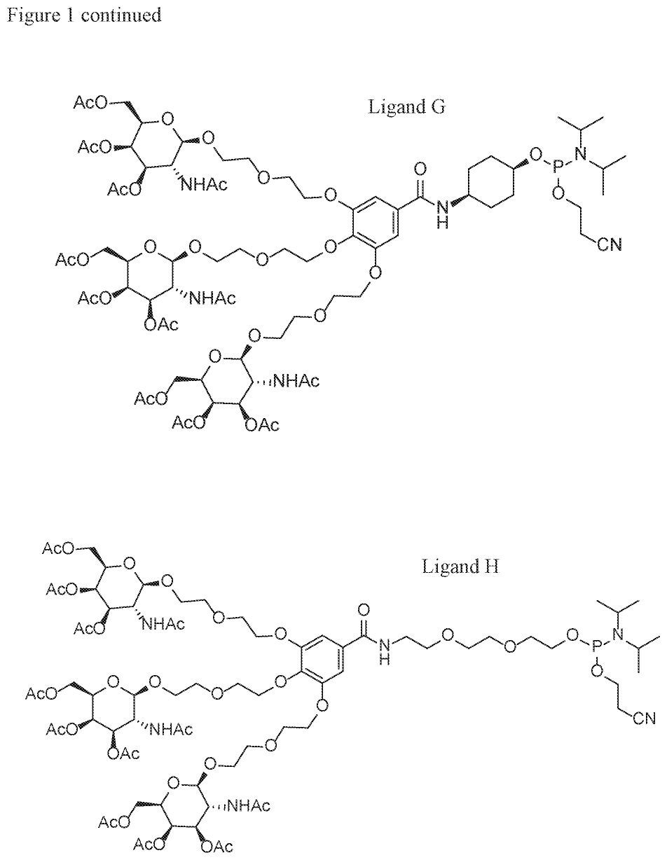

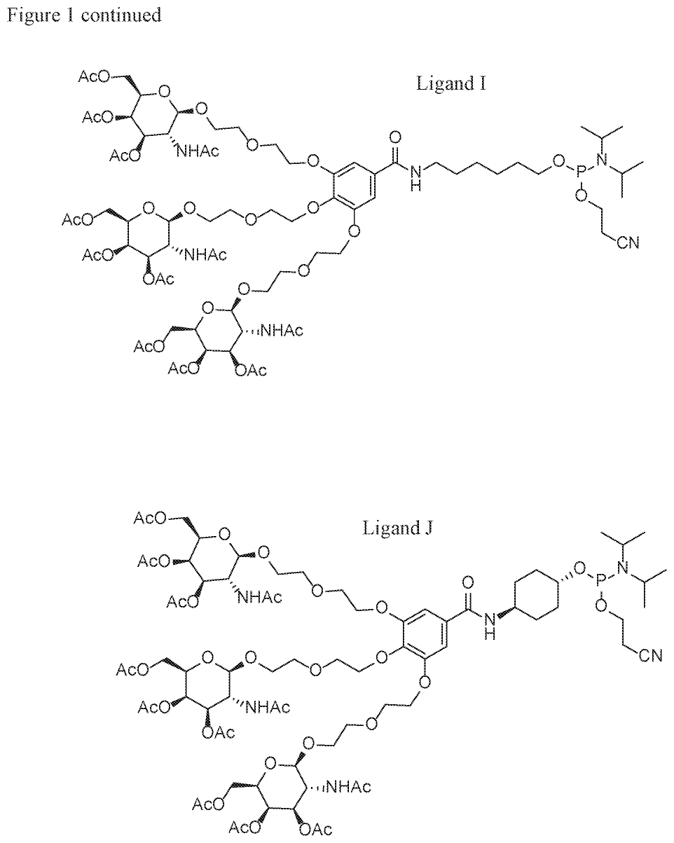

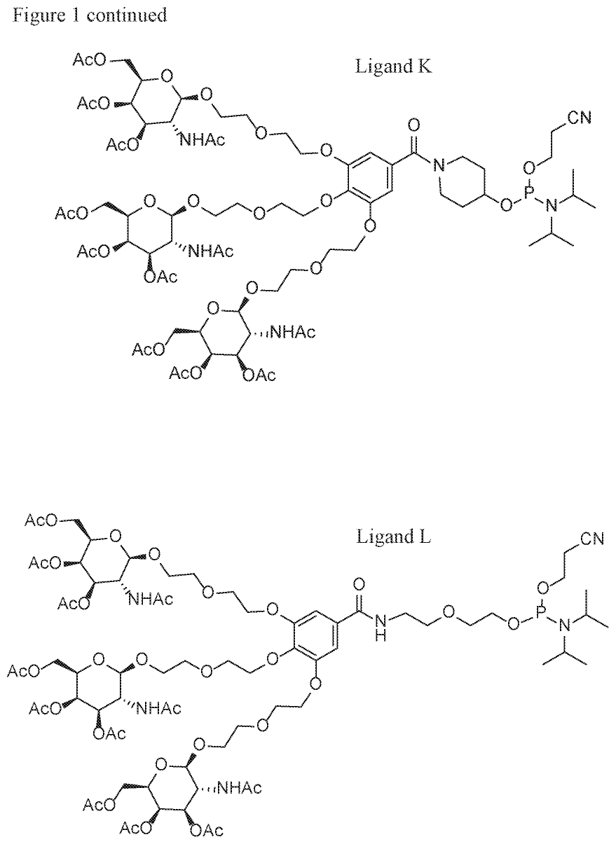

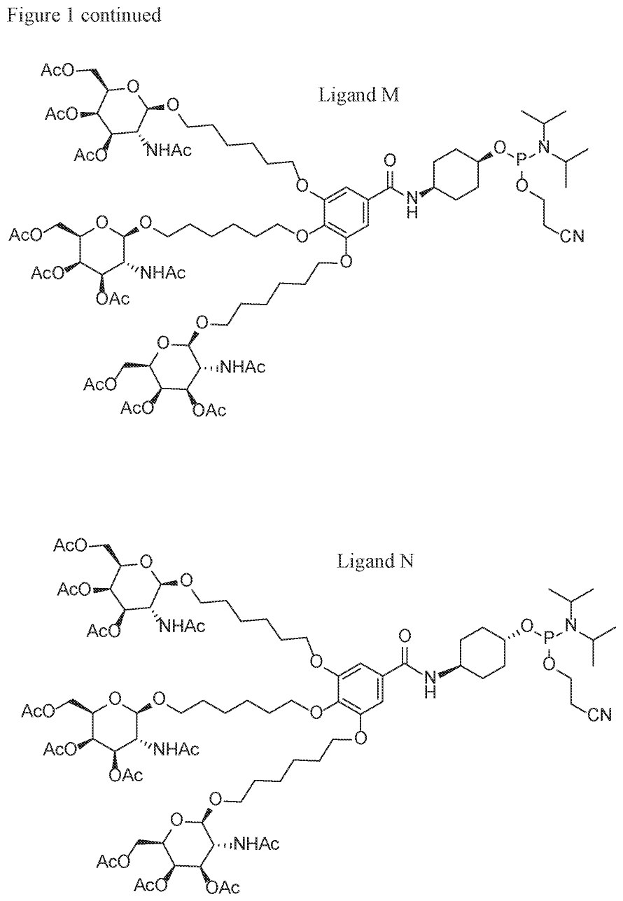

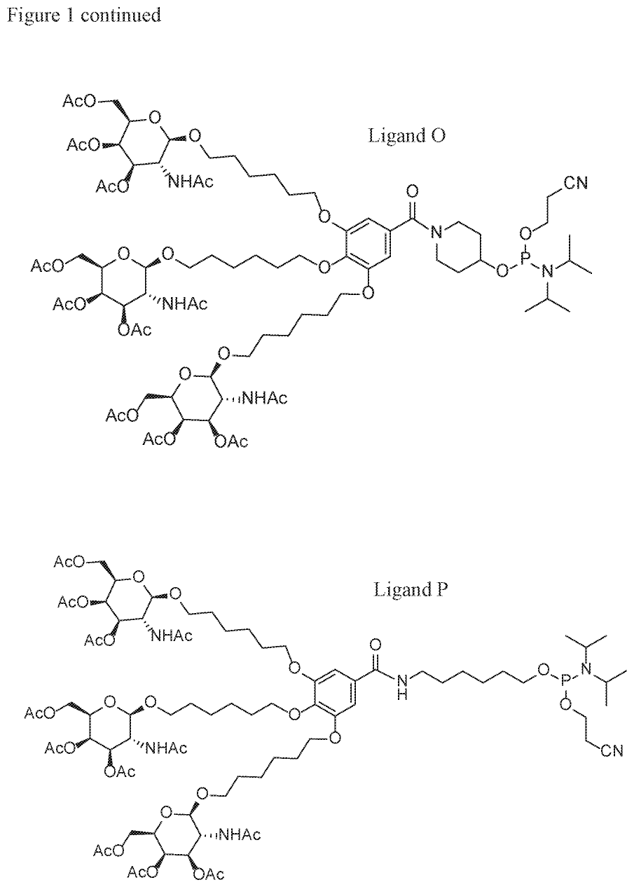

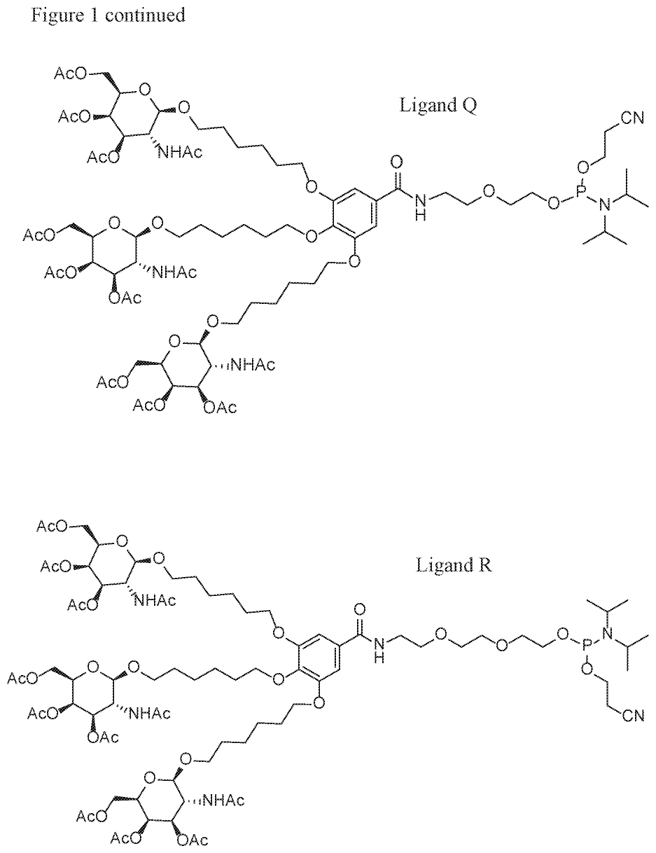

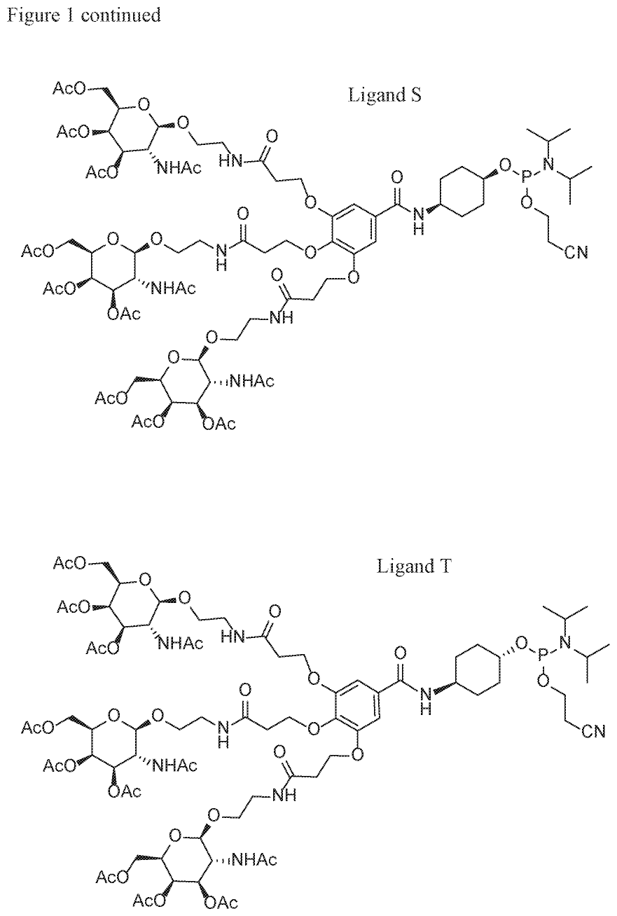

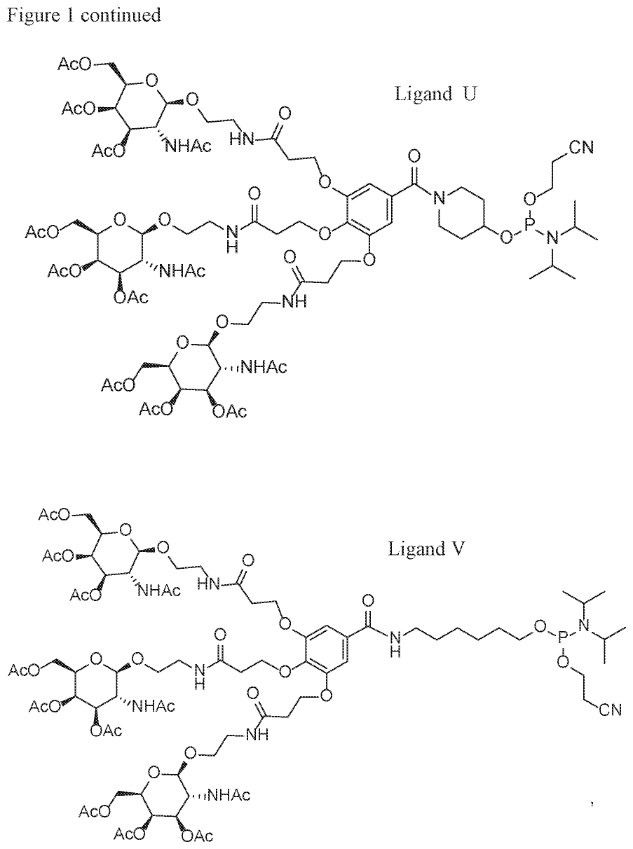

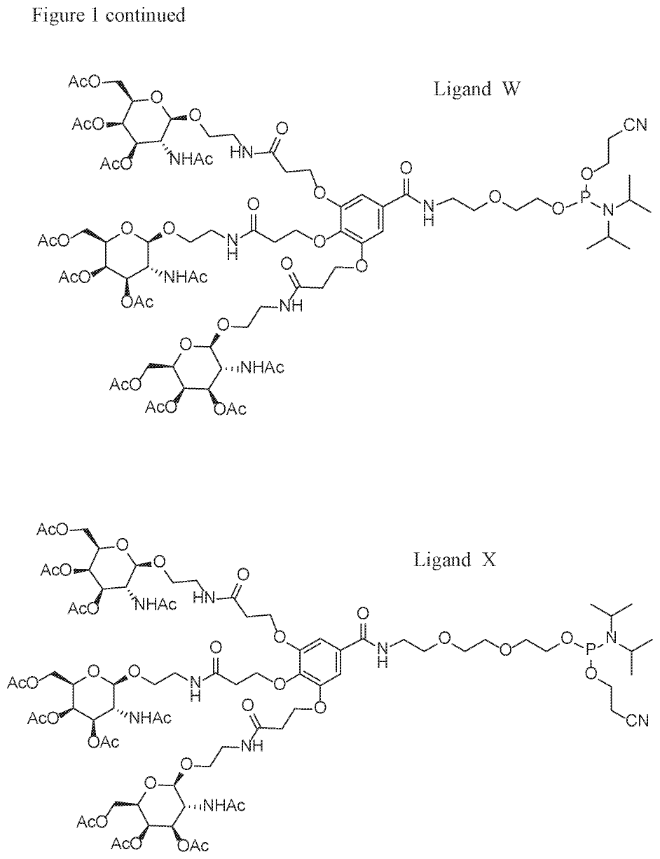

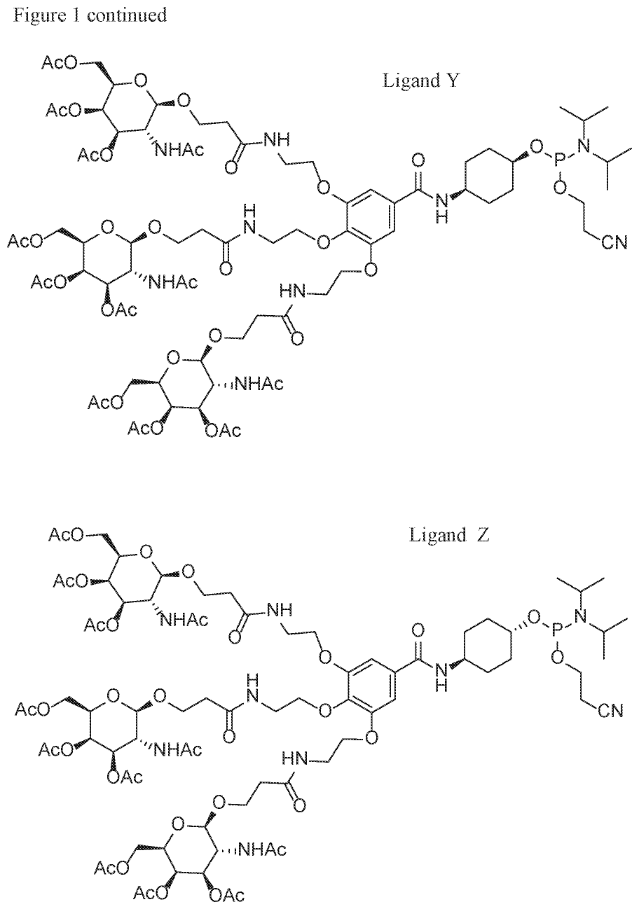

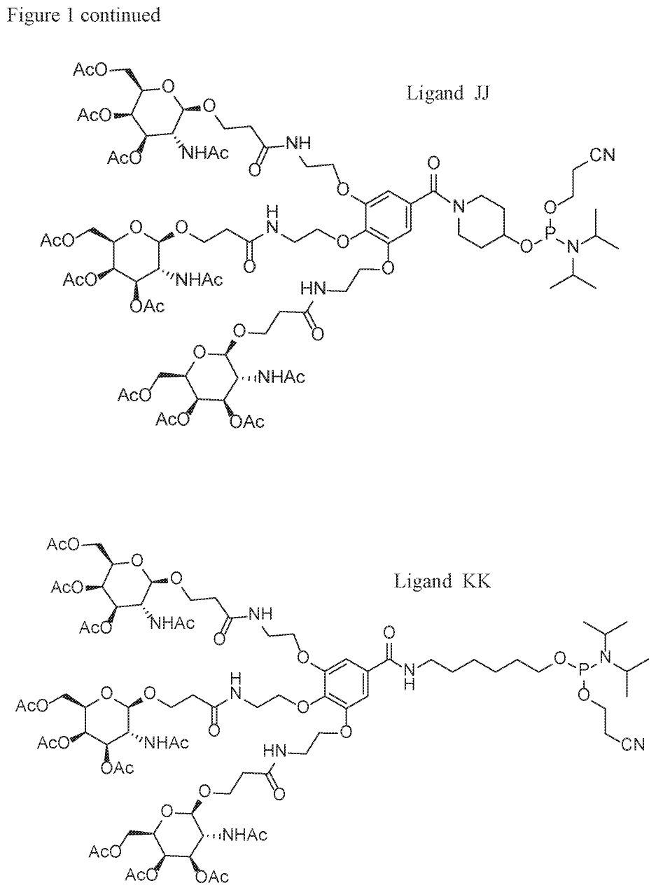

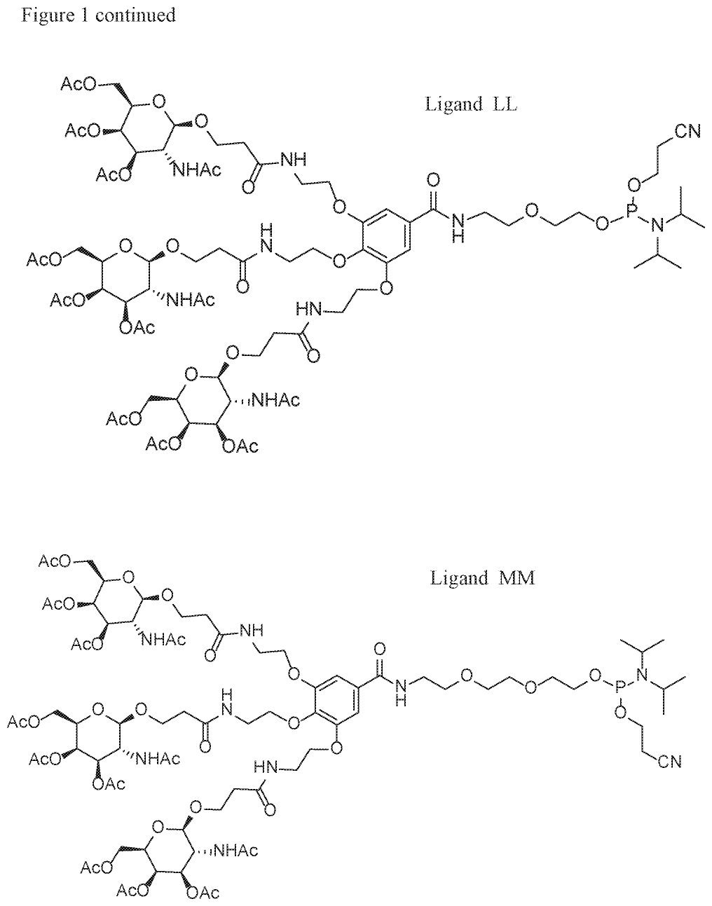

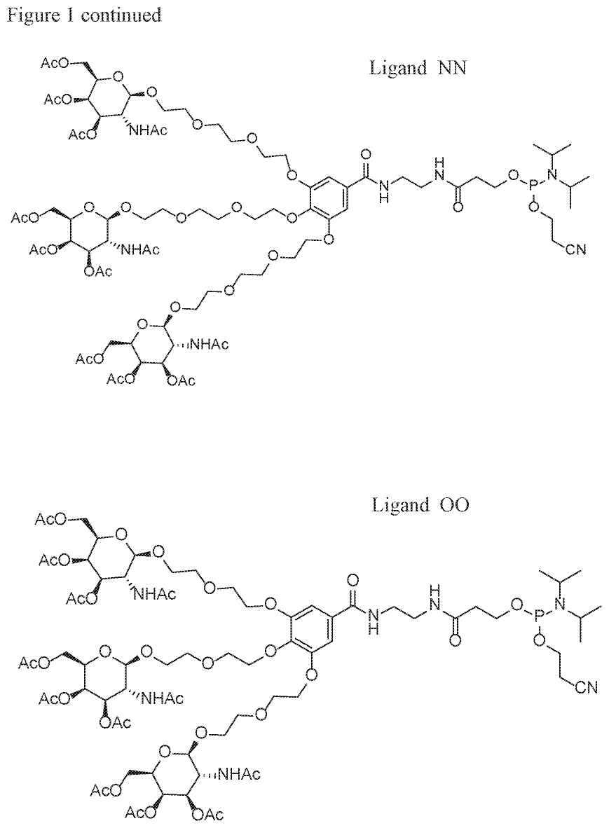

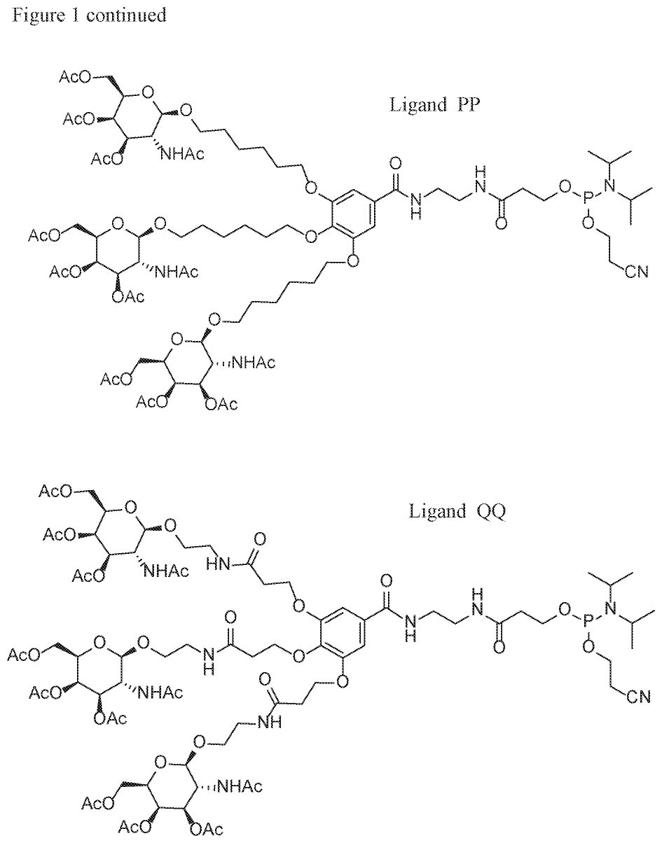

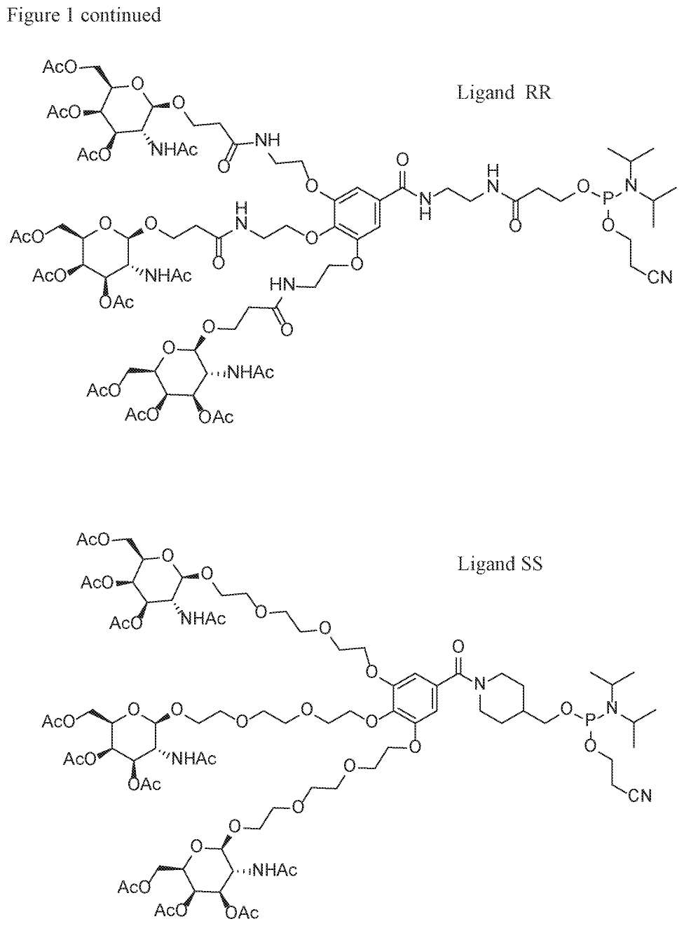

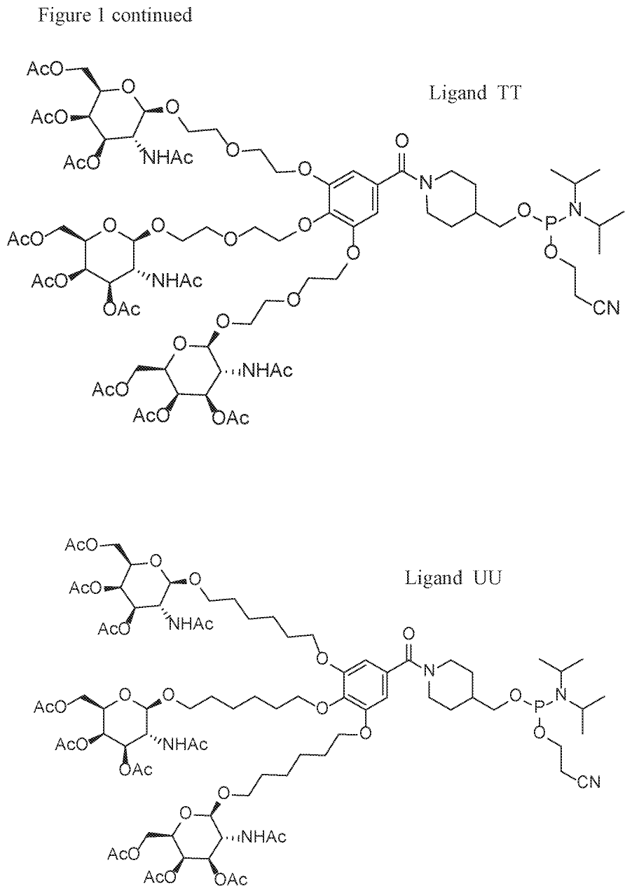

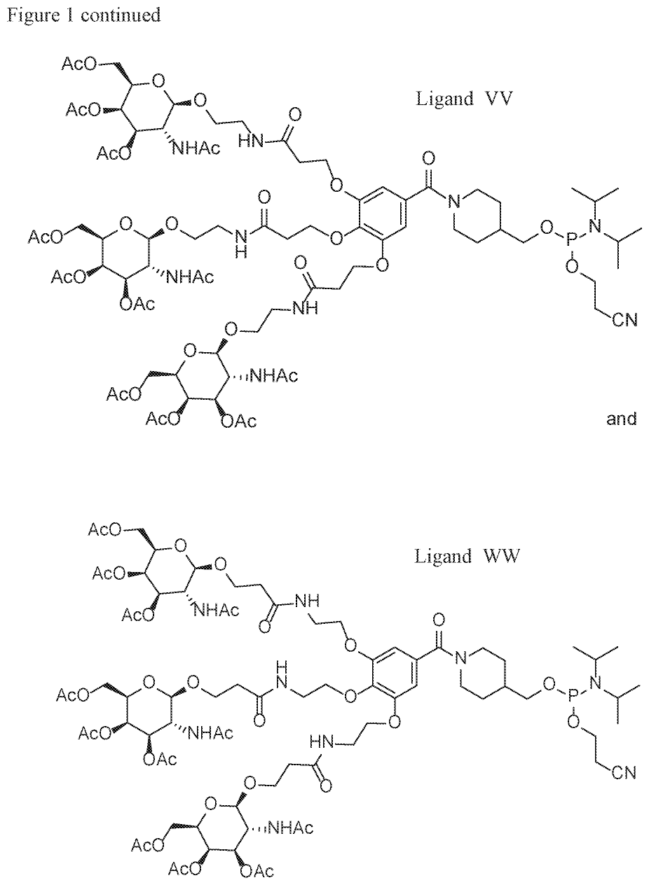

Some embodiments of a targeting ligand cluster of the invention are shown herein as Ligands A-WW (FIG. 1). In certain embodiments of compositions and/or method of the invention, a targeting ligand cluster comprises one of Ligands A-WW, (which may also be referred to herein as “Compounds A-WW”), which is attached to a nucleic acid molecule and/or a compound comprising a nucleic acid. In some embodiments, a targeting ligand cluster of the invention is attached to at least one nucleic acid molecule, and the resulting complex may be referred to herein as a “targeting ligand cluster/nucleic acid complex. In some embodiments of the invention, a nucleic acid molecule included in a targeting ligand cluster/nucleic acid complex comprises an oligonucleotide. A general formula of a targeting ligand cluster/nucleic acid complex of the invention is shown herein as Formula 3, which shows a targeting ligand cluster attached to an oligonucleotide. Non-limiting examples of ligand cluster/nucleic acid complexes of the invention include: MITO-A, MITO-B, MITO-C, MITO-D, MITO-E, MITO-F, MITO-G, MITO-H, and MITO-I.

In some embodiments of a targeting ligand cluster/nucleic acid complex of the invention includes an siRNA comprising a FXII siRNA. It will be understood that a targeting ligand cluster of the invention may be conjugated to siRNA molecules other than FXII siRNA. An siRNA molecule may be selected for conjugation with a targeting ligand cluster of the invention on the basis of the gene that is targeted by the siRNA. Thus, if it is of interest to reduce expression of, for example, “protein A” in a cell and/or subject, an siRNA can be selected, attached to a targeting ligand cluster of the invention, and administered to the cell and/or subject. The selection of the siRNA may be at least in part because the selected siRNA is capable of reducing expression of the protein A gene, which may be referred to as the selected siRNA's “target gene.” Embodiments of targeting ligand cluster/nucleic acid complexes of the invention can be administered to a cell and/or subject and deliver a functional siRNA into the cell and/or subject wherein the resulting presence of the siRNA reduces expression of the siRNA's target gene.

### Compounds Comprising One or More PEG Linkers

In at least some embodiments of the invention, polyethylene glycol (PEG) may be used as “linkerA” and/or “linkerB” in Formula 1 herein. The linkerA may be individually selected such that a single compound of Formula 1 may have a single PEG linkerA, two different PEG linkerAs, or three different PEG linkerAs. Moreover, PEGs of various molecular weights may be used, and one PEG linkerA may have a same (or similar) or a different molecular weight than a second PEG linkerA of the same compound.

As illustrated in various example compounds above (and using Formula 1 as a reference), PEG may couple a TL to Gallic acid by the PEG directly bonding to the oxygen of a hydroxyl group of Gallic acid. That is, in at least some embodiments of the invention, only an oxygen may be positioned between PEG and the aromatic functionality of Gallic acid. In a specific non-limiting example, in at least some embodiments of the invention, a nitrogen atom (or nitrogen-containing functionality) may not be positioned between PEG and the aromatic functionality of Gallic acid.

### Synthesis

Preparation, also referred to herein as synthesis, of a compound according to the present closure may include various steps. In at least some embodiments of the invention, the preparation starts with an esterification reaction. In at least some embodiments of the invention, the esterification reaction is followed by a nucleophilic substitution (SN2) reaction, then followed by a glycosylation reaction, then followed by a deprotection reaction (e.g., of t-butylester). In at least some embodiments of the invention, the deprotection reaction is followed by an amide coupling reaction. In at least some embodiments of the invention, the amide coupling reaction is followed by a phosphorylation reaction. One skilled in the art will appreciate that the foregoing illustrating preparation method may be altered depending on which starting and intermediate materials are used.

### Synthesis Scheme 1

An embodiment of a method for preparing a compound of the invention based on general Formula 2 (shown below and in Example 1) is identified as “Synthesis Scheme 1.” Further details of Synthesis Scheme 1 and additional synthetic methods that may be used to prepare and use an embodiment of a targeting ligand cluster using gallic acid as a scaffold are provided in Example 1. The Examples herein also set forth synthetic methods to prepare certain embodiments of targeting ligand clusters of the invention, for example, embodiments of synthesis methods for preparing Ligands of the invention, including Ligands A-WW, are shown in the Examples section herein.

Synthesis Scheme 1 illustrates synthetic means to prepare targeting ligand clusters having general Formula 1. Compounds and intermediaries shown in Synthesis Scheme 1 are identified with assigned Roman numerals (i)-(vii). It will be understood that the compounds and/or intermediaries may be identified elsewhere herein with Arabic numbers instead of the Roman numerals and descriptions of characteristics of compounds having Roman numerals (i)-(vii) also apply to the corresponding Arabic numbered compounds and/or intermediaries, respectively.

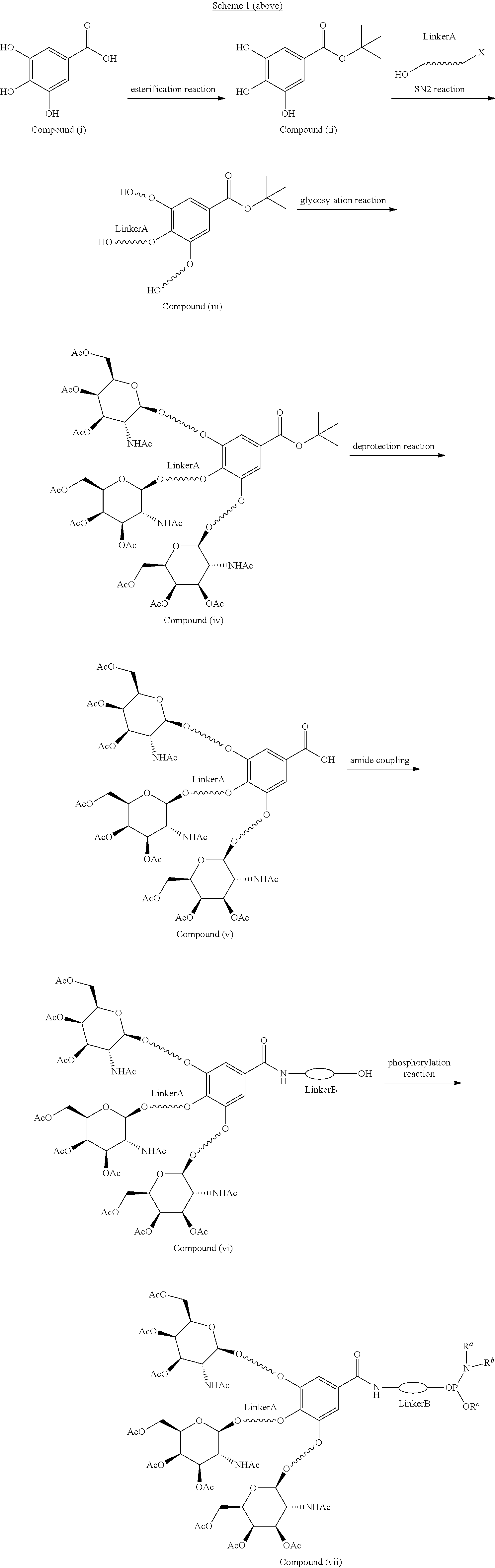

### Synthesis Scheme 2

An embodiment of a method for preparing a compound comprising general Formula 1 is depicted in a synthesis below, identified as “Scheme 2.” Starting materials and intermediates may be purchased from commercial sources, made from known procedures, made using illustrated procedures, or are otherwise illustrated. The order of carrying out the steps of the reaction scheme may be varied. See Examples for more details. For example, in Synthesis Scheme 2, Compound 6 corresponds to “Compound (iv)” illustrated in Synthesis Scheme 1 and Compound 7 corresponds to “Compound (v)” illustrated in Synthesis Scheme 1.

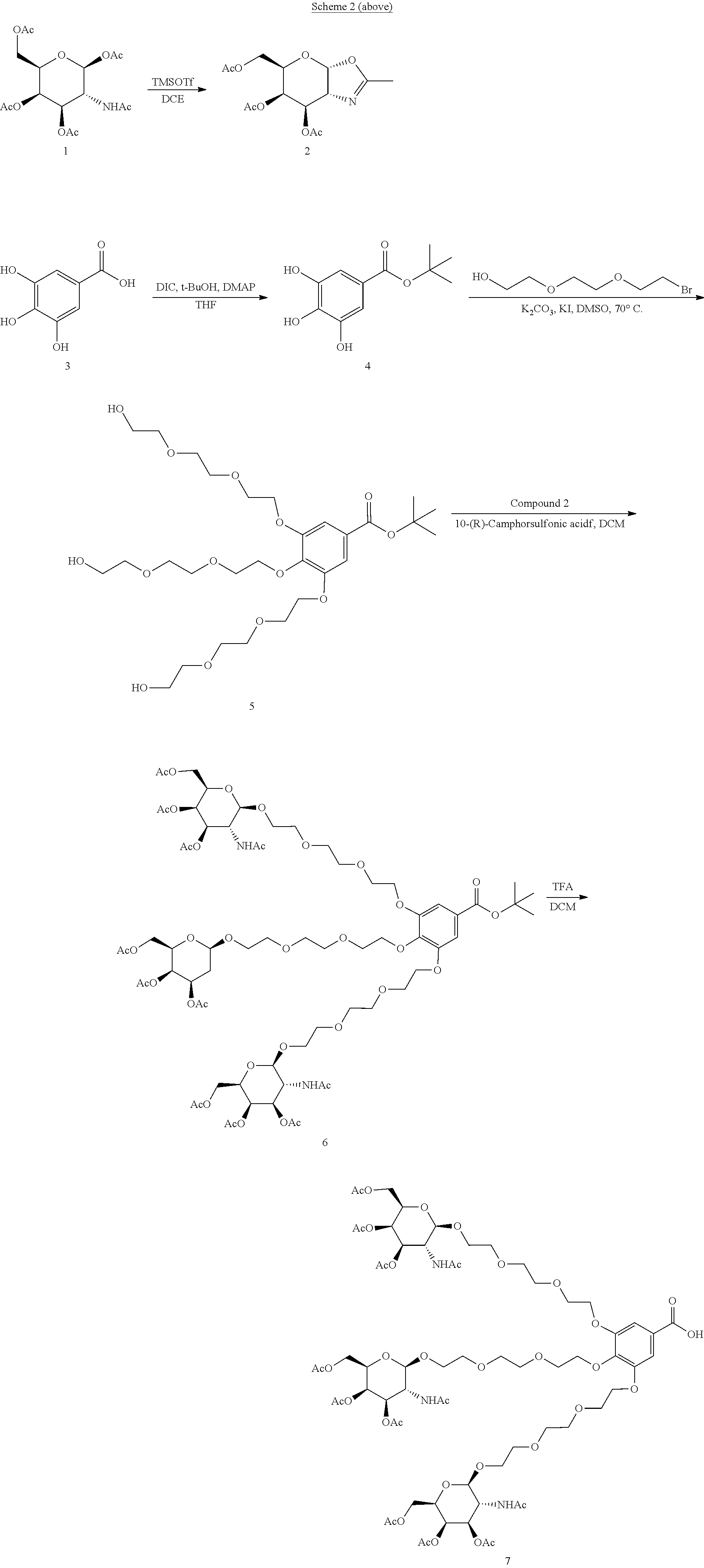

### Synthesis Scheme 3

An embodiment of a method for preparing a compound comprising general Formula 1 is depicted in a synthesis below, identified as “Scheme 3.” Starting materials and intermediates may be purchased from commercial sources, made from known procedures, made using illustrated procedures, or are otherwise illustrated. The order of carrying out the steps of the reaction scheme may be varied. See Examples for further details. In Synthesis Scheme 3, Compound 6′ corresponds to “Compound (iv)” as shown in Synthesis Scheme 1 and Compound 7′ corresponds to “Compound (v)” illustrated in Synthesis Scheme 1.

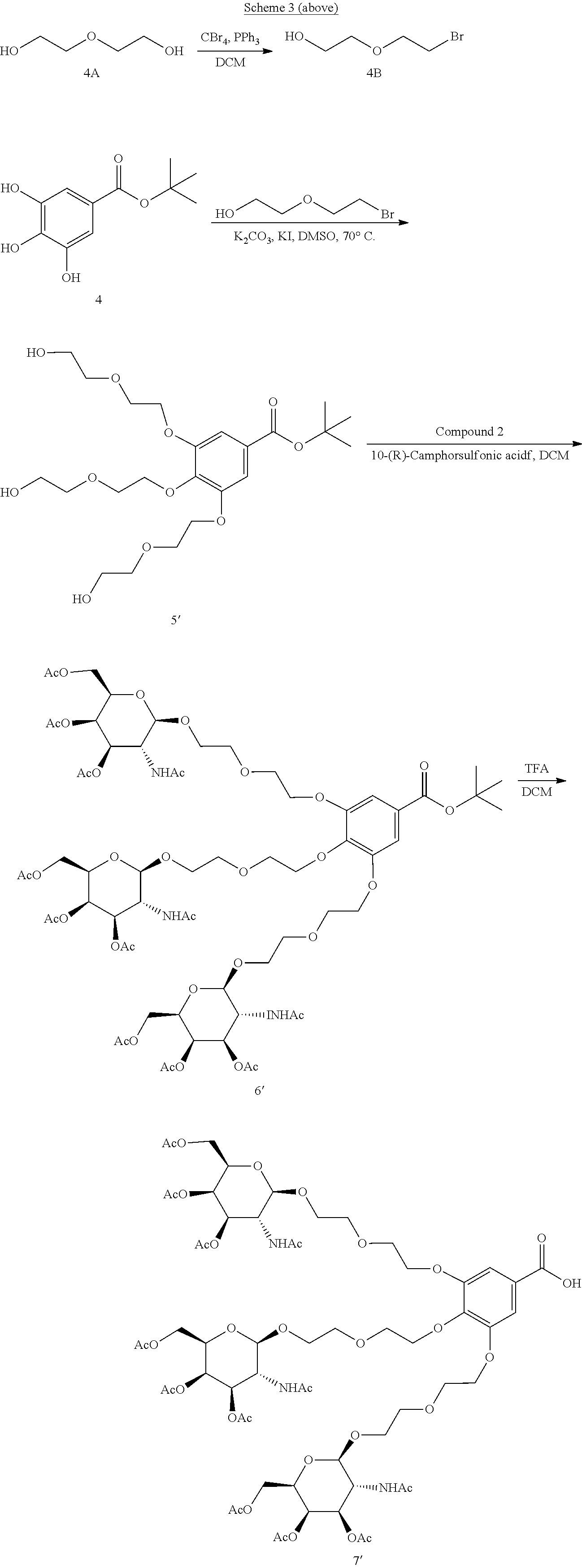

### Synthesis Scheme 4

An embodiment of a method for preparing a compound comprising general Formula 1 and set forth herein as “Compound A” is depicted in a synthesis below, identified as “Scheme 4.” Starting materials and intermediates may be purchased from commercial sources, made from known procedures, made using illustrated procedures, or are otherwise illustrated. The order of carrying out the steps of the reaction scheme may be varied. Synthesis Scheme 4 as shown below begins with Compound 7, which may be prepared as shown in Synthesis Scheme 2. See Examples for more details.

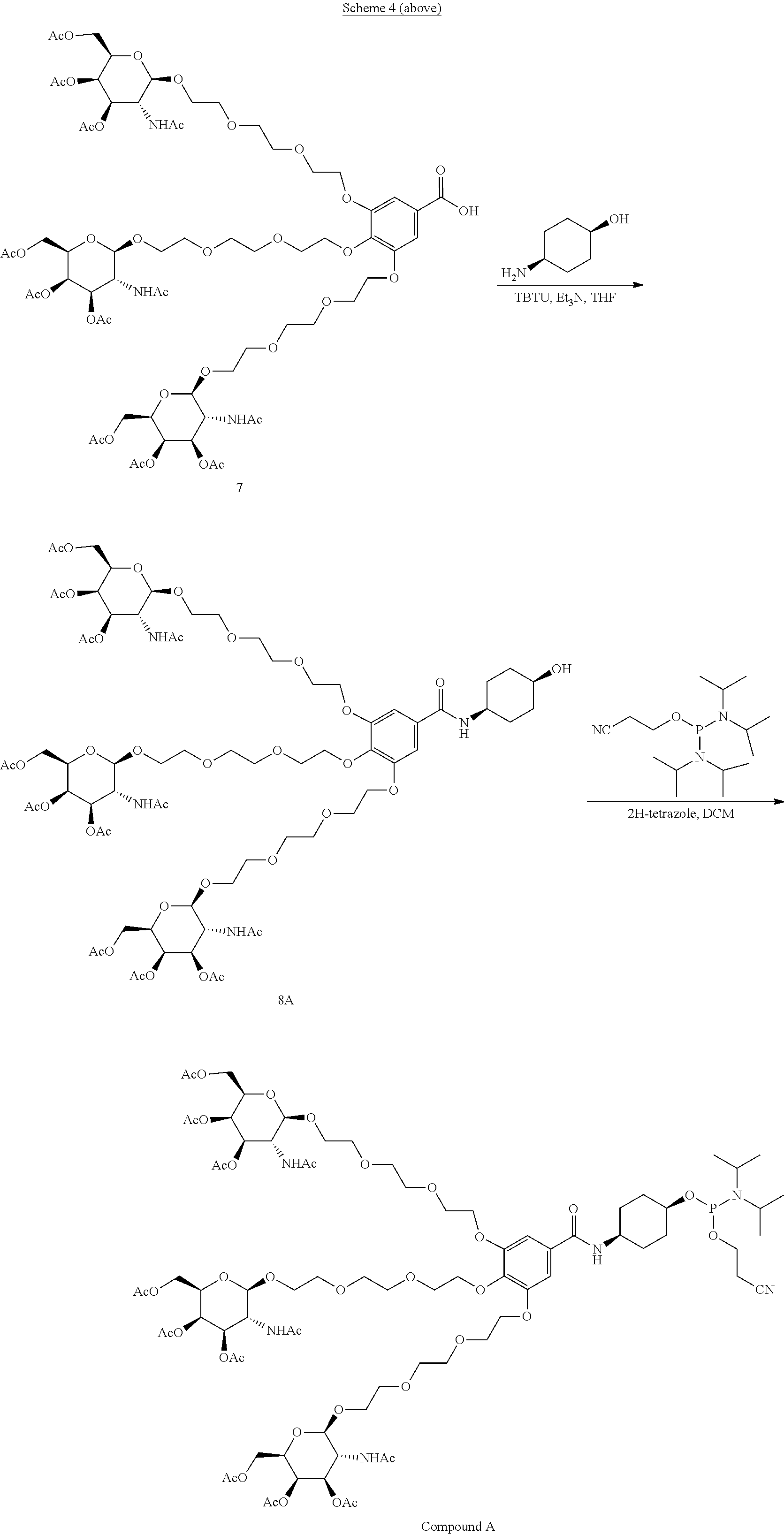

### Certain Elements of Preparation and Use

Certain embodiments of targeting ligand clusters of the invention can be prepared and used to deliver oligonucleotide agents to cells, tissues, and organs. Non-limiting examples of agents that can be delivered include therapeutic agents such as siRNA. Delivery methods using targeting ligand clusters of the invention can be used to deliver siRNAs and other agents conjugated to a target ligand cluster of the invention to in vitro and in vivo cells. Targeting ligand clusters of the invention can be used as a delivery vehicle with which to deliver agents, such as but not limited to agents comprising nucleic acids, to a cell. As used herein, the term “targeting ligand cluster/nucleic acid complex” means a targeting ligand cluster as described herein that is linked to an agent comprising a nucleic acid. In some embodiments of the invention the nucleic acid is an siRNA.

In some aspects of the invention a targeting ligand cluster may be used to deliver an agent to a cell in a subject. Means of administering a targeting ligand cluster/nucleic acid agent to a subject may include art-known methods. As a non-limiting example, a targeting ligand cluster/nucleic acid complex may be locally delivered in vivo by direct injection or by use of an infusion pump. In some aspects of the invention, a targeting ligand cluster/nucleic acid complex is in a pharmaceutical composition and may be referred to as a pharmaceutical agent. In some embodiments, a pharmaceutical agent of the invention is administered to a subject in an amount effective to prevent, modulate the occurrence, treat, or alleviate a symptom of a disease state in the subject.

### Cells and Subjects

As used herein, a subject shall mean a human or vertebrate mammal including but not limited to a dog, cat, horse, goat, cow, sheep, rodent, and primate, e.g., monkey. Thus, the invention can be used to treat diseases or conditions in human and non-human subjects. For instance, methods and compositions of the invention can be used in veterinary applications as well as in human prevention and treatment regimens. In certain embodiments, the subject is a domesticated animal.

The term “subject” refers to any animal. In certain embodiments, the subject is a mammal. In certain embodiments, the subject is a human (e.g., a man, a woman, or a child). The human may be of either sex and may be at any stage of development. In certain embodiments, the subject has been diagnosed with a condition or disease to be treated. In other embodiments, the subject is at risk of developing a condition or disease. In certain embodiments, the subject is an experimental animal (e.g., mouse, rat, rabbit, dog, pig, or primate). The experimental animal may be genetically engineered.

### Assessing Delivery

In certain embodiments of the invention, a targeting ligand cluster/nucleic acid complex of the invention is delivered to and contacted with a cell. In some embodiments of the invention a contacted cell is in culture and in other embodiments a contacted cell is in a subject. Types of cells that may be contacted with a targeting ligand cluster/nucleic acid complex of the invention include, but are not limited to: liver cells, muscle cells, cardiac cells, circulatory cells, neuronal cells, glial cells, fat cells, skin cells, hematopoietic cells, epithelial cells, immune system cells, endocrine cells, exocrine cells, endothelial cells, sperm, oocytes, muscle cells, adipocytes, kidney cells, hepatocytes, or pancreas cells. In some embodiments, the cell contacted with a targeting ligand cluster/nucleic acid complex of the invention is a liver cell.

In some embodiments of the invention, a biological sample may be obtained and assessed for delivery of a nucleic acid using a targeting ligand cluster of the invention. The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

### Administration and Treatment

In certain embodiments of the invention a targeting ligand cluster/nucleic acid complex of the invention can be administered to a subject in a method comprising use of the targeting ligand cluster to deliver the nucleic acid to a cell in the subject. In some embodiments, the nucleic acid is an oligonucleotide, and in some embodiments the oligonucleotide comprises an inhibitor RNA, or siRNA molecule selected to reduce expression of the siRNA's target gene upon delivery. Certain embodiments of the invention include methods of treating a disease or condition associated with expression of a gene in a cell or cells of a subject, wherein the administration of the targeting ligand cluster/nucleic acid complex reduces expression of the gene and treats the disease or condition in the subject. Administration of a targeting ligand cluster/nucleic acid complex of the invention may be done using routine methods.

As used herein the terms “administer,” “administering,” or “administration” refer to implanting, absorbing, ingesting, injecting, inhaling, or otherwise introducing an inventive compound, or a pharmaceutical composition thereof. The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, a treatment may be administered after one or more signs or symptoms of a disease or condition have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. The terms “condition,” “pathological condition,” “disease,” and “disorder” are used interchangeably.

### Dosage

Dosage levels for the medicament and pharmaceutical compositions that may be delivered using a targeting ligand cluster/nucleic acid complex of the present disclosure can be determined by those skilled in the art by routine experimentation. In at least some embodiments, a unit dose may contain between about 0.01 mg/kg and about 100 mg/kg body weight of siRNA. Alternatively, the dose can be from 10 mg/kg to 25 mg/kg body weight, or 1 mg/kg to 10 mg/kg body weight, or 0.05 mg/kg to 5 mg/kg body weight, or 0.1 mg/kg to 5 mg/kg body weight, or 0.1 mg/kg to 1 mg/kg body weight, or 0.1 mg/kg to 0.5 mg/kg body weight, or 0.5 mg/kg to 1 mg/kg body weight. Clinical trials are routinely used to assess dosage levels for therapeutic compositions.

A pharmaceutical composition comprising a targeting ligand cluster of the invention may be a sterile injectable aqueous suspension or solution, or in a lyophilized form. The pharmaceutical compositions and medicaments of the present disclosure may be administered to a subject in a pharmaceutically effective dose.

### Administration Methods

A variety of administration routes for a targeting ligand cluster/nucleic acid complex of the invention are available. The particular delivery mode selected will depend upon the particular condition being treated and the dosage required for therapeutic efficacy. Methods of this invention, generally speaking, may be practiced using any mode of administration that is medically acceptable, meaning any mode that produces effective levels of treatment without causing clinically unacceptable adverse effects. In some embodiments of the invention, a targeting ligand cluster/nucleic acid complex of the invention may be administered via an oral, enteral, mucosal, percutaneous, and/or parenteral route. The term “parenteral” includes subcutaneous, intrathecal, intravenous, intramuscular, intraperitoneal, and intrasternal injection, or infusion techniques. Other routes include but are not limited to nasal (e.g., via a gastro-nasal tube), dermal, vaginal, rectal, and sublingual. Delivery routes of the invention may include intrathecal, intraventricular, or intracranial. In some embodiments of the invention, a targeting ligand cluster/nucleic acid complex of the invention may be placed within a slow release matrix and administered by placement of the matrix in the subject.

A targeting ligand cluster/nucleic acid complex of the invention may be administered in formulations, which may be administered in pharmaceutically acceptable solutions, which may routinely contain pharmaceutically acceptable concentrations of salt, buffering agents, preservatives, compatible carriers, adjuvants, and optionally other therapeutic ingredients. According to methods of the invention, the targeting ligand cluster/nucleic acid complex may be administered in a pharmaceutical composition. In general, a pharmaceutical composition comprises the targeting ligand cluster/nucleic acid complex of the invention and a pharmaceutically-acceptable carrier. Pharmaceutically acceptable carriers are well known to the skilled artisan and may be selected and utilized using routine methods. As used herein, a pharmaceutically-acceptable carrier means a non-toxic material that does not interfere with the effectiveness of the biological activity of the active ingredients, e.g., the ability of the delivered nucleic acid, for example the siRNA to prevent and/or treat a disease or condition to which it is directed.

Pharmaceutically acceptable carriers may include diluents, fillers, salts, buffers, stabilizers, solubilizers and other materials that are well-known in the art. Exemplary pharmaceutically acceptable carriers are described in U.S. Pat. No. 5,211,657 and others are known by those skilled in the art. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention.

The term “pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. The salts can be prepared during the final isolation and purification of the compounds or separately by reacting the appropriate compound in the form of the free base with a suitable acid. Pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable salts include inorganic acids such as hydrochloric acid, hydrobromic acid, sulfuric acid, and phosphoric acid; and organic acids such as oxalic acid, maleic acid, succinic acid, and citric acid.

A targeting ligand cluster/nucleic acid complex of the invention may be administered in a pharmaceutical composition such as those described herein. A pharmaceutical composition of the invention may comprise a targeting ligand cluster/nucleic acid complex of the invention associated with a solvent, usually by a solvolysis reaction. This physical association may include hydrogen bonding. Conventional solvents include water, methanol, ethanol, acetic acid, DMSO, THF, diethyl ether, and the like. The compounds of the invention may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula RxH₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R.0.5H₂O)), and polyhydrates (x is a number greater than 1, e.g., dihydrates (R.2H₂O) and hexahydrates (R.6H₂)).

### Administration

In some embodiments of the invention, a targeting ligand cluster/nucleic acid complex of the invention may be administered directly to a tissue. Direct tissue administration may be achieved by direct injection, or other art-known means. A targeting ligand cluster/nucleic acid complex of the invention may be administered once, or alternatively may be administered in a plurality of administrations. If administered multiple times, a targeting ligand cluster/nucleic acid complex of the invention may be administered via different routes. For example, the first (or the first few) administrations may be made directly into an affected tissue or organ while later administrations may be systemic.

A targeting ligand cluster/nucleic acid complex of the invention, when it is desirable to have it administered systemically, may be formulated for parenteral administration by injection, e.g., by bolus injection or continuous infusion. Formulations for injection may be presented in unit dosage form, e.g., in ampoules or in multi-dose containers, with or without an added preservative. The pharmaceutical compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day may be used as needed to achieve appropriate systemic or local levels of one or more targeting ligand cluster/nucleic acid complexes of the invention, to result in a desired level of the nucleic acid, for example a desired level of the siRNA.

Both non-biodegradable and biodegradable polymeric matrices can be used to deliver one or more targeting ligand cluster/nucleic acid complexes of the invention to a cell and/or subject. In some embodiments, a matrix may be biodegradable. Matrix polymers may be natural or synthetic polymers. A polymer can be selected based on the period of time over which release is desired, generally in the order of a few hours to a year or longer. Typically, release over a period ranging from between a few hours and three to twelve months can be used. The polymer optionally is in the form of a hydrogel that can absorb up to about 90% of its weight in water and further, optionally is cross-linked with multivalent ions or other polymers.

In certain embodiments of the invention, a targeting ligand cluster/nucleic acid complex of the invention may be delivered using the bioerodible implant by way of diffusion, or by degradation of the polymeric matrix. Exemplary synthetic polymers for such use are well known in the art. Biodegradable polymers and non-biodegradable polymers can be used for delivery of one or more of a targeting ligand cluster/nucleic acid complex of the invention using art-known methods. Such methods may also be used to deliver one or more targeting ligand cluster/nucleic acid complexes of the invention for treatment. Additional suitable delivery systems can include time-release, delayed release or sustained-release delivery systems. Such systems can avoid repeated administrations of a targeting ligand cluster/nucleic acid complex of the invention, increasing convenience to the subject and the health-care provider. Many types of release delivery systems are available and known to those of ordinary skill in the art. (See for example: U.S. Pat. Nos. 5,075,109; 4,452,775; 4,675,189; 5,736,152; 3,854,480; 5,133,974; and 5,407,686 (the teaching of each of which is incorporated herein by reference). In addition, pump-based hardware delivery systems can be used, some of which are adapted for implantation.

Use of a long-term sustained release implant may be particularly suitable for prophylactic treatment of subjects and for subjects at risk of developing a recurrent disease or condition to be prevented and/or treated with an siRNA delivered using a targeting ligand cluster of the invention. Long-term release, as used herein, means that the implant is constructed and arranged to delivery therapeutic levels of the active ingredient for at least 30 days, 60 days, 90 days or longer. Long-term sustained release implants are well-known to those of ordinary skill in the art and include some of the release systems described above.

Therapeutic formulations of one or more targeting ligand cluster/nucleic acid complexes of the invention may be prepared for storage by mixing the targeting ligand cluster/nucleic acid complex having the desired degree of purity with optional pharmaceutically acceptable carriers, excipients or stabilizers [Remington's Pharmaceutical Sciences 21^{st} edition, (2006)], in the form of lyophilized formulations or aqueous solutions. Acceptable carriers, excipients, or stabilizers are nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

The siRNA conjugates of the present disclosure (also referred to herein as targeting ligand cluster/nucleic acid complexes) may be formulated as pharmaceutical compositions. The pharmaceutical compositions may be used as medicaments, alone or in combination with other agents. The siRNA conjugates of the present disclosure can also be administered in combination with other therapeutic compounds, either administrated separately or simultaneously (e.g., as a combined unit dose). In at least some embodiments, the present disclosure includes a pharmaceutical composition comprising one or more siRNA conjugates according to the present disclosure in a physiologically/pharmaceutically acceptable excipient, such as a stabilizer, preservative, diluent, buffer, and the like.

A pharmaceutical composition of the invention may be administered alone, in combination with each other, and/or in combination with other drug therapies, or other treatment regimens that are administered to subjects with a disease or condition. Pharmaceutical compositions used in the embodiments of the invention preferably are sterile and contain an effective amount of a targeting ligand cluster/nucleic acid complex to prevent or treat a disease or condition, to which the nucleic acid, for example the siRNA is directed.

The dose or doses of a pharmaceutical composition of the invention that are sufficient to treat a disease or condition when administered to a subject can be chosen in accordance with different parameters, in particular in accordance with the mode of administration used and the state of the subject. Other factors may include the desired period of treatment. In the event that a response in a subject is insufficient at the initial doses applied, higher doses (or effectively higher doses by a different, more localized delivery route) may be employed to the extent that patient tolerance permits. In some embodiments of the invention, dosing is used that has been determined using routine means such as in clinical trials.

### EXAMPLES

In order that the invention described herein may be more fully understood, the following examples are set forth. The examples described in this application are offered to illustrate the methods and compositions provided herein and are not to be construed in any way as limiting their scope.

### Example 1

### Scheme 1 Synthesis of an Embodiment of a Targeting Ligand Cluster

An embodiment of a method for preparing a targeting ligand cluster compound comprising general Formula 2 is depicted in a synthesis below, identified as “Scheme 1.” Starting materials and intermediates may be purchased from commercial sources, made from known procedures, or are otherwise illustrated. The order of carrying out the steps of the reaction scheme may be varied. The following method has been used to prepare a targeting ligand cluster compound comprising general Formula 2.

General synthetic Materials and Methods

Starting from Gallic acid (Compound (i) in Scheme 1), tert-Butylester of gallic acid [Compound (ii)] was synthesized using a procedure described in Leiro, V.; et al. J. Mater. Chem. B, 2017, 5, 4901, the content of which is incorporated herein by reference in its entirety.

Compound (iii) can be synthesized by reacting Compound (ii) and a Linker A derivative with a suitable leaving group under a standard SN2 reaction condition (for example K₂CO₃ as the base in presence of a catalytic amount of KI and in an aprotic solvent). Compound (iv) can be prepared by treating Compound (iii) with a glycosylation precursor derived from GalNAc (for example (3aR,5R,6R,7R,7aR)-5-(acetoxymethyl)-2-methyl-3a,6,7,7a-tetrahydro-5H-pyrano[3,2-d]oxazole-6,7-diyl diacetate) in the presence of a Lewis or Bronsted acid (for example 10-(R)-Camphorsulfonic Acid).

Deprotection of tBu ester group can be carried out by treating with trifluoroacetic acid (TFA) or formic acid without affecting GalNAc moiety. Thus, treating Compound (vi) with an acid (TFA or formic acid) may afford Compound (v).

An amide coupling reaction between Compound (v) and an amino alcohol (LinkerB) may produce compound (vi).

Finally, phosphoramide Compound (vii) can be synthesized by treating Compound (vi) with 2-Cyanoethyl N,N-diisopropylchlorophosphoramidite and a catalytic amount of 1H-tetrazole. Compound (vii) can be used for synthesis of a GalNAc ligand cluster conjugated oligonucleotide under standard solid phase oligonucleotide synthesis conditions.

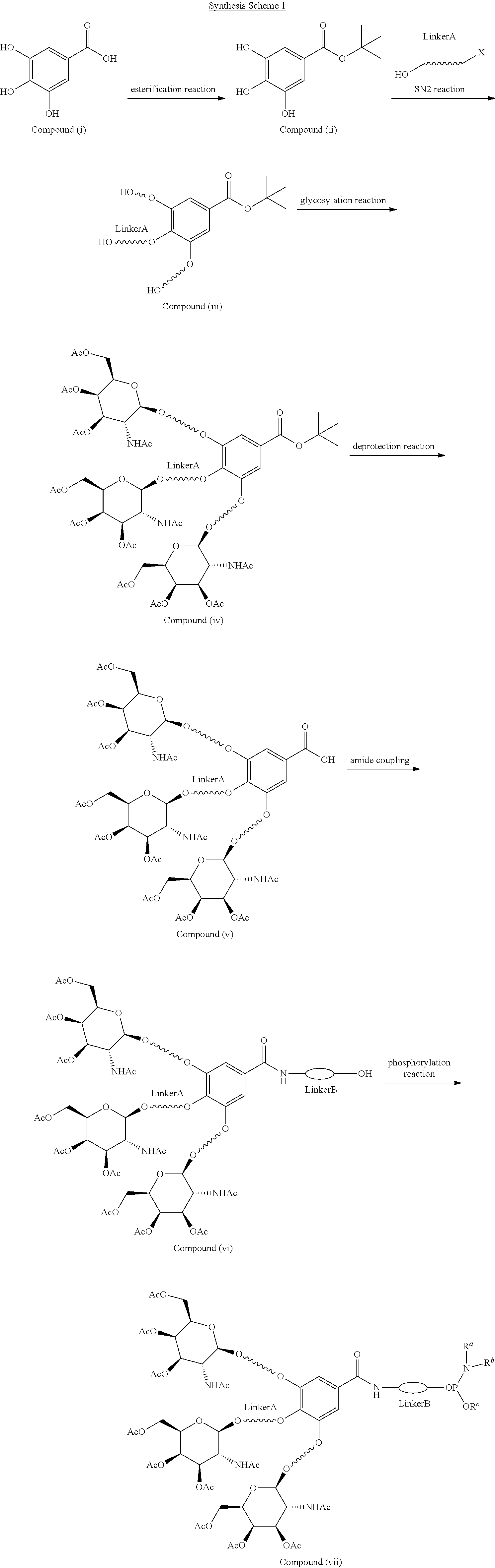

### Example 2

### Scheme 2—Synthesis and Characterization of an Embodiment of a Targeting Ligand Cluster

An embodiment of a method for preparing a compound comprising general Formula 1 is depicted in a synthesis below, identified as “Scheme 2.” Starting materials and intermediates may be purchased from commercial sources, made from known procedures, or are otherwise illustrated. The order of carrying out the steps of the reaction scheme may be varied. The following method has been used to prepare a targeting ligand cluster compound comprising general Formula 1.

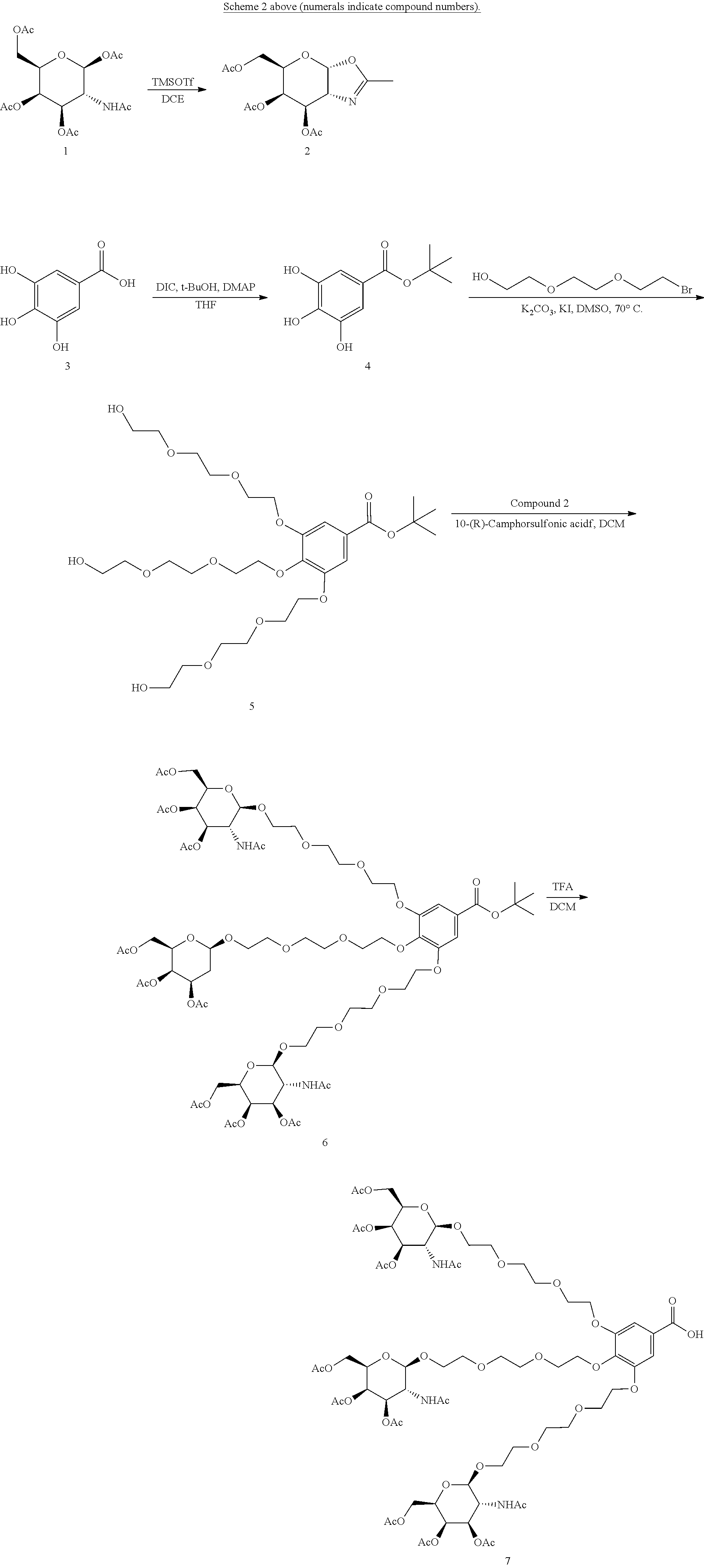

### Example 3

### Synthesis Scheme 3—Synthesis and Characterization of an Embodiment of a Targeting Ligand Cluster

An embodiment of a method for preparing a compound comprising general Formula 1 is depicted in a synthesis below, identified as “Scheme 3.” Starting materials and intermediates may be purchased from commercial sources, made from known procedures, or are otherwise illustrated. The order of carrying out the steps of the reaction scheme may be varied. The following method has been used to prepare a targeting ligand cluster compound comprising general Formula 1.

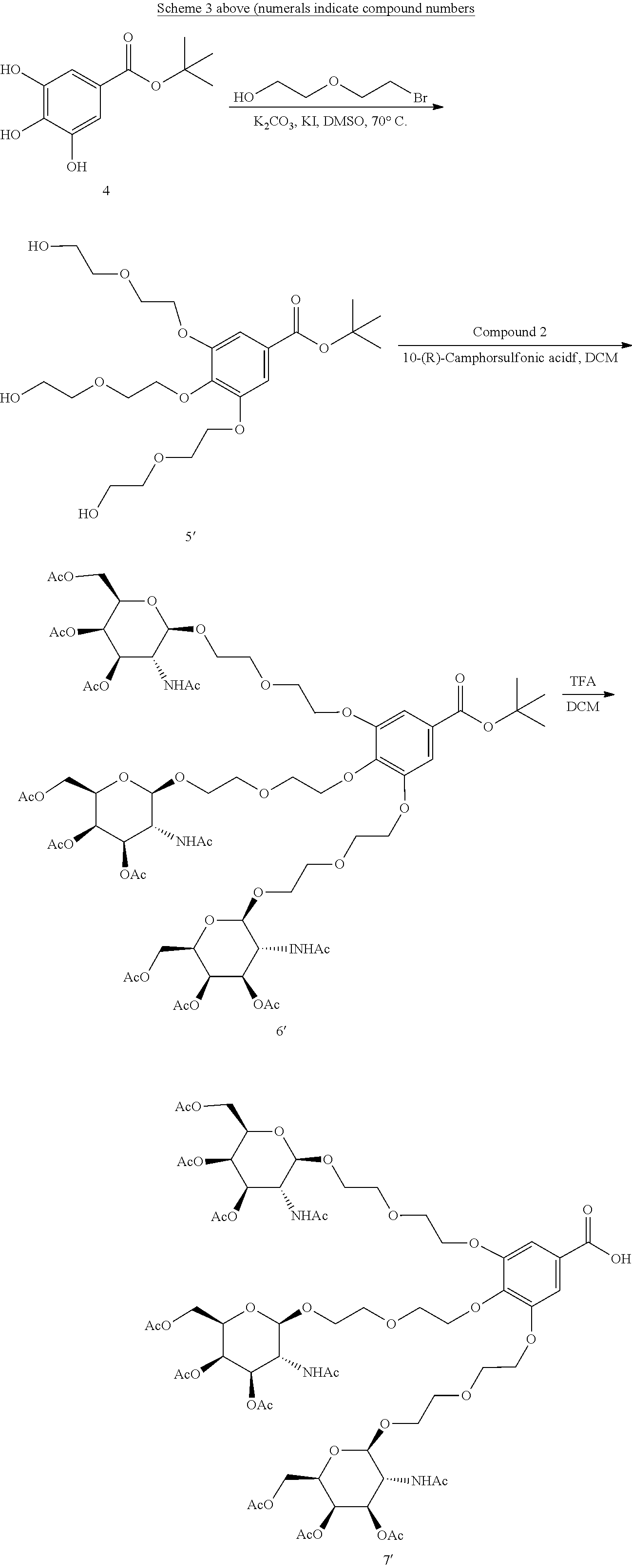

### Example 4

### Synthesis Scheme 4—Synthesis and Characterization of an Embodiment of a Targeting Ligand Cluster

The following synthesis scheme was used to prepare an embodiment of Compound “A” compound, which comprises general Formula 1. The synthesis Scheme 4 is identified as “Scheme 4.” Starting materials and intermediates may be purchased from commercial sources, made from known procedures, or are otherwise illustrated. The order of carrying out the steps of the reaction scheme may be varied. The following method has been used to prepare a targeting ligand cluster compound comprising general Formula 1.

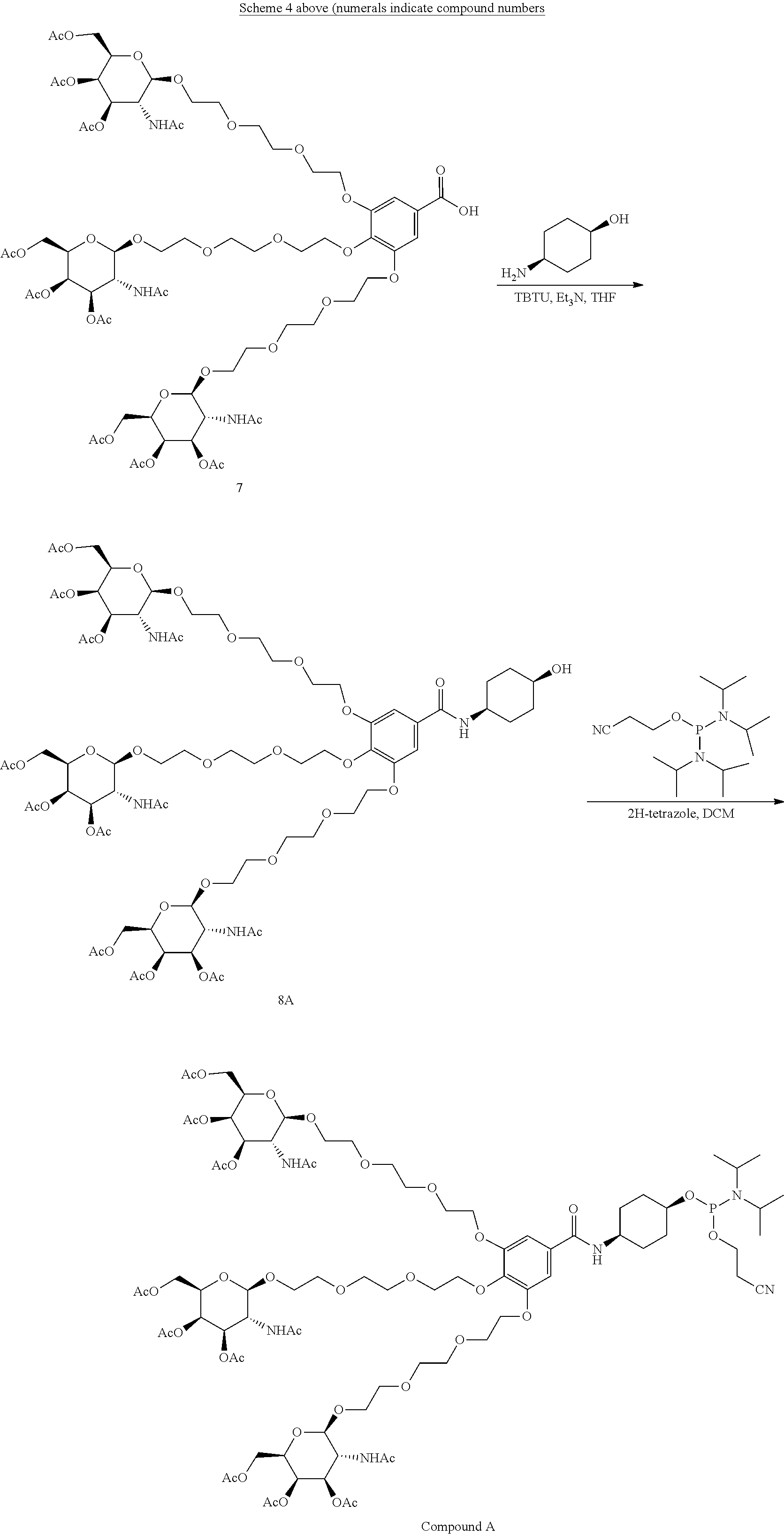

### Example 5

### Preparation of Compound 2

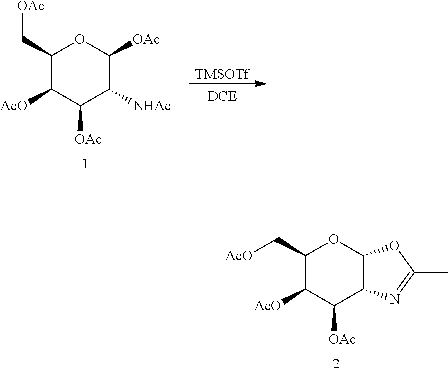 [SMILES: "TMSOTf" ; CC(=O)N[C@H]1[C@H](OC(C)=O)O[C@H](COC(C)=O)[C@H](OC(C)=O)[C@@H]1OC(C)=O ; CC(=O)OC[C@H]1O[C@@H]2OC(C)=N[C@@H]2[C@@H](OC(C)=O)[C@H]1OC(C)=O]

To a solution of compound 1 (25.0 g, 64.2 mmol) in DCE (250 mL) was added TMSOTf (17.1 g, 77.1 mmol, 13.9 mL) dropwise at 0° C. under N₂ atmosphere. The mixture was stirred at 20° C. for 40 hr. TLC indicated little compound 1 remaining and one new spot formed (dichloromethane:methyl alcohol=10:1, R_{f}=0.51). The reaction was quenched by the addition of NaHCO₃ (1000 mL), extracted with DCM (1000 mL*3). The organic phase was dried with anhydrous Na₂SO₄ and concentrated under reduced pressure to give a residue. The residue was purified by silica gel column chromatography (dichloromethane/methanol=100/1 to 60/1) to give compound 2. This reaction was repeated 3 more times and final products from these 4 runs were combined to give total 45.0 gram of compound 2 (137 mmol, 53.2% yield) as a pale yellow oil. ¹H NMR (400 MHz, CDCl₃): δ ppm 5.97 (d, J=7.03 Hz, 1H), 5.43 (t, J=3.01 Hz, 1H), 4.89 (dd, J=7.40, 3.39 Hz, 1H), 4.18-4.24 (m, 1H), 4.14-4.18 (m, 1H), 4.05-4.11 (m, 1H), 3.97 (td, J=7.15, 1.25 Hz, 1H), 2.08-2.11 (m, 3H), 2.04 (s, 6H), 2.03 (d, J=1.25 Hz, 3H).

### Example 6

### Preparation of Compound 4

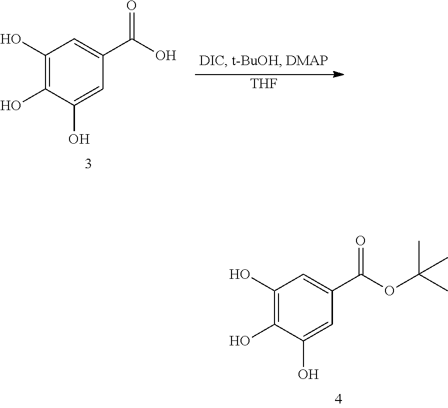 [SMILES: O=C(O)c1cc(O)c(O)c(O)c1 ; CC(C)(C)OC(=O)c1cc(O)c(O)c(O)c1 ; "THF"]

To a solution of compound 3 (20.0 g, 118 mmol, 47.6 mL), 2-methylpropan-2-ol (17.4 g, 235 mmol, 22.5 mL) in THF (200 mL) was added DIC (22.3 g, 176 mmol, 27.3 mL) and stirred for 1 hr at 0° C. Then DMAP (1.44 g, 11.8 mmol) was added to the mixture and stirred for another 17 hr at 20° C. TLC (ethyl acetate:petroleum ether=1:1, R_{f}=0.25) indicated most of compound 3 was consumed, and one major new spot with lower polarity was detected. The reaction mixture was neutralized by addition HCl (1N, 100 mL), and then extracted with EA (500 mL*3). The combined organic layers were dried over anhydrous Na₂SO₄, filtered and concentrated under reduced pressure to give a residue. The residue was purified by flash silica gel chromatography (ISCO®; 330 g SepaFlash® Silica Flash Column, Eluent of 0˜100% Ethyl acetate/Petroleum ether gradient @ 100 mL/min) to give Compound 4 (9.00 g, 39.8 mmol, 33.8% yield) as a pale yellow liquid. ¹H NMR (400 MHz, DMSO-d6): δ ppm 9.18 (br s, 2H), 8.83 (br s, 1H), 6.88 (s, 2H), 1.49 (s, 9H).

### Example 7

### Preparation of Compound 5

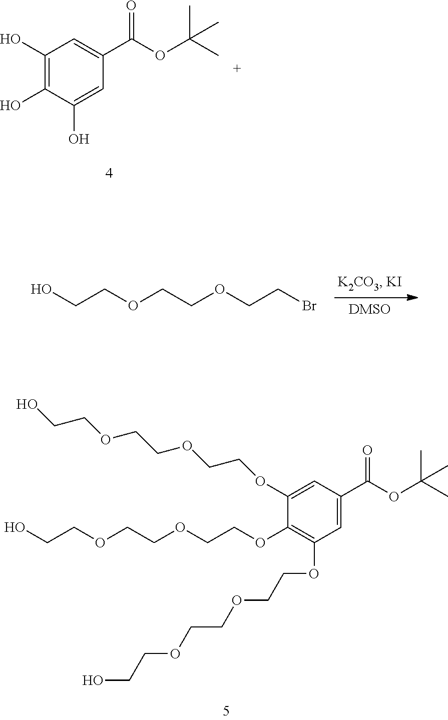 [SMILES: CC(C)(C)OC(=O)c1cc(O)c(O)c(O)c1 ; OCCOCCOCCBr ; CC(C)(C)OC(=O)c1cc(OCCOCCOCCO)c(OCCOCCOCCO)c(OCCOCCOCCO)c1]

To a solution of Compound 4 (2.00 g, 8.84 mmol) in DMSO (60.0 mL) was added K₂C₀₃ (4.89 g, 35.4 mmol), and KI (440 mg, 2.65 mmol). Reaction mixture was heated to 70° C. Then 2-(2-(2-bromoethoxy)ethoxy)ethan-1-ol (7.53 g, 35.4 mmol) was added to the mixture and the mixture was stirred at 70° C. for 4 hrs under N₂ atmosphere. LC-MS showed one main peak with desired m/z (Calculated MW: 622.70, observed m/z: 567.2 [(M-t-Bu)+H]⁺, 640.3 [(M+H₂O)+H]⁺) was detected. The reaction mixture was purified by prep-HPLC (neutral condition) to give compound 5 (3.50 g, 5.62 mmol, 63.6% yield) as a brown oil. ¹H NMR (400 MHz, DMSO-d6): δ ppm 7.17 (s, 2H), 4.58 (t, J=5.44 Hz, 3H), 4.08-4.16 (m, 6H), 3.73-3.78 (m, 4H), 3.65-3.69 (m, 2H), 3.58-3.63 (m, 4H), 3.52-3.57 (m, 6H), 3.45-3.51 (m, 8H), 3.39-3.43 (m, 6H), 1.53 (s, 9H).

### Example 8

### Preparation of Compound 6

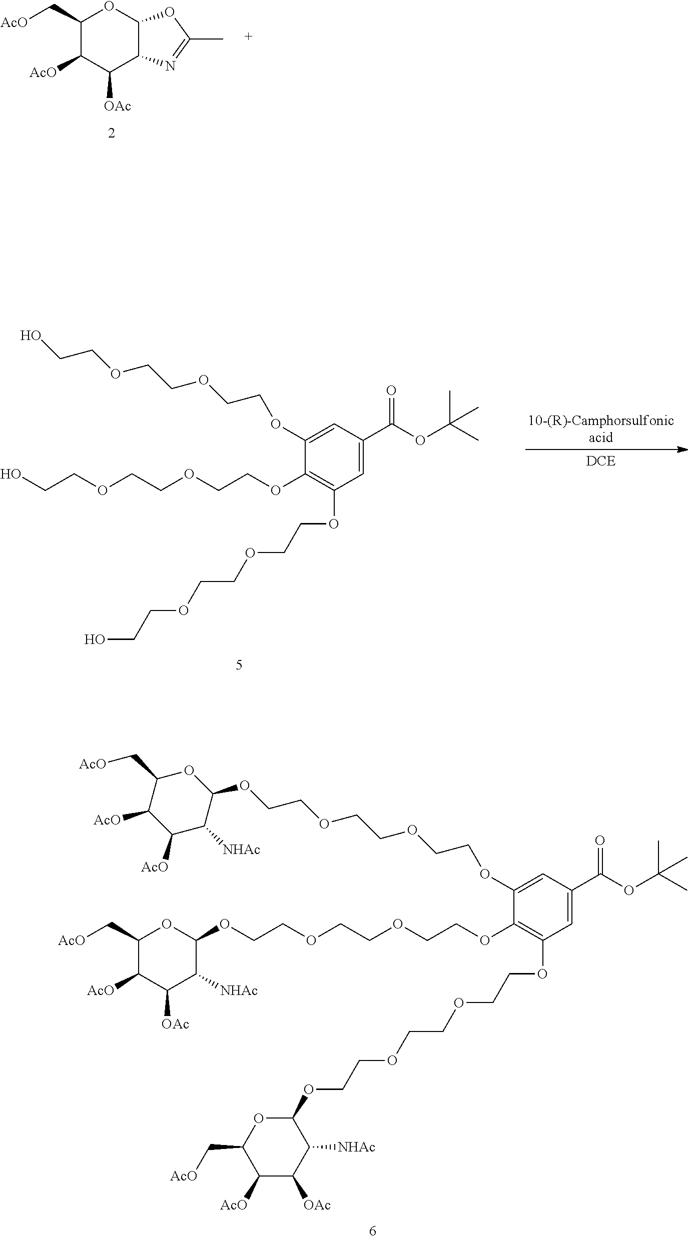 [SMILES: CC(=O)OC[C@H]1O[C@@H]2OC(C)=N[C@@H]2[C@@H](OC(C)=O)[C@H]1OC(C)=O ; CC(C)(C)OC(=O)c1cc(OCCOCCOCCO)c(OCCOCCOCCO)c(OCCOCCOCCO)c1 ; CC(=O)N[C@H]1[C@H](OCCOCCOCCOc2cc(C(=O)OC(C)(C)C)cc(OCCOCCOCCO[C@@H]3O[C@H](COC(C)=O)[C@H](OC(C)=O)[C@H](OC(C)=O)[C@H]3NC(C)=O)c2OCCOCCOCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

To a solution of compound 2 (9.52 g, 28.9 mmol) in anhydrous DCE (150 mL) was stirred with 4 Å molecular sieves for 5 min at 20° C. Then compound 5 (4.50 g, 7.23 mmol) was added and stirring was continued for 30 min. [(1R,4S)-7,7-dimethyl-2-oxo-norbornan-1-yl]methanesulfonic acid (6.04 g, 26.02 mmol, 3.6 eq) was added dropwise over 10 min under N₂ atmosphere. The mixture was stirred at 50° C. for 2 hr. LC-MS showed compound 5 was consumed completely and one main peak with desired m/z (Calculated MW: 1610.61, observed m/z: 805.9 [M/2+H]⁺, 1611.5 [M+H]⁺) was detected. The reaction mixture was filtered through diatomite. The filtrate was quenched by the addition of NaHCO₃ (300 mL), extracted with DCM (300 mL*3). The organic phase was dried with anhydrous Na₂SO₄ and concentrated under reduced pressure to give a residue. The residue was purified by flash silica gel chromatography (ISCO®; 330 g SepaFlash® Silica Flash Column, Eluent of 0˜10% methanol/dichloromethane @ 100 mL/min) to give compound 6 (10.3 g, 6.40 mmol, 88.5% yield) as a pale yellow solid.

### Example 9

### Preparation of Compound 7

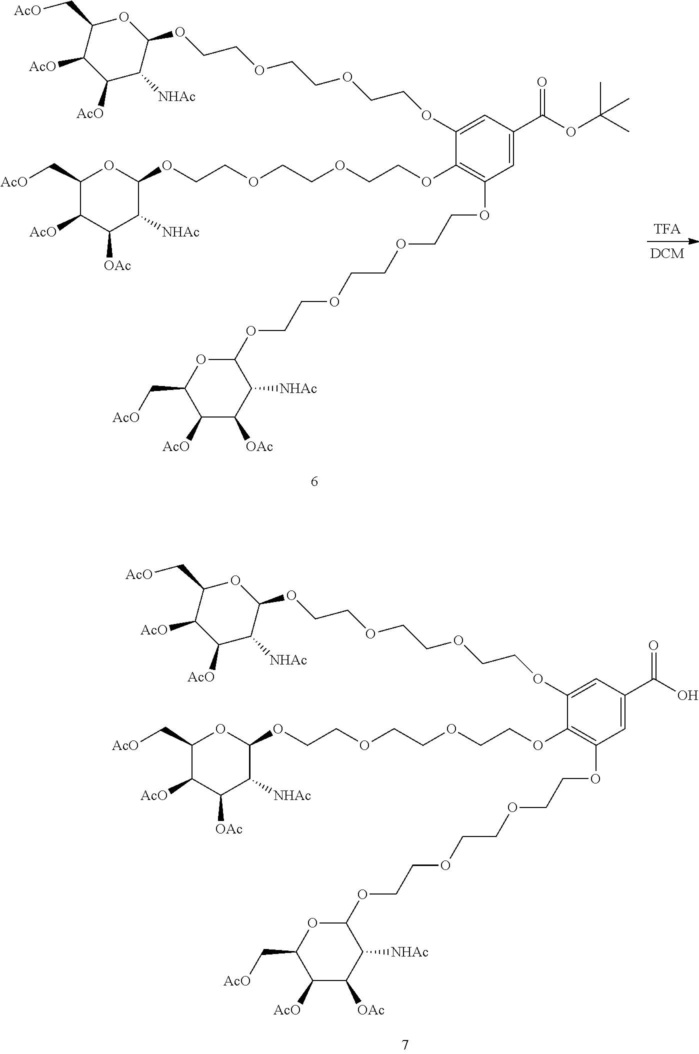 [SMILES: CC(=O)N[C@H]1[C@H](OCCOCCOCCOc2cc(C(=O)OC(C)(C)C)cc(OCCOCCOCCOC3O[C@H](COC(C)=O)[C@H](OC(C)=O)[C@H](OC(C)=O)[C@H]3NC(C)=O)c2OCCOCCOCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O ; CC(=O)N[C@H]1[C@H](OCCOCCOCCOc2cc(C(=O)O)cc(OCCOCCOCCOC3O[C@H](COC(C)=O)[C@H](OC(C)=O)[C@H](OC(C)=O)[C@H]3NC(C)=O)c2OCCOCCOCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

To a solution of Compound 6 (3.43 g, 2.13 mmol) in DCM (17.5 mL) was added TFA (27.0 g, 236 mmol, 17.5 mL). The mixture was stirred at 20° C. for 1 hr. LC-MS showed Compound 6 was consumed completely and one main peak with desired m/z (Calculated MW: 1554.50, observed m/z: 778.4 [M/2+H]⁺) was detected. The reaction mixture was concentrated under reduced pressure to give a residue. The residue was purified by prep-HPLC (A: 0.075% TFA in H₂O, B: ACN) to give compound 7 (4.80 g, 3.09 mmol, 48.3% yield) as a white solid.

### Example 10

### Preparation of Compound 8A

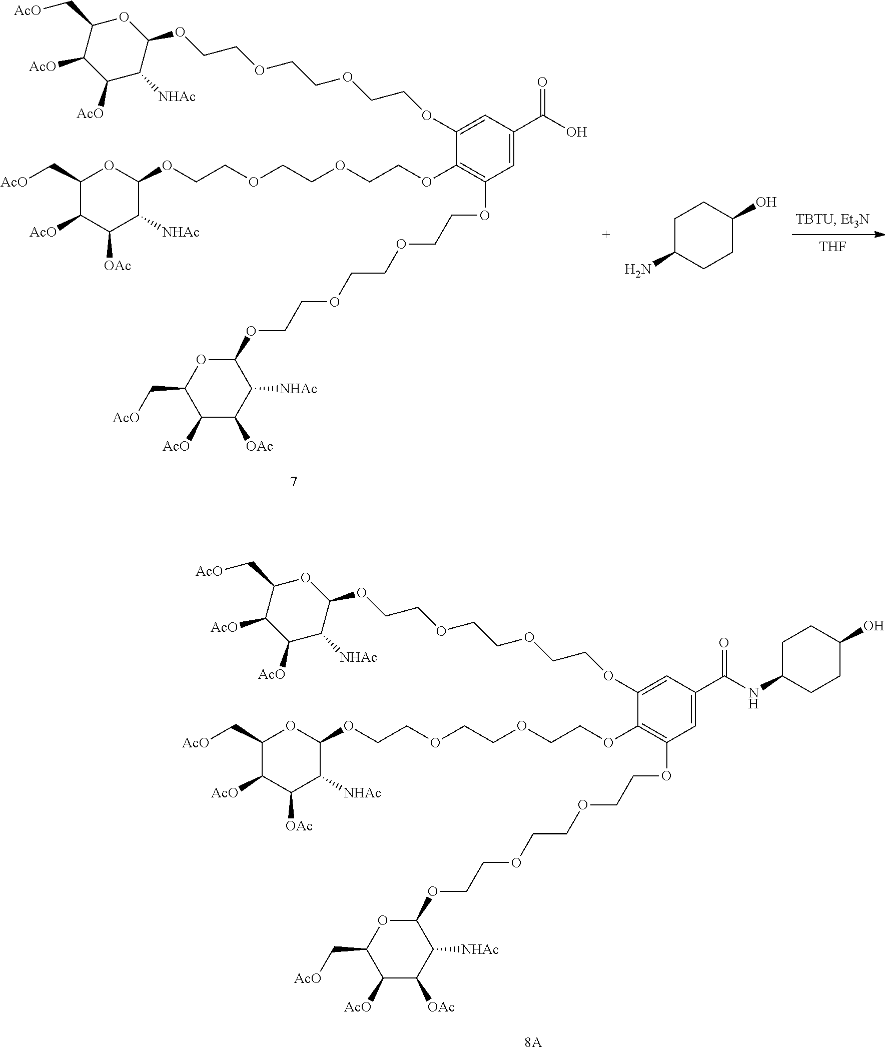

To a solution of compound 7 (500 mg, 322 μmol) in THF (5.00 mL) was added Et₃N (65.1 mg, 643 μmol, 89.5 μL). Then TBTU (103 mg, 322 μmol) and 4-aminocyclohexanol (37.1 mg, 322 μmol) were added to the mixture. The mixture was stirred at 20° C. for 1 hr under N₂ atmosphere. LC-MS showed compound 7 was consumed completely and one main peak with desired m/z (Calculated MW: 1651.66, observed m/z: 826.5 [M/2+H]⁺, 1652.5 [M+H]⁺) was detected. The mixture was dissolved in DCM (100 mL), washed with HCl (1 N, 2*50 mL), organic phase was washed with saturated solution of NaHCO₃ (2*50 mL) and water (3*50 mL), then concentrated under reduced pressure to give a residue. The residue was purified by prep-HPLC (A: 0.075% TFA in H₂O, B: ACN) to give compound 8A (420 mg, 254 μmol, 79.1% yield) as a white solid.

### Example 11

### Preparation of Ligand A

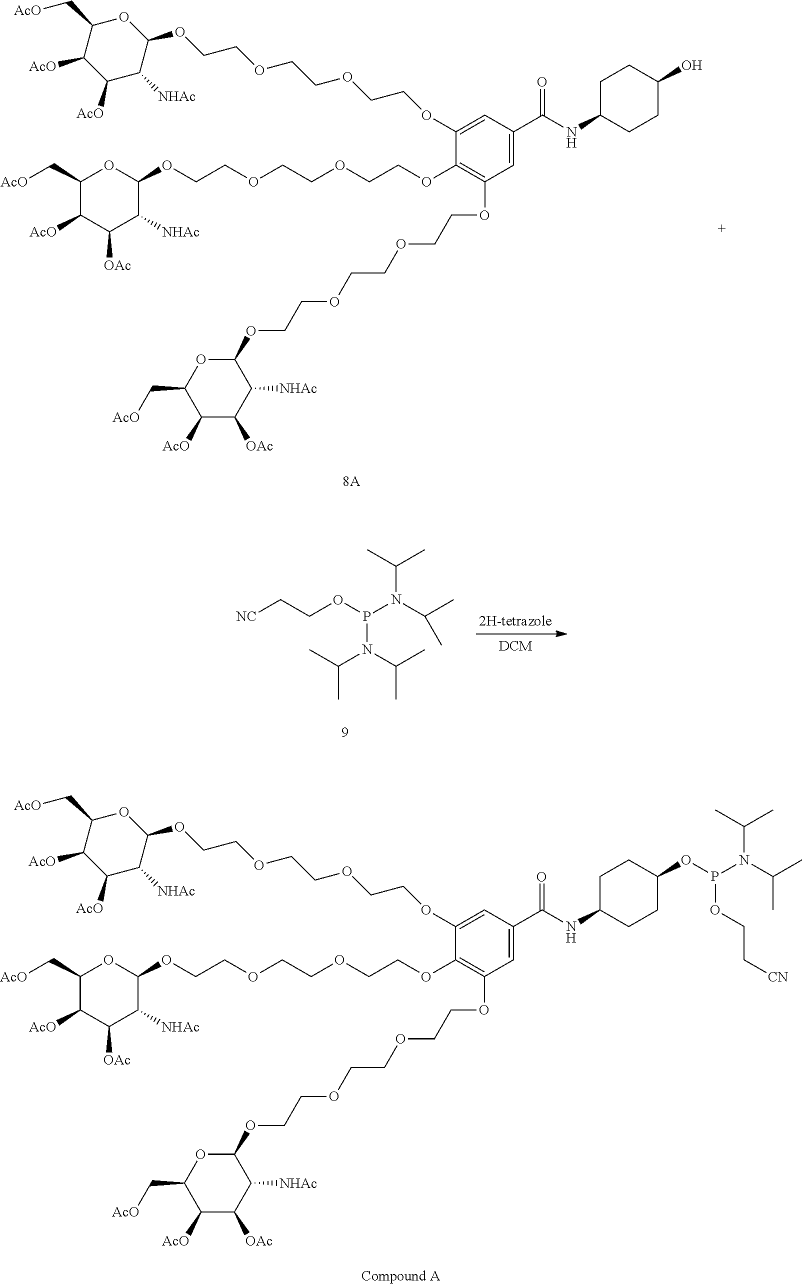

Reaction preparation: Compound 8A was dried 5 times with anhydrous MeCN (azeotropic distillation). MeCN and DCM were dried with spherical 4 Å molecular sieve overnight.

To a solution of Compound 8A (420 mg, 254 μmol) in DCM (4.00 mL) at 0° C. was added Compound 9 (153 mg, 509 μmol, 162 μL) and 2H-tetrazole (0.45 M, 622 μL) dropwise under N₂ atmosphere. Then the mixture was stirred at 0-15° C. for 1 hr. TLC (dichloromethane:methyl alcohol=10:1, R_{f}=0.53) indicated Compound 8A was consumed completely and one new spot formed. The mixture was cooled to −20˜−10° C., then poured into sat. NaHCO₃ (8 mL) slowly at 0-5° C. The resulting mixture was extracted with DCM (15 mL*3), then the organic layer was washed with brine (15 mL), dried over Na₂SO₄, filtered and concentrated while keeping temperature below 20° C. The residue was dissolved in DCM (2 mL), added dropwise to a stirred 20 mL MTBE (−10° C.) at room temperature. Resulting mixture was stirred and filtered. Solid was washed with MTBE (10 mL*3), dried in high vacuum. This purification procedure was repeated two more times to afford Ligand A (210 mg, 113 μmol, 44.6% yield) as a white solid. ¹H NMR (400 MHz, DMSO-d6): δ ppm 8.15 (br d, J=7.78 Hz, 1H), 7.79 (d, J=9.29 Hz, 3H), 7.17 (s, 2H), 5.21 (d, J=3.26 Hz, 3H), 4.97 (dd, J=11.17, 3.39 Hz, 3H), 4.55 (d, J=8.53 Hz, 3H), 4.14 (br t, J=4.52 Hz, 4H), 3.98-4.08 (m, 12H), 3.83-3.92 (m, 4H), 3.64-3.82 (m, 13H), 3.45-3.63 (m, 24H), 2.77 (t, J=5.77 Hz, 2H), 2.10 (s, 9H), 1.99 (s, 9H), 1.89 (s, 9H), 1.76 (d, J=1.25 Hz, 9H), 1.54-1.74 (m, 6H), 1.16 (d, J=6.78 Hz, 11H). ³¹P NMR: δ ppm 145.70.

### Example 12

### Preparation of Compound 8B

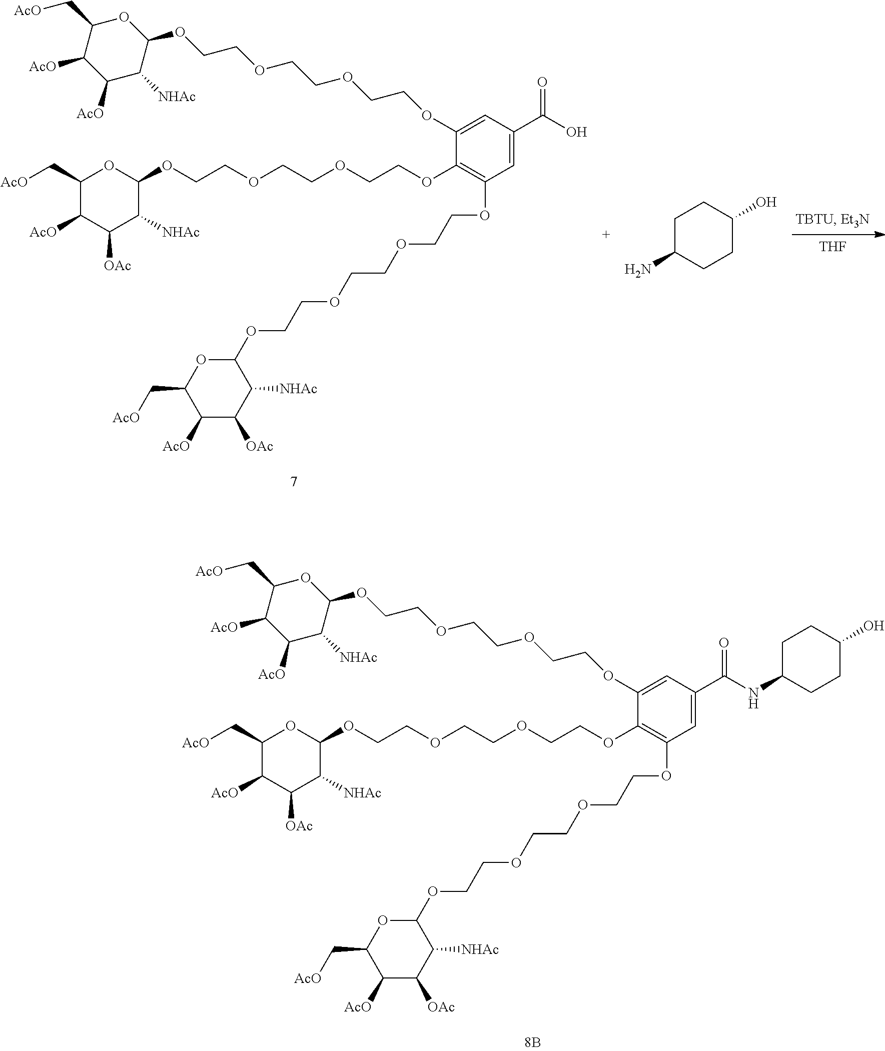

To a solution of compound 7 (500 mg, 322 μmol) in THF (5.00 mL) was added Et₃N (65.1 mg, 643 μmol, 89.5 μL). Then TBTU (103 mg, 322 μmol) and 4-aminocyclohexanol (37.1 mg, 322 μmol) were added to the mixture. The mixture was stirred at 20° C. for 1 hr under N₂ atmosphere. LC-MS showed Compound 7 was consumed completely and one main peak with desired m/z (Calculated MW: 1651.55, observed m/z: 826.5 [M/2+H]⁺, 1652.6 [M+H]⁺) was detected. The mixture was dissolved in DCM (100 mL), washed with HCl (1 N, 2*50 mL), organic phase was washed with saturated solution of NaHCO₃ (2*50 mL) and water (3*50 mL), then concentrated under reduced pressure to give a residue. The residue was purified by prep-HPLC (A: 0.075% TFA in H₂O, B: ACN) to give Compound 8B (395 mg, 239 μmol, 74.4% yield) as a white solid.

### Example 13

### Preparation of Ligand B

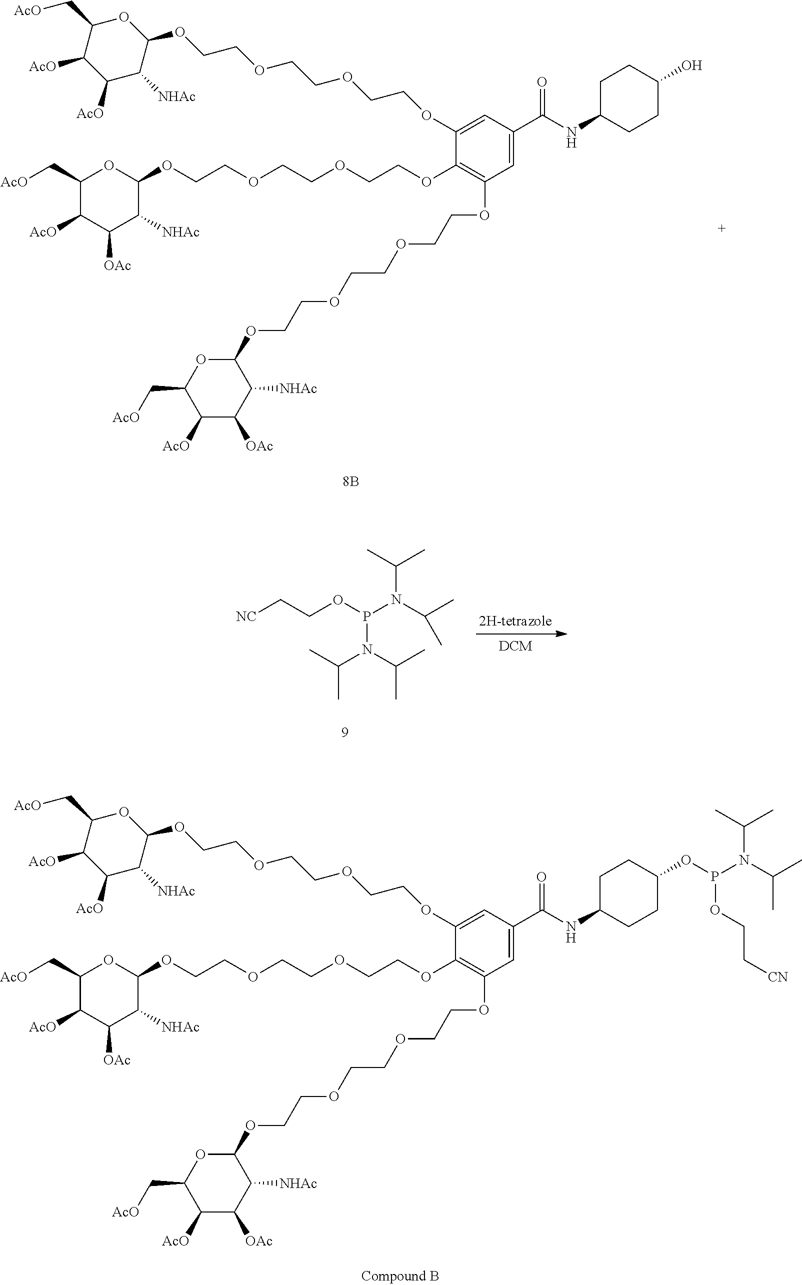

Reaction preparation: Compound 8B was dried 5 times with anhydrous MeCN (azeotropic distillation). MeCN and DCM were dried with spherical 4 Å molecular sieve overnight.

To a solution of Compound 8B (288 mg, 174 μmol) in DCM (3.00 mL) at 0° C. was added Compound 9 (105 mg, 349 μmol, 111 μL) and 2H-tetrazole (0.45 M, 426 μL) dropwise under N₂ atmosphere. Then the mixture was stirred at 0-15° C. for 1 hr. TLC (dichloromethane:methyl alcohol=10:1, R_{f}=0.51) indicated Compound 8B was consumed completely and one new spot formed. The mixture was cooled to −20˜−10° C., then poured into sat. NaHCO₃ (8 mL) slowly at 0-5° C., washed with DCM (10 mL*2), the aqueous was extracted with DCM (15 mL) after separated, then the organic layer was washed with brine (15 mL), dried over Na₂SO₄, filtered and concentrated while keeping temperature below 20° C. The residue was dissolved in DCM (2 mL), added dropwise to a stirred 20 mL MTBE (−10° C.) at room temperature, stirred and filtered, washed with MTBE (10 mL*3), dried in high vacuum. This purification procedure was repeated two more times to afford Ligand B (235 mg, 127 μmol, 72.8% yield) as a white solid. ¹H NMR (400 MHz, DMSO-d6): δ ppm 8.06 (br d, J=7.53 Hz, 1H), 7.80 (br d, J=9.29 Hz, 3H), 7.14 (s, 2H), 5.21 (d, J=2.76 Hz, 3H), 4.97 (dd, J=11.17, 2.89 Hz, 3H), 4.55 (d, J=8.53 Hz, 3H), 4.14 (br s, 4H), 3.98-4.08 (m, 11H), 3.83-3.93 (m, 3H), 3.73-3.82 (m, 9H), 3.64-3.72 (m, 4H), 3.46-3.63 (m, 24H), 2.76 (t, J=5.90 Hz, 2H), 2.10 (s, 9H), 1.99 (s, 9H), 1.89 (s, 9H), 1.76 (s, 9H), 1.41 (br s, 4H), 1.14 (br d, J=6.53 Hz, 12H). ³¹P NMR: 8 ppm 144.77.

### Example 14

### Preparation of Compound 8C

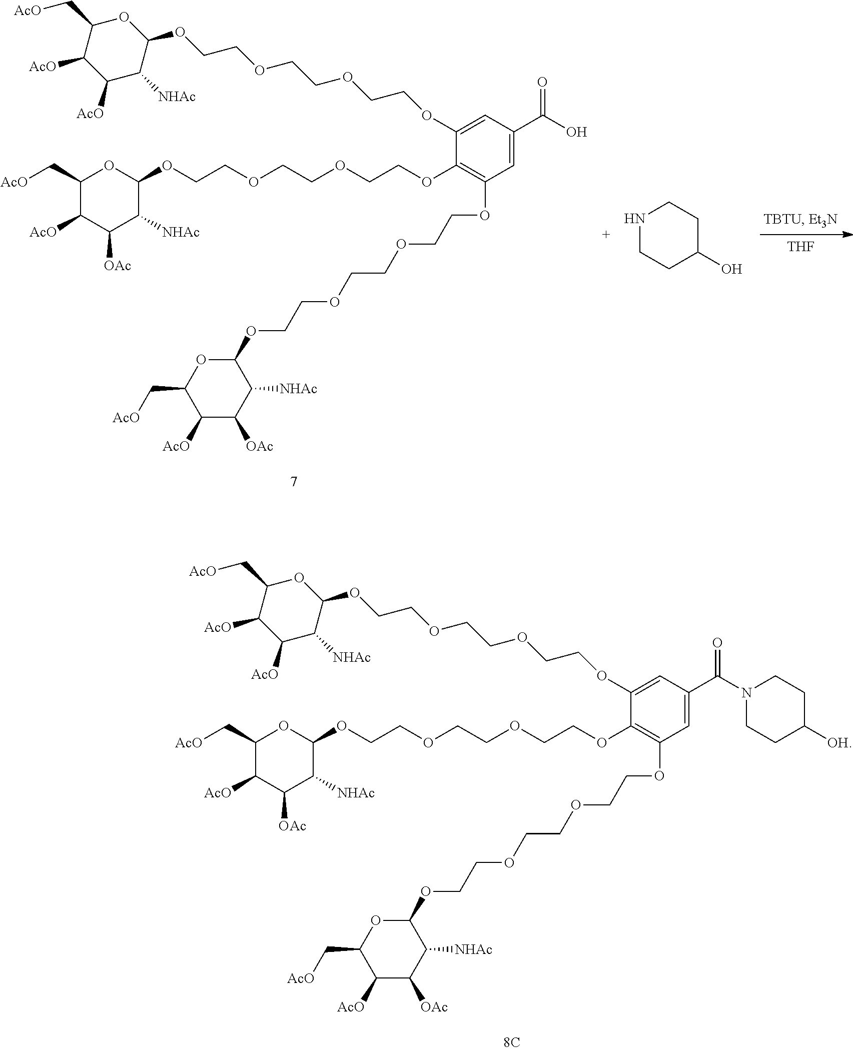

To a solution of Compound 7 (500 mg, 322 μmol) in THF (5.00 mL) was added Et₃N (65.1 mg, 643 μmol, 89.5 μL). Then TBTU (103 mg, 322 μmol) and piperidin-4-ol (32.5 mg, 322 μmol) were added to the mixture. The mixture was stirred at 20° C. for 1 hr under N₂ atmosphere. LC-MS showed Compound 7 was consumed completely and one main peak with desired m/z (Calculated MW: 1637.63, observed m/z: 819.5 [M/2+H]⁺, 1637.6 [M+H]⁺) was detected. The mixture was dissolved in DCM (100 mL), washed with HCl (1 N, 2*50 mL), organic phase was washed with saturated solution of NaHCO₃ (2*50 mL) and water (3*50 mL), then concentrated under reduced pressure to give a residue. The residue was purified by prep-HPLC (A: 0.075% TFA in H₂O, B: ACN) to give Compound 8C (390 mg, 238 μmol, 74.0% yield) as a white solid.

### Example 15

### Preparation of Ligand C

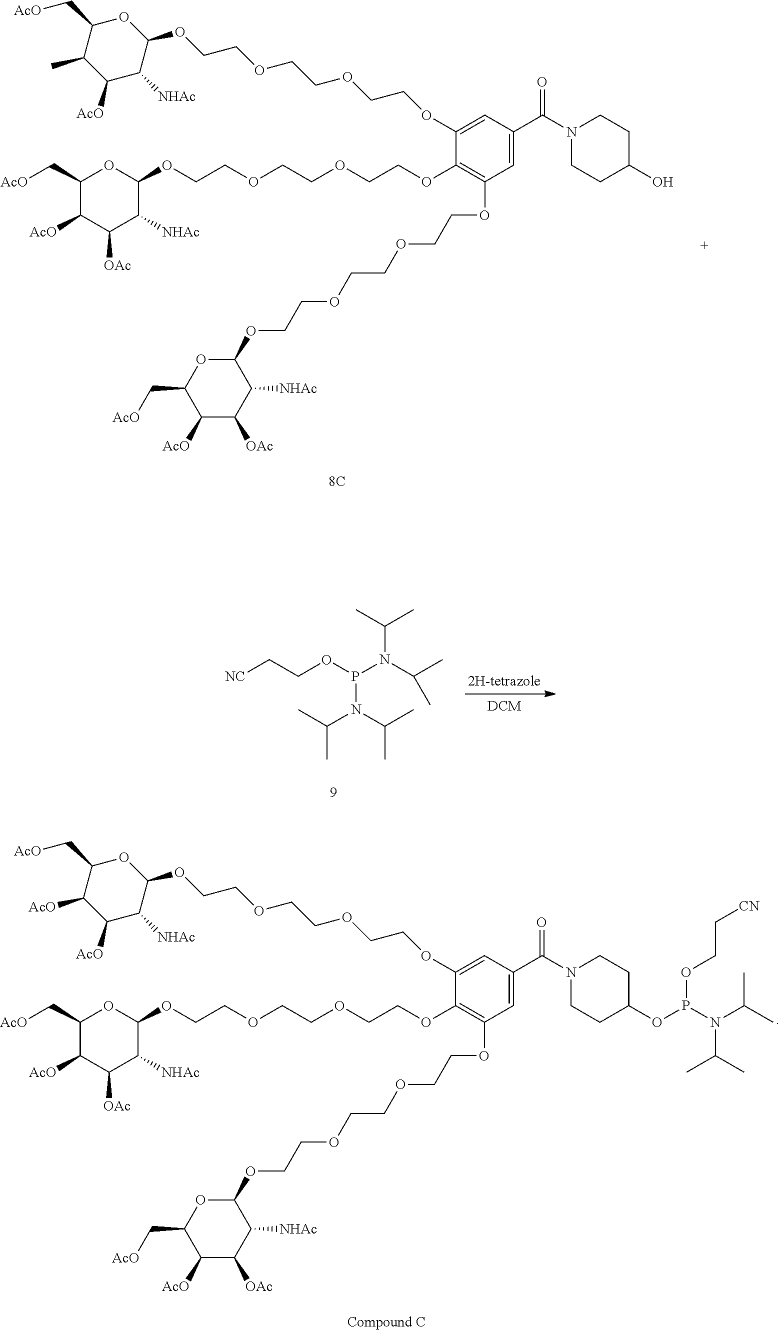

Reaction preparation: Compound 8C was dried 5 times with anhydrous MeCN (azeotropic distillation). MeCN and DCM were dried with spherical 4 Å molecular sieve overnight.

To a solution of Compound 8C (390 mg, 238 μmol) in DCM (4.00 mL) at 0° C. was added compound 9 (144 mg, 476 μmol, 151 μL) and 2H-tetrazole (0.45 M, 582 μL) dropwise under N₂ atmosphere. Then the mixture was stirred at 0-15° C. for 1 hr. TLC (dichloromethane:methyl alcohol=10:1, R_{f}=0.51) indicated Compound 8C was consumed completely and one new spot formed. The mixture was cooled to −20˜-10° C., then poured into sat. NaHCO₃ (8 mL) slowly at 0-5° C., washed with DCM (10 mL*2), the aqueous was extracted with DCM (15 mL) after separated, then the organic layer was washed with brine (15 mL), dried over Na₂SO₄, filtered and concentrated while keeping temperature below 20° C. The residue was dissolved in DCM (2 mL), added dropwise to a stirred 20 mL MTBE (−10° C.) at room temperature, stirred and filtered, washed with MTBE (10 mL*3), dried in high vacuum. This purification procedure was repeated two more times to afford Ligand C (270 mg, 147 μmol, 61.7% yield) as a white solid. ¹H NMR: (400 MHz, DMSO-d6): δ ppm 7.79 (br d, J=9.03 Hz, 3H), 6.66 (s, 2H), 5.21 (d, J=3.26 Hz, 3H), 4.97 (dd, J=11.17, 3.39 Hz, 3H), 4.53-4.58 (m, 3H), 4.10 (br d, J=4.77 Hz, 5H), 3.97-4.07 (m, 12H), 3.82-3.93 (m, 4H), 3.71-3.80 (m, 9H), 3.65-3.70 (m, 3H), 3.54-3.62 (m, 12H), 3.52 (dt, J=5.27, 2.89 Hz, 12H), 2.76 (t, J=5.77 Hz, 2H), 2.10 (s, 9H), 1.99 (s, 9H), 1.89 (s, 9H), 1.75-1.79 (m, 9H), 1.57 (br s, 2H), 1.09-1.17 (m, 14H). ³¹P NMR: δ ppm 145.39.

### Example 16

### Preparation of Compound 8D

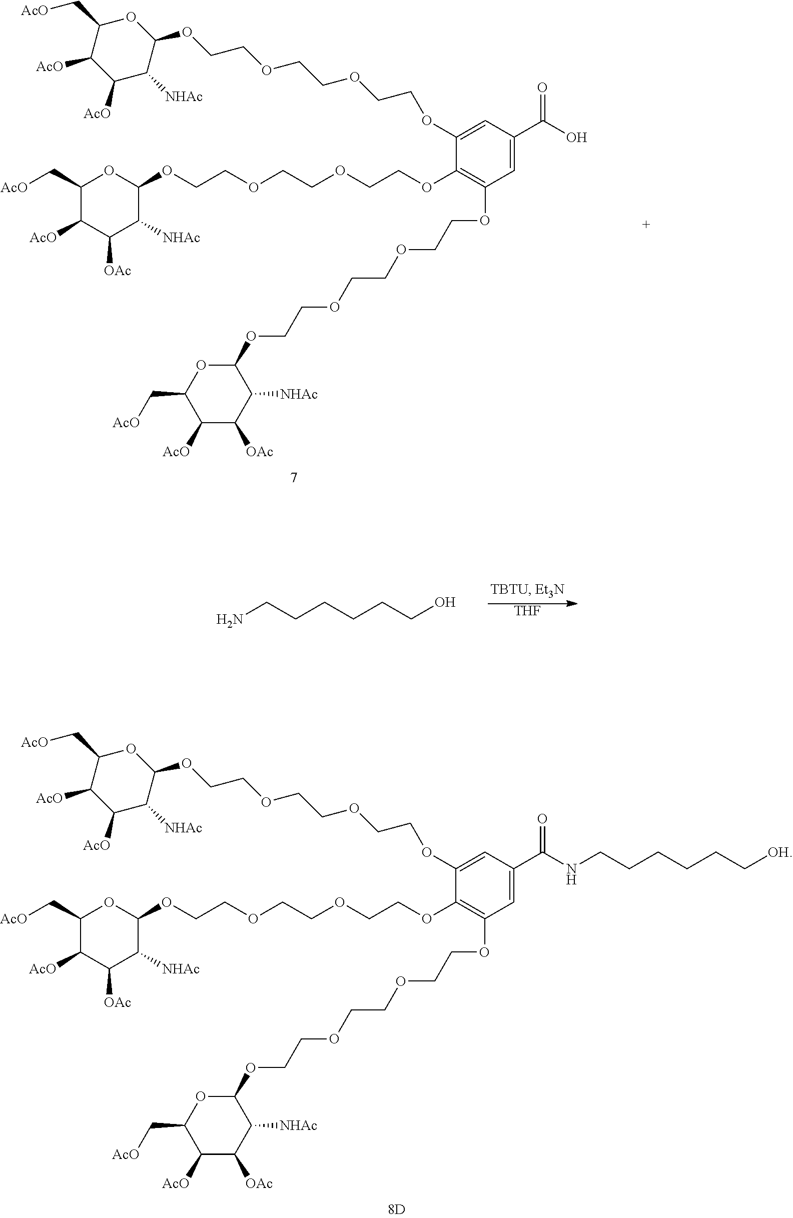

To a solution of Compound 7 (500 mg, 322 μmol) in THF (5.00 mL) was added Et₃N (65.1 mg, 643 μmol, 89.5 μL), then TBTU (103 mg, 322 μmol) and 6-aminohexan-1-ol (37.7 mg, 322 μmol) were added to the mixture. The mixture was stirred at 20° C. for 1 hr under N₂ atmosphere. LC-MS showed Compound 7 was consumed completely and one main peak with desired m/z (Calculated MW: 1653.67, observed m/z: 827.4 [M/2+H]⁺, 1654.5 [M+H]⁺) was detected. The mixture was dissolved in DCM (100 mL), washed with HCl (1 N, 2*50 mL), organic phase was washed with saturated solution of NaHCO₃ (2*50 mL) and water (3*50 mL), then concentrated under reduced pressure to give a residue. The residue was purified by prep-HPLC (A: 0.075% TFA in H₂O, B: ACN) to give Compound 8D (420 mg, 254 μmol, 79.0% yield) as a white solid.

### Example 17

### Preparation of Ligand D

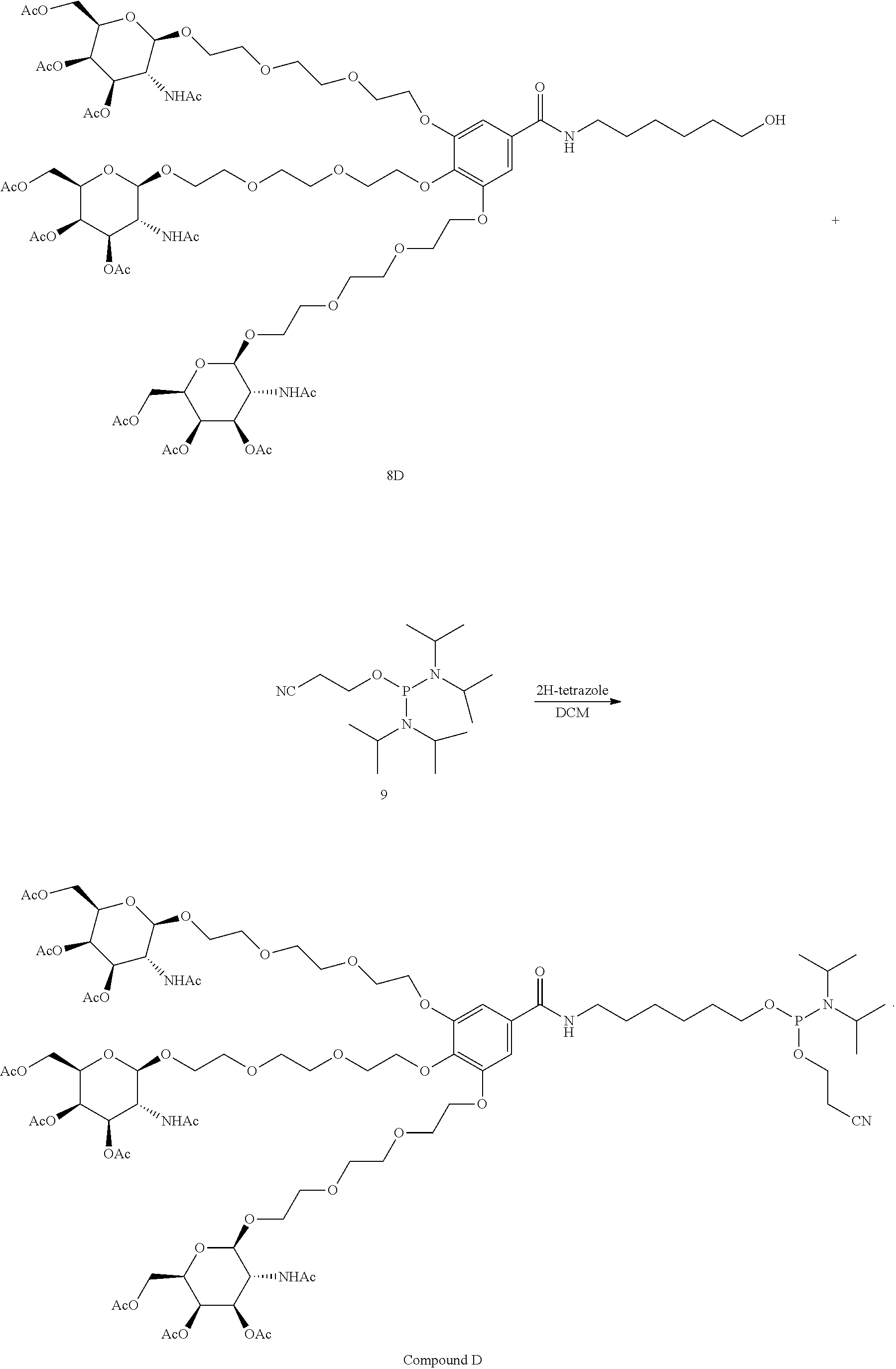

Reaction preparation: Compound 8D was dried 5 times with anhydrous MeCN (azeotropic distillation). MeCN and DCM were dried with spherical 4 Å molecular sieve overnight.

To a solution of Compound 8D (420 mg, 254 μmol) in DCM (4.00 mL) at 0° C. was added Compound 9 (153 mg, 508 μmol, 161 μL) and 2H-tetrazole (0.45 M, 621 μL) dropwise under N₂ atmosphere. Then the mixture was stirred at 0-15° C. for 1 hr. TLC (dichloromethane:methyl alcohol=10:1, R_{f}=0.51) indicated Compound 8D was consumed completely and one new spot formed. The mixture was cooled to −20˜-10° C., then poured into sat. NaHCO₃ (8 mL) slowly at 0-5° C., washed with DCM (10 mL*2), the aqueous was extracted with DCM (15 mL) after separated, then the organic layer was washed with brine (15 mL), dried over Na₂SO₄, filtered and concentrated while keeping temperature below 20° C. The residue was dissolved in DCM (2 mL), added dropwise to a stirred 20 mL MTBE (−10° C.) at room temperature, stirred and filtered, washed with MTBE (10 mL*3), dried in high vacuum. This purification procedure was repeated two more times to afford Ligand D (270 mg, 146 μmol, 57.3% yield) as a white solid. ¹H NMR (400 MHz, DMSO-d6): δ ppm 8.34 (br s, 1H), 7.80 (d, J=9.29 Hz, 3H), 7.16 (s, 2H), 5.21 (d, J=3.51 Hz, 3H), 4.97 (dd, J=11.17, 3.39 Hz, 3H), 4.55 (d, J=8.53 Hz, 3H), 4.13 (br d, J=5.02 Hz, 4H), 4.00-4.07 (m, 11H), 3.83-3.93 (m, 4H), 3.74-3.82 (m, 8H), 3.66 (br t, J=4.52 Hz, 3H), 3.58-3.63 (m, 1H), 3.59 (br d, J=4.77 Hz, 7H), 3.46-3.57 (m, 18H), 2.75 (t, J=5.90 Hz, 2H), 2.10 (s, 9H), 1.99 (s, 9H), 1.89 (s, 9H), 1.87-1.90 (m, 1H), 1.76 (s, 9H), 1.46-1.59 (m, 4H), 1.34 (br s, 4H), 1.09-1.16 (m, 12H). ³¹P NMR: 8 ppm 146.28.

### Example 18

### Preparation of Compound 8E

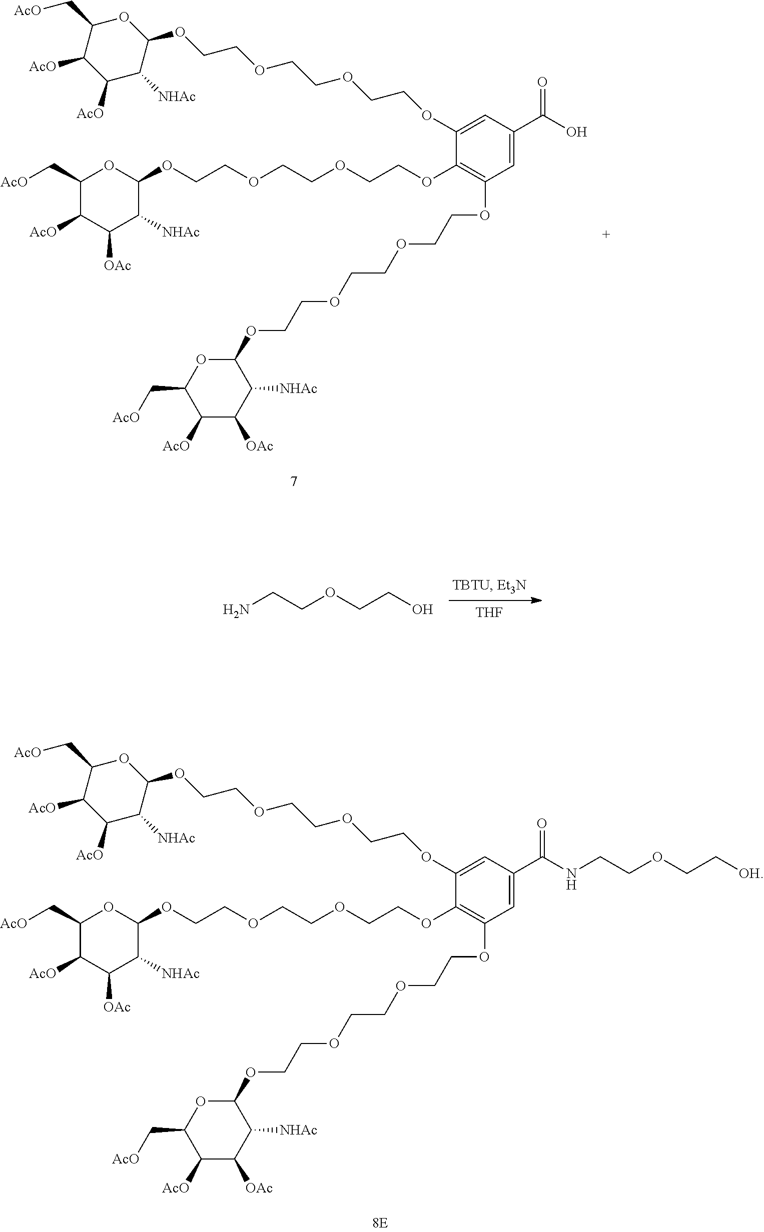

To a solution of Compound 7 (500 mg, 322 μmol) in THF (5.00 mL) was added Et₃N (65.1 mg, 643 μmol, 89.5 μL). Then TBTU (103 mg, 322 μmol) and 2-(2-aminoethoxy)ethanol (33.8 mg, 322 μmol, 32.2 μL) were added to the mixture. The mixture was stirred at 20° C. for 1 hr under N₂ atmosphere. LC-MS showed Compound 7 was consumed completely and one main peak with desired m/z (Calculated MW: 1641.62, observed m/z: 821.4 [M/2+H]⁺, 1641.5 [M+H]⁺) was detected. The mixture was dissolved in DCM (100 mL), washed with HCl (1 N, 2*50 mL), organic phase was washed with saturated solution of NaHCO₃ (2*50 mL) and water (3*50 mL), then concentrated under reduced pressure to give a residue. The residue was purified by prep-HPLC (A: 0.075% TFA in H₂O, B: ACN) to give Compound 8E (414 mg, 252.19 μmol, 78.41% yield) as a white solid.

### Example 19

### Preparation of Ligand E

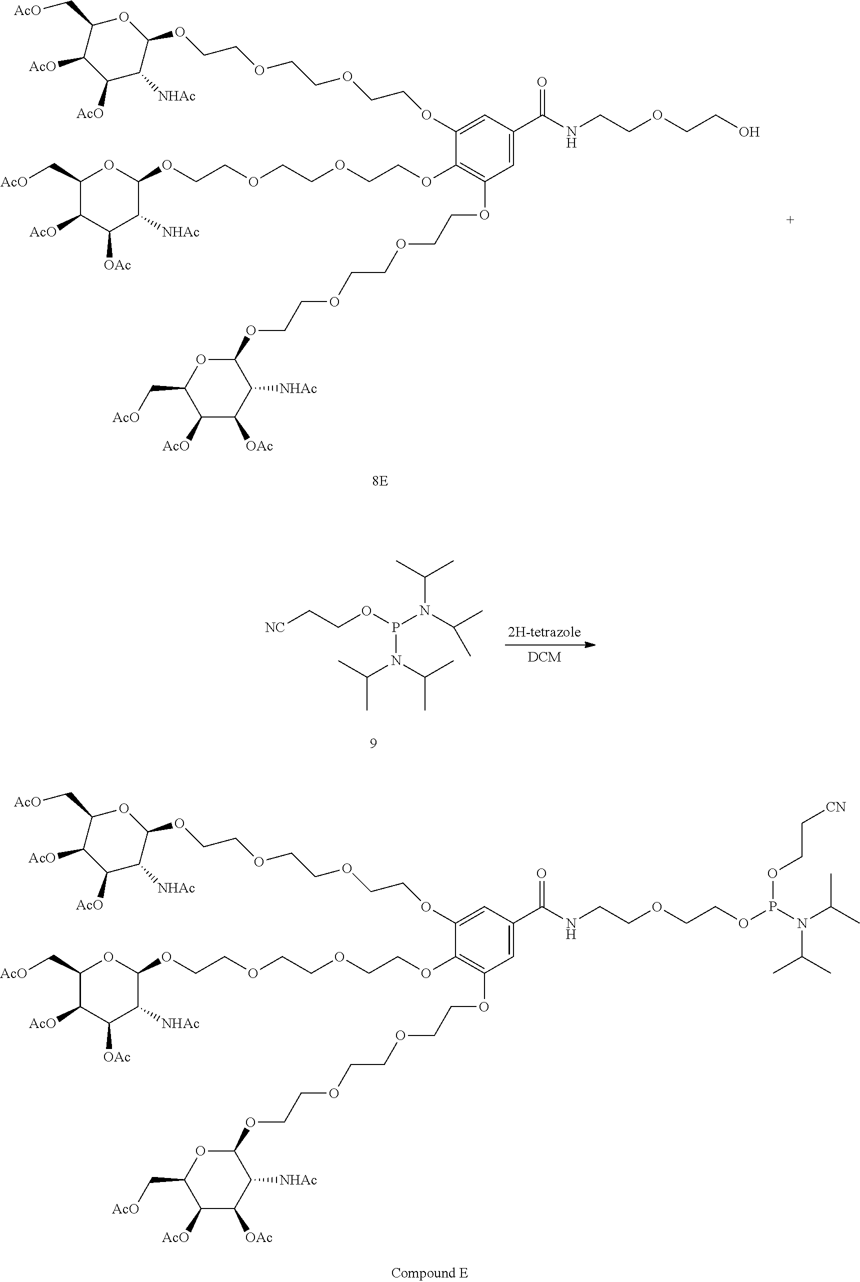

Reaction preparation: Compound 8E was dried 5 times with anhydrous MeCN (azeotropic distillation). MeCN and DCM were dried with spherical 4 Å molecular sieve overnight.

To a solution of Compound 8E (414 mg, 252 μmol) in DCM (4.00 mL) at 0° C. was added Compound 9 (152 mg, 504 μmol, 160 μL) and 2H-tetrazole (0.45 M, 616 μL) dropwise under N₂ atmosphere. Then the mixture was stirred at 0-15° C. for 1 hr. TLC (dichloromethane:methyl alcohol=10:1, R_{f}=0.52) indicated Compound 8E was consumed completely and one new spot formed. The mixture was cooled to −20˜-10° C., then poured into sat. NaHCO₃ (8 mL) slowly at 0-5° C., washed with DCM (10 mL*2), the aqueous was extracted with DCM (15 mL) after separated, then the organic layer was washed with brine (15 mL), dried over Na₂SO₄, filtered and concentrated <20° C. The residue was dissolved in DCM (2 mL), added dropwise to a stirred 20 mL MTBE (−10° C.) at room temperature, stirred and filtered, washed with MTBE (10 mL*3), dried in high vacuum. This purification procedure was repeated two more times to afford Ligand E (213 mg, 116 μmol, 45.9% yield) as a white solid. ¹H NMR: (400 MHz, DMSO-d6): δ ppm 8.45 (br t, J=5.50 Hz, 1H), 7.80 (d, J=9.13 Hz, 3H), 7.18 (s, 2H), 5.21 (d, J=3.13 Hz, 3H), 4.97 (dd, J=11.26, 3.25 Hz, 3H), 4.55 (d, J=8.50 Hz, 3H), 4.10-4.18 (m, 4H), 3.96-4.09 (m, 11H), 3.83-3.93 (m, 3H), 3.64-3.82 (m, 13H), 3.46-3.62 (m, 28H), 2.73 (t, J=5.69 Hz, 2H), 2.10 (s, 9H), 1.99 (s, 9H), 1.89 (s, 9H), 1.76 (s, 9H), 1.11 (t, J=6.00 Hz, 12H). ³¹P NMR: δ ppm 147.28.

### Example 20

### Preparation of Compound 8F

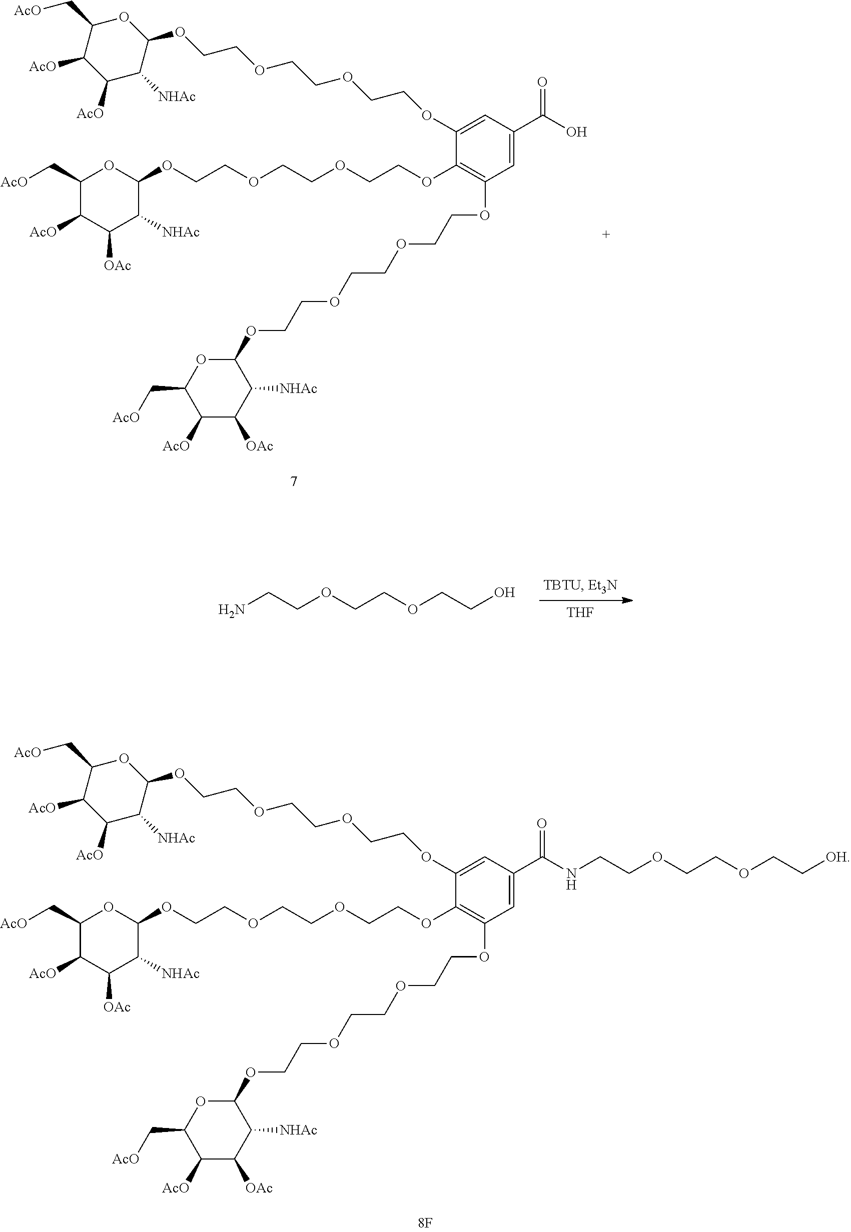

To a solution of Compound 7 (700 mg, 450 μmol) in THF (7.00 mL) was added Et₃N (91.1 mg, 901 μmol, 125 μL). Then TBTU (145 mg, 450 μmol) and 2-[2-(2-aminoethoxy)ethoxy]ethanol (67.2 mg, 450 μmol) was added to the mixture. The mixture was stirred at 20° C. for 1 hr. LC-MS showed Compound 7 was consumed completely and one main peak with desired m/z (Calculated MW: 1685.67, observed m/z: 843.5 [M/2+H]⁺, 1685.5 [M+H]⁺) was detected. The mixture was dissolved in DCM (50 mL), washed with HCl (1 N, 2*25 mL), organic phase was washed with saturated solution of NaHCO₃ (2*30 mL) and water (3*50 mL), then concentrated under reduced pressure to give a residue. The residue was purified by prep-HPLC (A: 0.075% TFA in H₂O, B: ACN) to give Compound 8F (640 mg, 380 μmol, 84.3% yield) as a white solid.

### Example 21

### Preparation of Ligand F

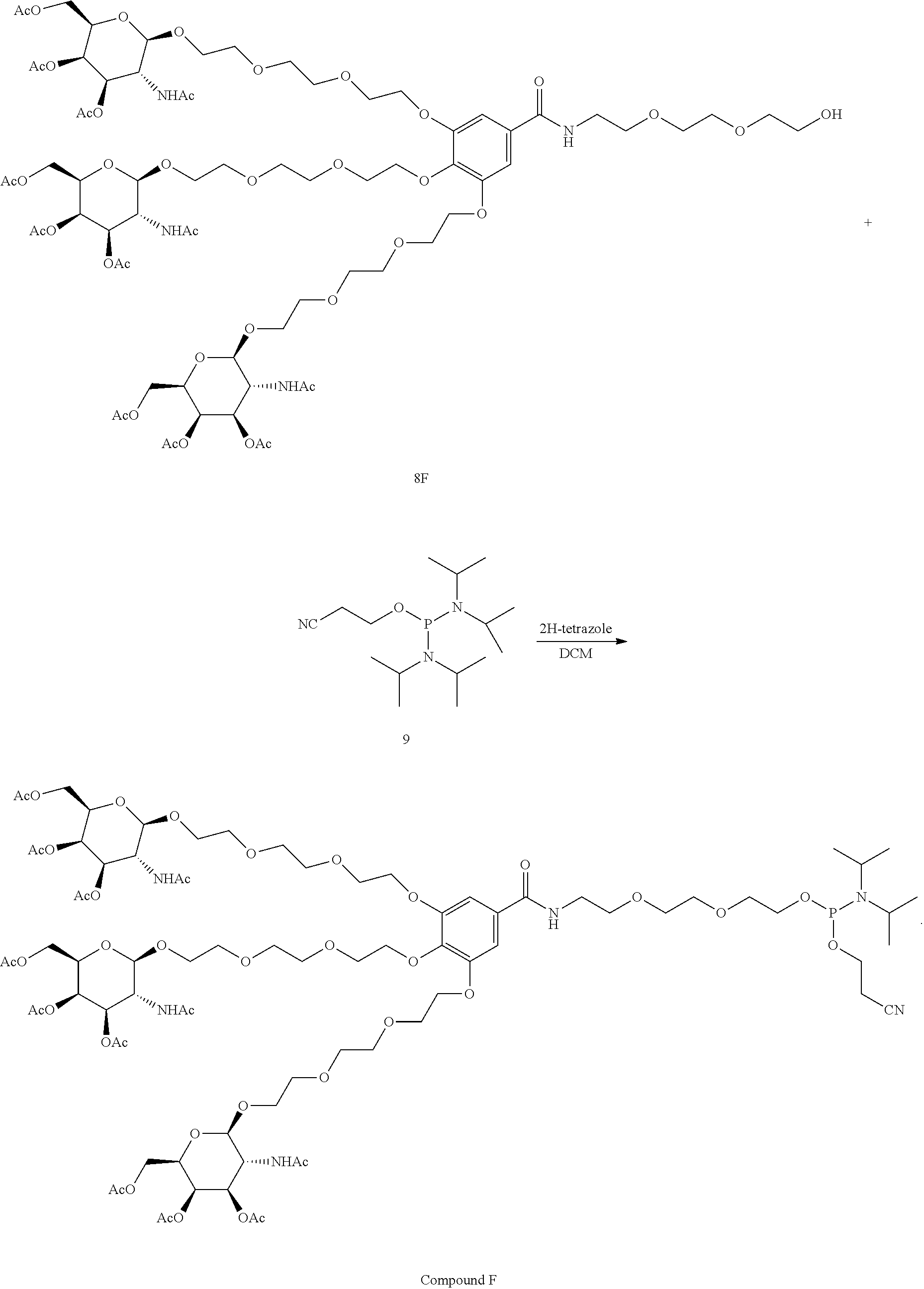

Reaction preparation: Compound 8F was dried 5 times with anhydrous MeCN (azeotropic distillation). MeCN and DCM were dried with spherical 4 Å molecular sieve overnight.

To a solution of Compound 8F (540 mg, 320 μmol) in DCM (6.00 mL) at 0° C. was added Compound 9 (193 mg, 641 μmol, 203 μL), then 2H-tetrazole (0.45 M, 783 μL) was added dropwise to the reaction mixture. The mixture was stirred at 10-15 0-15° C. under N₂ atmosphere for 1 hr. TLC (dichloromethane:methyl alcohol=10:1, R_{f}=0.53) indicated Compound 8F was consumed completely and one new spot formed. The mixture was cooled to −20˜−10° C., then poured into sat. NaHCO₃ (8 mL) slowly at 0-5° C., washed with DCM (10 mL*2), the aqueous was extracted with DCM (15 mL) after separated, then the organic layer was washed with brine (15 mL), dried over Na₂SO₄, filtered and concentrated while keeping temperature below 20° C. The residue was dissolved in DCM (2 mL), added dropwise to a stirred 20 mL MTBE (−10° C.) at room temperature, stirred and filtered, washed with MTBE (10 mL*3), dried in high vacuum. This purification procedure was repeated two more times to afford Ligand F (412 mg, 218 μmol, 68.2% yield) as a white solid. ¹H NMR (400 MHz, DMSO-d6): δ ppm 8.46 (br t, J=5.44 Hz, 1H), 7.80 (d, J=9.26 Hz, 3H), 7.18 (s, 2H), 5.21 (d, J=3.38 Hz, 3H), 4.97 (dd, J=11.26, 3.38 Hz, 3H), 4.55 (d, J=8.50 Hz, 3H), 4.13 (br t, J=4.38 Hz, 4H), 3.98-4.07 (m, 11H), 3.83-3.92 (m, 3H), 3.73-3.82 (m, 8H), 3.71 (td, J=4.19, 2.38 Hz, 2H), 3.64-3.69 (m, 3H), 3.46-3.62 (m, 33H), 2.75 (t, J=5.94 Hz, 2H), 2.10 (s, 9H), 1.99 (s, 9H), 1.89 (s, 9H), 1.76 (s, 9H), 1.19 (br d, J=6.38 Hz, 2H), 1.12 (dd, J=6.69, 4.19 Hz, 10H). ³¹P NMR: δ ppm 147.35.

### Example 22

### Preparation of Compound 5′

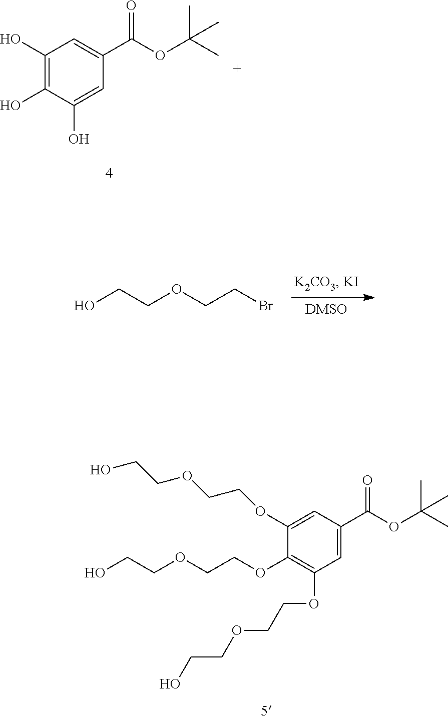 [SMILES: CC(C)(C)OC(=O)c1cc(O)c(O)c(O)c1 ; OCCOCCBr ; CC(C)(C)OC(=O)c1cc(OCCOCCO)c(OCCOCCO)c(OCCOCCO)c1]

To a solution of Compound 4 (2.00 g, 8.84 mmol) in DMSO (40.0 mL) was added K₂CO₃ (4.89 g, 35.4 mmol) and KI (440 mg, 2.65 mmol) and stirred until temperature warm to 70° C. Then 2-(2-bromoethoxy)ethanol (5.98 g, 35.4 mmol) was added to the mixture. The mixture was stirred at 70° C. for 4 hr. LC-MS showed Compound 4 was consumed completely and one main peak with desired m/z (Calculated MW: 490.54, observed m/z: 491.2 [M+H]⁺) was detected. The reaction mixture was purified by prep-HPLC (neutral condition) to give Compound 5′ (3.40 g, 6.93 mmol, 78.4% yield) as a pale brown oil. ¹H NMR (400 MHz, CHLOROFORM-d): 8 ppm 7.29 (s, 2H), 3.86-3.91 (m, 4H), 3.80-3.85 (m, 2H), 3.71-3.78 (m, 6H), 3.63-3.69 (m, 6H), 1.67 (s, 9H).

### Example 23

### Preparation of Compound 6′

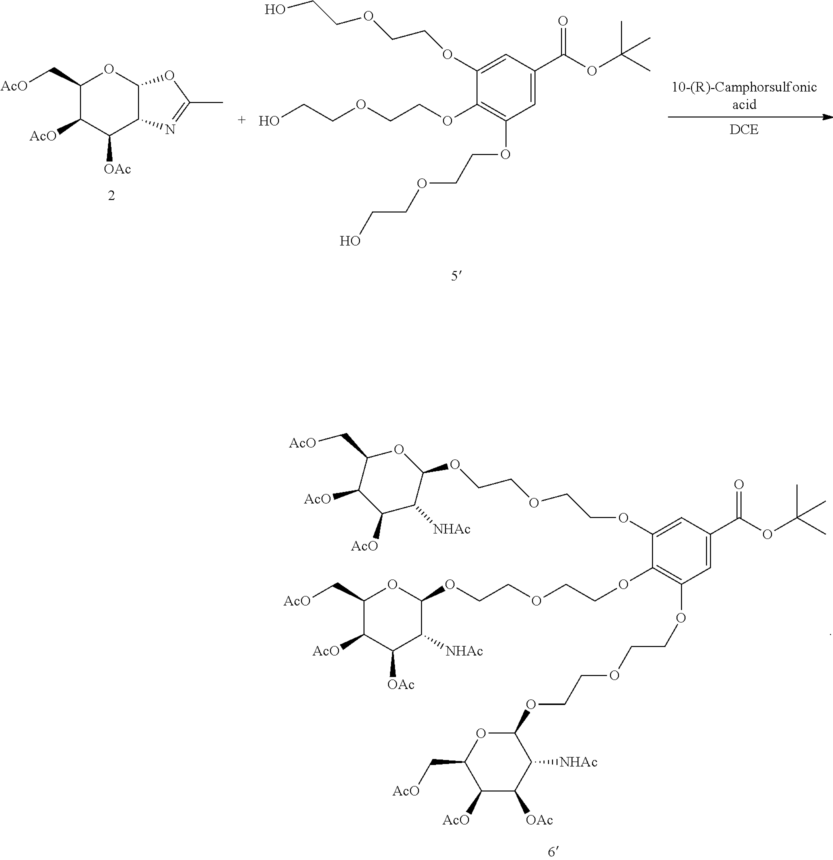 [SMILES: CC(=O)OC[C@H]1O[C@@H]2OC(C)=N[C@@H]2[C@@H](OC(C)=O)[C@H]1OC(C)=O ; CC(C)(C)OC(=O)c1cc(OCCOCCO)c(OCCOCCO)c(OCCOCCO)c1 ; CC(=O)N[C@H]1[C@H](OCCOCCOc2cc(C(=O)OC(C)(C)C)cc(OCCOCCO[C@@H]3O[C@H](COC(C)=O)[C@H](OC(C)=O)[C@H](OC(C)=O)[C@H]3NC(C)=O)c2OCCOCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

Compound 2 (12.1 g, 36.7 mmol) in anhydrous DCE (120 mL) was stirred with 4 Å molecular sieves for 5 min at 20° C. Compound 5′ (3.00 g, 6.12 mmol) was added and stirring was continued for 30 min. Then [(1R,4S)-7,7-dimethyl-2-oxo-norbornan-1-yl]methanesulfonic acid (5.11 g, 22.0 mmol) was added dropwise over 10 min under N₂ atmosphere. The mixture was stirred at 50° C. for 2 hr. TLC (dichloromethane/methanol=10:1, R_{f}=0.36 [bromocresol green]) indicated Compound 5′ was consumed remaining little and one new spot formed. The reaction was clean according to TLC. The residue was extracted with NaHCO₃ (300 mL) and DCM (300 mL*3), The combined organic layers were washed with saturated NaCl solution, dried over, filtered and concentrated under reduced pressure to give a residue. The residue was purified by column chromatography (SiO₂, dichloromethane:(dichloromethane/methanol=10:1)=0:100) to give Compound 6′ (8.80 g, 5.95 mmol, 97.3% yield) as a white solid. ¹H NMR (400 MHz, CHLOROFORM-d): 8 ppm 7.26 (s, 2H), 7.24-7.26 (m, 1H), 6.82 (d, J=9.54 Hz, 1H), 6.69 (d, J=8.78 Hz, 2H), 5.33-5.37 (m, 3H), 5.22 (dd, J=11.04, 3.26 Hz, 2H), 5.12 (dd, J=11.29, 3.26 Hz, 1H), 4.83 (d, J=8.53 Hz, 1H), 4.77 (d, J=8.53 Hz, 2H), 4.07-4.26 (m, 15H), 3.92-4.03 (m, 6H), 3.78-3.91 (m, 7H), 3.67-3.77 (m, 8H), 2.14-2.18 (m, 9H), 2.03-2.06 (m, 9H), 1.96-2.00 (m, 9H), 1.87-1.95 (m, 9H), 1.59 (s, 9H).

### Example 24

### Preparation of Compound 7′

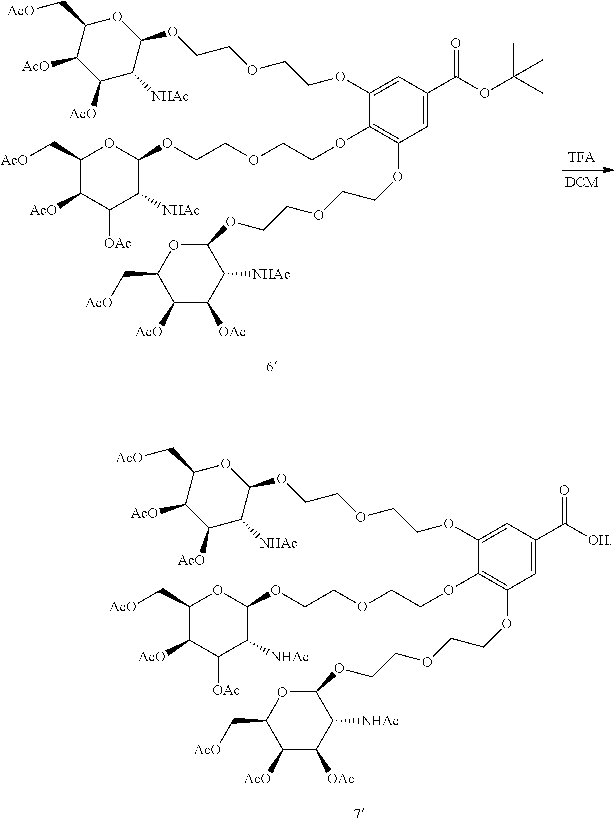 [SMILES: CC(=O)N[C@H]1[C@H](OCCOCCOc2cc(C(=O)OC(C)(C)C)cc(OCCOCCO[C@@H]3O[C@H](COC(C)=O)[C@H](OC(C)=O)[C@H](OC(C)=O)[C@H]3NC(C)=O)c2OCCOCCO[C@@H]2O[C@H](COC(C)=O)[C@H](OC(C)=O)C(OC(C)=O)[C@H]2NC(C)=O)O[C@H](COC(C)=O)[C@H](OC(C)=O)[C@@H]1OC(C)=O ; CC(=O)N[C@H]1[C@H](OCCOCCOc2cc(C(=O)O)cc(OCCOCCO[C@@H]3O[C@H](COC(C)=O)[C@H](OC(C)=O)[C@H](OC(C)=O)[C@H]3NC(C)=O)c2OCCOCCO[C@@H]2O[C@H](COC(C)=O)[C@H](OC(C)=O)C(OC(C)=O)[C@H]2NC(C)=O)O[C@H](COC(C)=O)[C@H](OC(C)=O)[C@@H]1OC(C)=O]

To a solution of Compound 6′ (8.60 g, 5.82 mmol) in DCM (43.0 mL) was added TFA (66.2 g, 581 mmol, 43.0 mL) under 0° C. The mixture was stirred at 20 0-20° C. for 1 hr. LC-MS showed Compound 6′ was consumed completely and one main peak with desired m/z (Calculated MW: 1422.34, observed m/z: 711.8 [M/2+H]⁺, 1422.4 [M+H]⁺) was detected. The reaction mixture was concentrated under reduced pressure to give a residue. The residue was purified by prep-HPLC (A: 0.075% TFA in H₂O, B: ACN) to give Compound 7′ (4.60 g, 3.23 mmol, 55.6% yield) as a white solid. ¹H NMR (400 MHz, DMSO-d6): δ ppm 7.78-7.86 (m, 2H), 7.78-7.86 (m, 1H), 7.22 (s, 2H), 5.21 (d, J=3.26 Hz, 3H), 4.94-5.01 (m, 3H), 4.53-4.59 (m, 3H), 4.07-4.16 (m, 6H), 4.02 (s, 9H), 3.84-3.93 (m, 4H), 3.72-3.83 (m, 7H), 3.65-3.69 (m, 2H), 3.56-3.65 (m, 10H), 2.10 (s, 9H), 1.96-2.02 (m, 1H), 1.99 (s, 8H), 1.86-1.91 (m, 9H), 1.76 (s, 9H).

### Example 25

### Preparation of Compound 8G

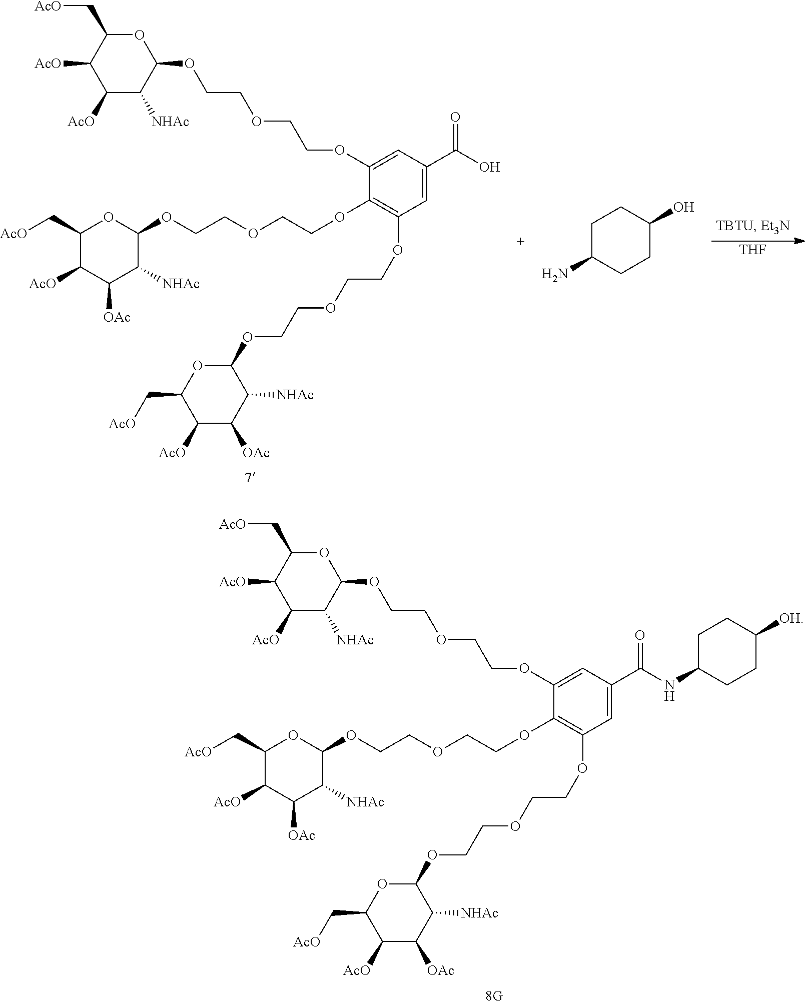

To a solution of Compound 7′ (500 mg, 352 μmol) in THF (5.00 mL) was added Et₃N (71.1 mg, 703 μmol, 97.9 μL). Then TBTU (113 mg, 352 μmol) and 4-aminocyclohexanol (40.5 mg, 352 μmol) were added to the mixture. The mixture was stirred at 20° C. for 1 hr under N₂ atmosphere. LC-MS showed Compound 7′ was consumed completely and one main peak with desired m/z (Calculated MW: 1519.50, observed m/z: 760.4 [M/2+H]⁺, 1519.4 [M+H]⁺) was detected. The mixture was dissolved in DCM (100 mL), washed with HCl (1 N, 2*50 mL), organic phase was washed with saturated solution of NaHCO₃ (2*50 mL) and water (3*50 mL), then concentrated under reduced pressure to give a residue. The residue was purified by prep-HPLC (A: 0.075% TFA in H₂O, B: ACN) to give Compound 8G (430 mg, 283 μmol, 80.5% yield) as a white solid.

### Example 26

### Preparation of Ligand G

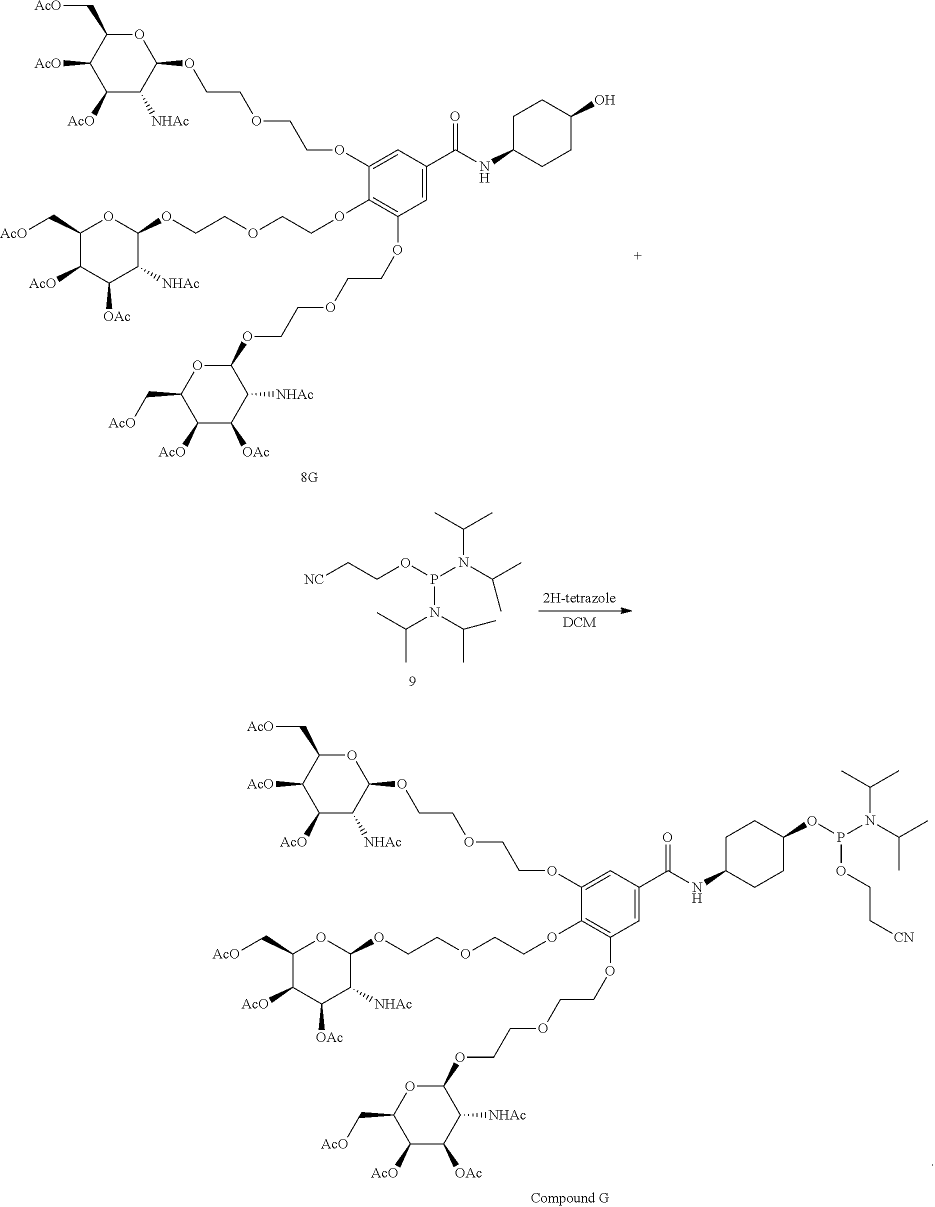

Reaction preparation: Compound 8G was dried 5 times with anhydrous MeCN (azeotropic distillation). MeCN and DCM were dried with spherical 4 Å molecular sieve overnight.

To a solution of Compound 8G (430 mg, 283 μmol) in DCM (4.00 mL) at 0° C. was added Compound 9 (171 mg, 566 μmol, 180 μL) and 2H-tetrazole (0.45 M, 692 μL) dropwise under N₂ atmosphere. Then the mixture was stirred at 0-15° C. for 1 hr. TLC (dichloromethane:methyl alcohol=10:1, R_{f}=0.52) indicated Compound 8G was consumed completely and one new spot formed. The mixture was cooled to −20˜-10° C., then poured into sat. NaHCO₃ (8 mL) slowly at 0-5° C., washed with DCM (10 mL*2), the aqueous was extracted with DCM (15 mL) after separated, then the organic layer was washed with brine (15 mL), dried over Na₂SO₄, filtered and concentrated while keeping temperature below 20° C. The residue was dissolved in DCM (2 mL), added dropwise to a stirred 20 mL MTBE (−10° C.) at room temperature, stirred and filtered, washed with MTBE (10 mL*3), dried in high vacuum. This purification procedure was repeated two more times to afford Ligand G (370 mg, 215 μmol, 76.0% yield) as a white solid. ¹H NMR (400 MHz, DMSO-d6): δ ppm 8.13-8.19 (m, 1H), 8.16 (br d, J=7.53 Hz, 1H), 7.78-7.86 (m, 3H), 7.18 (s, 2H), 5.21 (d, J=3.51 Hz, 3H), 4.98 (dd, J=11.29, 3.26 Hz, 3H), 4.53-4.60 (m, 3H), 4.13 (br t, J=4.39 Hz, 4H), 3.99-4.07 (m, 12H), 3.86-3.94 (m, 3H), 3.71-3.86 (m, 10H), 3.53-3.69 (m, 14H), 2.77 (t, J=5.90 Hz, 2H), 2.10 (s, 9H), 1.99 (s, 9H), 1.89 (s, 9H), 1.77 (s, 9H), 1.54-1.73 (m, 6H), 1.16 (d, J=6.78 Hz, 12H), 1.10 (s, 2H). ³¹P NMR: δ ppm 145.74.

### Example 27

### Preparation of Compound 8H

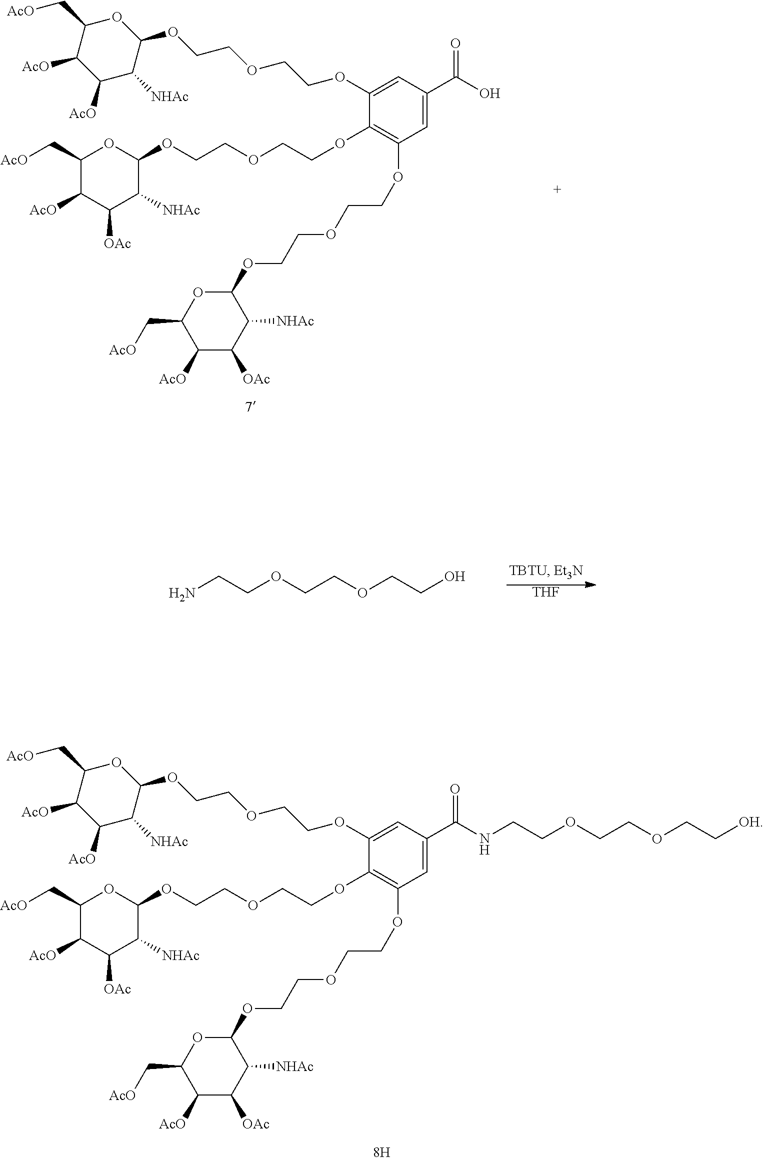 [SMILES: CC(=O)N[C@H]1[C@H](OCCOCCOc2cc(C(=O)O)cc(OCCOCCO[C@@H]3O[C@H](COC(C)=O)[C@H](OC(C)=O)[C@H](OC(C)=O)[C@H]3NC(C)=O)c2OCCOCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O ; NCCOCCOCCO ; "THF" ; CC(=O)N[C@H]1[C@H](OCCOCCOc2cc(C(=O)NCCOCCOCCO)cc(OCCOCCO[C@@H]3O[C@H](COC(C)=O)[C@H](OC(C)=O)[C@H](OC(C)=O)[C@H]3NC(C)=O)c2OCCOCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O]

To a solution of Compound 7′ (500 mg, 352 μmol) in THF (5.00 mL) was added Et₃N (71.1 mg, 703 μmol, 97.9 μL). Then TBTU (113 mg, 352 μmol) and 2-[2-(2-aminoethoxy)ethoxy]ethanol (52.4 mg, 352 μmol) were added to the mixture. The mixture was stirred at 20° C. for 1 hr under N₂ atmosphere. LC-MS showed Compound 7′ was consumed completely and one main peak with desired m/z (Calculated MW: 1553.52, observed m/z: 777.3 [M/2+H]⁺, 1554.5 [M+H]⁺) was detected. The mixture was dissolved in DCM (100 mL), washed with HCl (1 N, 2*50 mL), organic phase was washed with saturated solution of NaHCO₃ (2*50 mL) and water (3*50 mL), then concentrated under reduced pressure to give a residue. The residue was purified by prep-HPLC (A: 0.075% TFA in H₂O, B: ACN) to give Compound 8H (458 mg, 295 μmol, 83.9% yield) as a white solid.

### Example 28

### Preparation of Ligand H

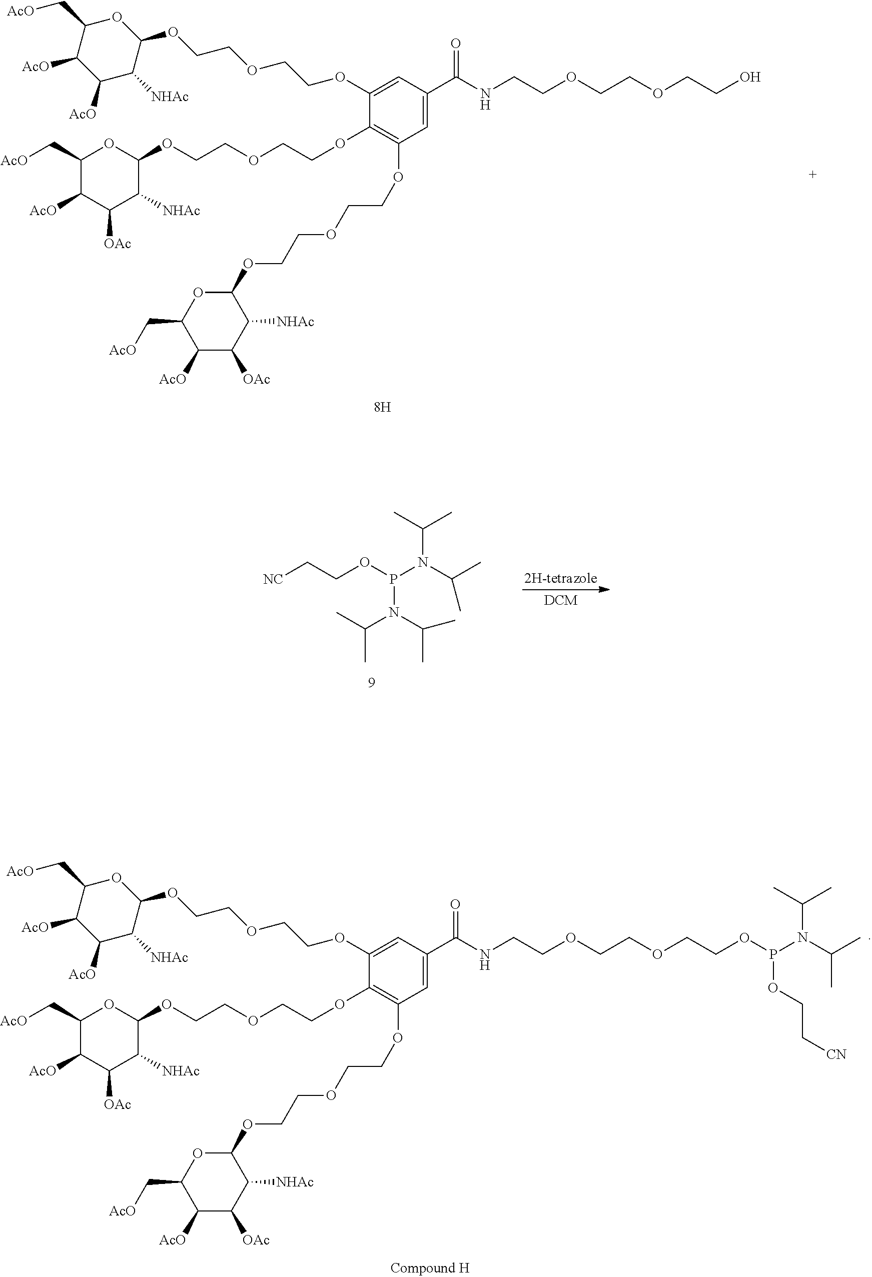

Reaction preparation: Compound 8H was dried 5 times with anhydrous MeCN (azeotropic distillation). MeCN and DCM were dried with spherical 4 Å molecular sieve overnight.

To a solution of Compound 8H (458 mg, 295 μmol) in DCM (4.70 mL) at 0° C. was added Compound 9 (178 mg, 590 μmol, 187 μL) and 2H-tetrazole (0.45 M, 721 μL) dropwise under N₂ atmosphere. Then the mixture was stirred at 0-15° C. for 1 hr. TLC (dichloromethane:methyl alcohol=10:1, R_{f}=0.52) indicated Compound 8H was consumed completely and one new spot formed. The mixture was cooled to −20˜-10° C., then poured into sat. NaHCO₃ (8 mL) slowly at 0-5° C., washed with DCM (10 mL*2), the aqueous was extracted with DCM (15 mL) after separated, then the organic layer was washed with brine (15 mL), dried over Na₂SO₄, filtered and concentrated while keeping temperature below 20° C. The residue was dissolved in DCM (2 mL), added dropwise to a stirred 20 mL MTBE (−10° C.) at room temperature, stirred and filtered, washed with MTBE (10 mL*3), dried in high vacuum. This purification procedure was repeated two more times to afford Ligand H (250 mg, 143 μmol, 48.4% yield) as a white solid. ¹H NMR (400 MHz, DMSO-d6): δ ppm 8.36 (br s, 1H), 7.77-7.88 (m, 3H), 7.17 (s, 2H), 5.21 (d, J=3.26 Hz, 3H), 4.93-5.02 (m, 3H), 4.52-4.62 (m, 3H), 4.12 (br t, J=4.52 Hz, 4H), 4.03 (s, 12H), 3.86-3.94 (m, 3H), 3.78-3.85 (m, 3H), 3.70-3.77 (m, 5H), 3.52-3.69 (m, 16H), 3.22 (br d, J=6.78 Hz, 2H), 2.75 (t, J=5.90 Hz, 2H), 2.10 (s, 9H), 1.99 (s, 9H), 1.89 (s, 9H), 1.76 (s, 9H), 1.47-1.58 (m, 4H), 1.33 (br s, 4H), 1.06-1.19 (m, 12H). ³¹P NMR: 8 ppm 147.36.

### Example 29

### Preparation of Compound 8I

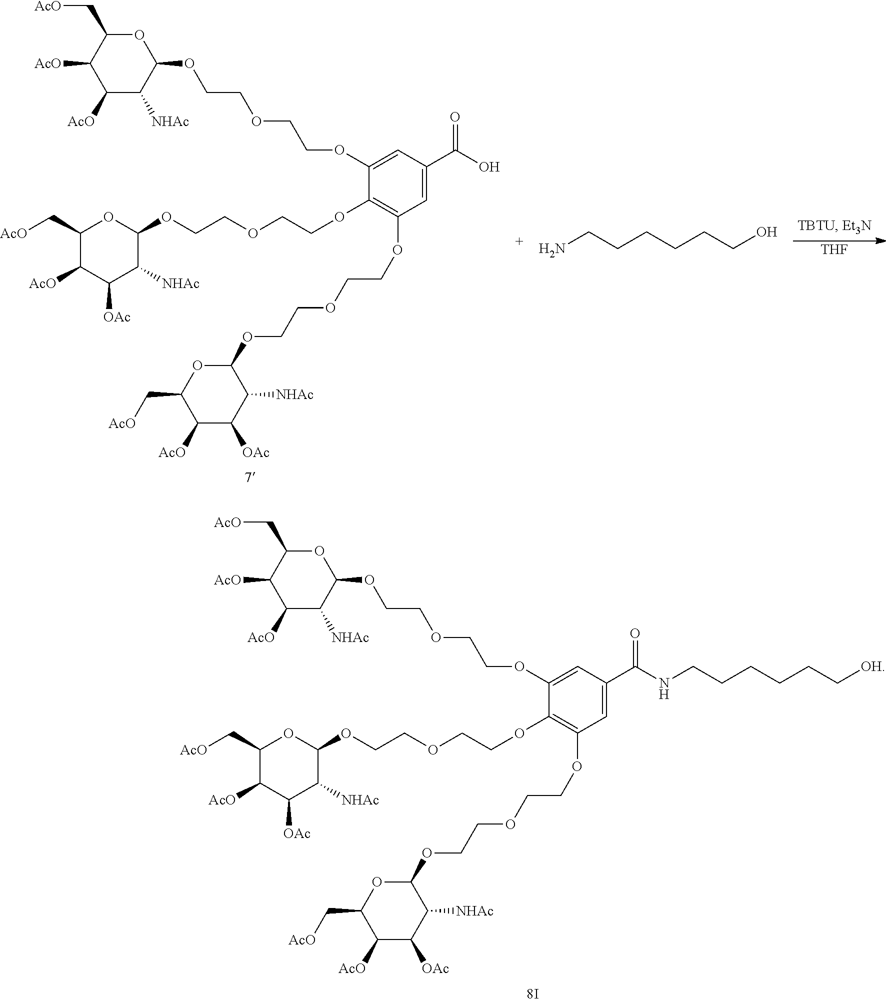 [SMILES: CC(=O)N[C@H]1[C@H](OCCOCCOc2cc(C(=O)O)cc(OCCOCCO[C@@H]3O[C@H](COC(C)=O)[C@H](OC(C)=O)[C@H](OC(C)=O)[C@H]3NC(C)=O)c2OCCOCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O ; NCCCCCCO ; CC(=O)N[C@H]1[C@H](OCCOCCOc2cc(C(=O)NCCCCCCO)cc(OCCOCCO[C@@H]3O[C@H](COC(C)=O)[C@H](OC(C)=O)[C@H](OC(C)=O)[C@H]3NC(C)=O)c2OCCOCCO[C@@H]2O[C@H](COC(C)=O)[C@H](OC(C)=O)[C@H](OC(C)=O)[C@H]2NC(C)=O)O[C@H](COC(C)=O)[C@H](OC(C)=O)[C@@H]1OC(C)=O ; "THF"]

To a solution of Compound 7′ (500 mg, 352 μmol) in THF (5.00 mL) was added Et₃N (71.1 mg, 703 μmol, 97.9 μL). Then TBTU (113 mg, 352 μmol) and 6-aminohexan-1-ol (41.2 mg, 352 μmol) were added to the mixture. The mixture was stirred at 20° C. for 1 hr under N₂ atmosphere. LC-MS showed Compound 7′ was consumed completely and one main peak with desired m/z (Calculated MW: 1521.52, observed m/z: 761.3 [M/2+H]⁺, 1522.5 [M+H]⁺) was detected. The mixture was dissolved in DCM (100 mL), washed with HCl (1 N, 2*50 mL), organic phase was washed with saturated solution of NaHCO₃ (2*50 mL) and water (3*50 mL), then concentrated under reduced pressure to give a residue. The residue was purified by prep-HPLC (A: 0.075% TFA in H₂O, B: ACN) to give Compound 8I (473 mg, 311 μmol, 88.4% yield) as a white solid.

### Example 30

### Preparation of Ligand I

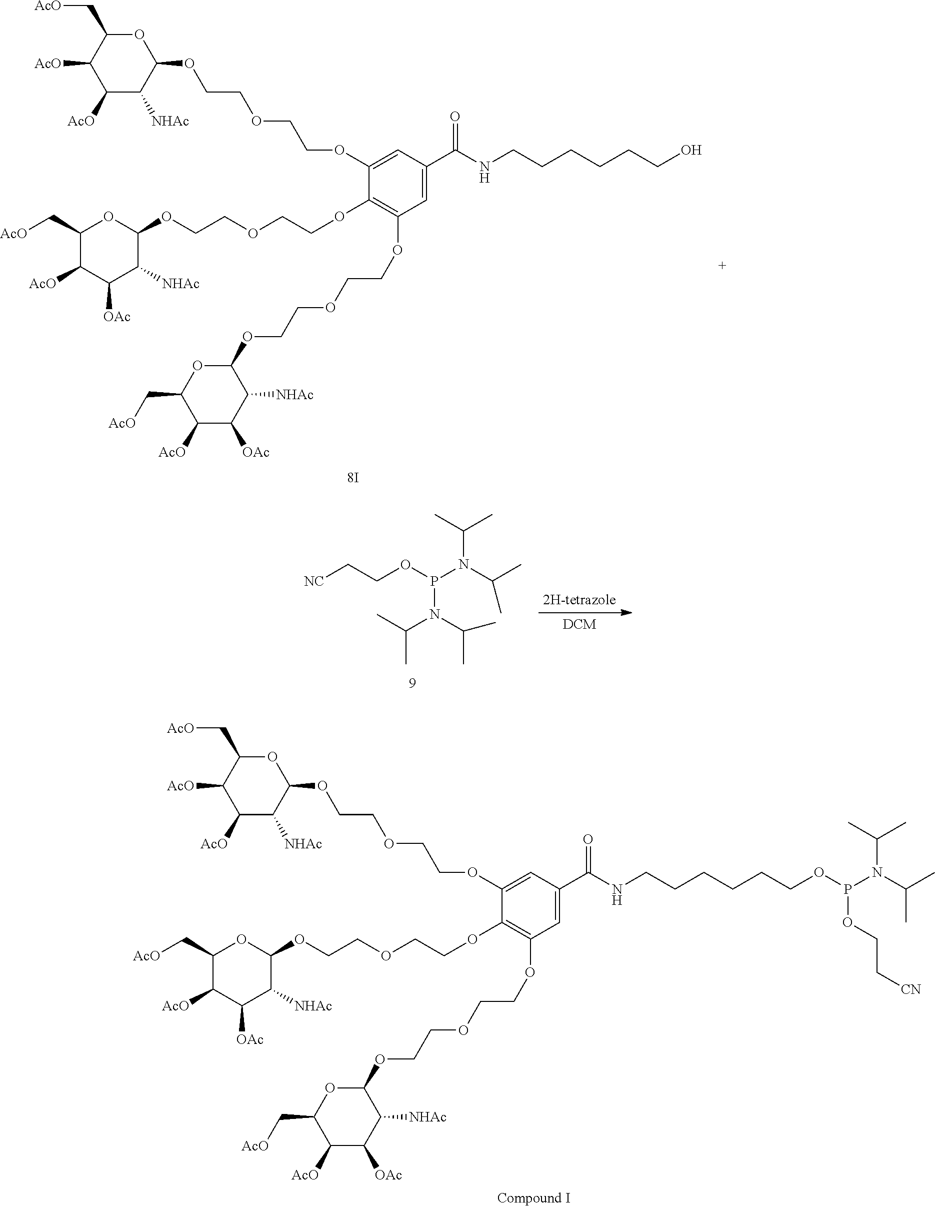

Reaction preparation: Compound 8I was dried 5 times with anhydrous MeCN (azeotropic distillation). MeCN and DCM were dried with spherical 4 Å molecular sieve overnight.

To a solution of Compound 8I (473 mg, 311 μmol) in DCM (5.00 mL) at 0° C. was added Compound 9 (187 mg, 622 μmol, 197 μL) and 2H-tetrazole (0.45 M, 691 μL) dropwise under N₂ atmosphere. Then the mixture was stirred at 0-15° C. for 1 hr. TLC (dichloromethane:methyl alcohol=10:1, R_{f}=0.53) indicated Compound 8I was consumed completely and one new spot formed. The mixture was cooled to −20˜-10° C., then poured into sat. NaHCO₃ (8 mL) slowly at 0-5° C., washed with DCM (10 mL*2), the aqueous was extracted with DCM (15 mL) after separated, then the organic layer was washed with brine (15 mL), dried over Na₂SO₄, filtered and concentrated while keeping temperature below 20° C. The residue was dissolved in DCM (2 mL), added dropwise to a stirred 20 mL MTBE (−10° C.) at room temperature, stirred and filtered, washed with MTBE (10 mL*3), dried in high vacuum. This purification procedure was repeated two more times to afford Ligand I (350 mg, 203 μmol, 65.4% yield) as a white solid. ¹H NMR (400 MHz, DMSO-d6): δ ppm 8.36 (br s, 1H), 7.77-7.88 (m, 3H), 7.17 (s, 2H), 5.21 (d, J=3.26 Hz, 3H), 4.93-5.02 (m, 3H), 4.52-4.62 (m, 3H), 4.12 (br t, J=4.52 Hz, 4H), 4.03 (s, 12H), 3.86-3.94 (m, 3H), 3.78-3.85 (m, 3H), 3.70-3.77 (m, 5H), 3.52-3.69 (m, 16H), 3.22 (br d, J=6.78 Hz, 2H), 2.75 (t, J=5.90 Hz, 2H), 2.10 (s, 9H), 1.99 (s, 9H), 1.89 (s, 9H), 1.76 (s, 9H), 1.47-1.58 (m, 4H), 1.33 (br s, 4H), 1.06-1.19 (m, 12H). ³¹P NMR: δ ppm 146.32.

### Example 31

Synthesis of GalNAc Cluster Conjugated FXII siRNAs

Experiments were carried out for each of Ligand A-I, with each serving as a GalNAc cluster that was conjugated to the FXII siRNA.

### Methods

### Synthesis and Purification of Sense and Antisense Strands

All sense and antisense strands were synthesized based on standard solid phase oligonucleotide synthesis technology using phosphoramidite intermediates. AKTA oligo pilot plus 10 synthesizer (GE Healthcare) was used. Synthesis was performed on a solid support made of controlled pore glass (Universal CPG, loading: 36.2 μmol/g, 1000 Å). All 2′-modified phosphoramidite were purchased from commercial sources. Specifically, the following 2′-F and 2′-O-methyl phosphoramidites were used: DMT-2′-F-Bz-dA phosphoramidite, DMT-2′-F-dU phosphoramidite, DMT-2′-F-ibu-dG phosphoramidite, DMT-2′-F-Ac-dC phosphoramidite, DMT-2′-OMe-Bz-A phosphoramidite, DMT-2′-OMe-U phosphoramidite, DMT-2′-OMe-ibu-G phosphoramidite, DMT-2′-OMe-Ac-C phosphoramidite. Amidites were dissolved in anhydrous acetonitrile (100 mM) and dried over molecular sieves (3 Å). ETT (5-ethylthio-1H-tetrazole, 600 mM in acetonitrile) was used as the activation agent. Synthesis of sense and antisense strands were carried out at 3 μmol scale. The solid phase synthesis cycle is shown in Table 1.

TABLE 1
Synthesis condition for sense and antisense
strand using 2′-modified phosphoramidites

| Step | Operation | Reagent | Time (min) |
|---|---|---|---|
| 1 | Deblocking | 3% CCl₃COOH in CH₂Cl₂ | 1 |
| 2 | Coupling | ETT 0.60M in | 5 |
| | | acetonitrile + 0.10M | |
| | | amidite in | |
| | | acetonitrile (6 eq.) | |
| 3 | Oxidation/Thiolation | Oxidation: 0.05M I₂ in | 2 |
| | | pyridine/H₂O/THF | |
| | | (2/1/7, v/v/v) | |
| | | Thiolation: PADS 0.16M in | 4 |
| | | pyridine/Acetonitrile | |
| | | (1/1, v/v) | |
| 4 | Capping | Ac₂O/THF | 1 |
| | | (10/90, v/v) | |
| | | pyridine/imidazole/THF | |
| | | (10/16/74, v/v/v) | |

Coupling of GalNAc ligand clusters to the sense strand of FXII siRNA was carried out manually in a glove box under inert atmosphere. CPG supported sense strand (3 μmol) in anhydrous Acetonitrile (3 mL) was dried over molecular sieves (3 Å) for 30 min. Ligand cluster (24 μmol, 8 eq.) in anhydrous Acetonitrile (1 mL, dried with molecular sieves (3 Å) for 30 min.) and activator (ETT, 0.5 mL, 0.6 M in Acetonitrile, dried by molecular sieves (3 Å) for 30 min.) were added. The reaction mixture was shaken for 1.5 hours at ambient temperature. Solvent was removed from CPG by syringe. The resulting CPG support resin was treated with PADS (0.16 M in pyridine/acetonitrile 1/1, v/v) at 20° C. The reaction mixture was hold at 20° C. for 20 min. CPG support was washed with acetonitrile (5 mL×4) by filtration to generate the corresponding sense strand on CPG support.

The CPG supported sense or antisense strand (3 μmol) was treated with 20% (v/v) diethylamine in acetonitrile (5 mL) for 10 min. at 20° C. The resin was washed with acetonitrile (5 mL×3) by filtration. The CPG support was treated with a 1:1 volume solution of 40% methylamine in water and 35% ammonium hydroxide solution (1.5 mL) for 10 min. at 65° C. The mixture was filtered, and the filtrate was concentrated at 40° C. with centrifugal vacuum concentrator. Crude oligonucleotide product was obtained as white solid.

Crude oligonucleotides were purified by HPLC using Durashell C18 (L) column 10×100 mm, 5 μm particle size. Mobile Phase A was 220 mM HFIP and 8.8 mM TEA in Milli Q water, pH 7.5 and mobile Phase B was methanol. The gradient was mobile phase B from 5% to 29% in 16 min. and flow rate was 3.5 mL/min. The column temperature was held at 50° C.

Annealing of sense and antisense strands and purification of siRNA The sense strand was mixed with the equimolar antisense sense strand in phosphate-buffered saline (pH7.4) to form the duplex. The temperature of annealing was set at 20° C. The concentration of oligonucleotide was 3 μmol/400 μl 1×PBS. The annealing solution was monitored by HPLC.

The duplex was purified by IP-RP HPLC using Durashell C18(L) column 10×100 mm, 5 μm particle size. Mobile Phase A was 100 mM HFIP and 20 mM HA in Milli Q water containing 5% acetonitrile and Mobile Phase B was 20% Milli Q water in acetonitrile. The gradient was mobile phase B from 18% to 35% in 18 min. and follow rate was 4 mL/min. The column temperature was set at 17° C. Fractions contain desired duplex were collected and lyophilized to afford final product.

GalNAc Conjugated FXII siRNAs

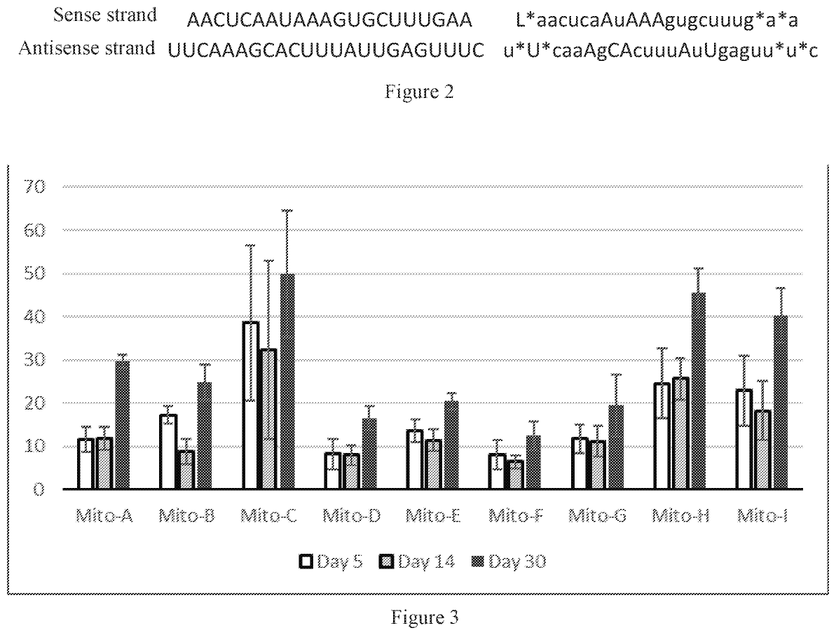

The sequence and nucleotide modification of Coagulation Factor XII (FXII) siRNA was adopted from literature (Liu et al., (2019) RNA 25, 255-263.). Sense strand: L*aacucaAuAAAgugcuuug*a*a (SEQ ID NO: 2); antisense strand: u*U*caaAgCAcuuuAuUgaguu*u*c (SEQ ID NO: 4) (from 5′ to 3′, upper case and lower case letters indicate 2-deoxy-2-fluoro (2′-F), and 2′-O-methyl (2′-OMe) ribo-sugar modifications, respectively; (*) indicates phosphorothioate linkage (PS). L indicates the Mito GalNAc ligand cluster. The representative structure of Mito GalNAc phosphoramidite used for synthesis of GalNAc conjugated FXII siRNAs is shown in FIG. 1 and information on representative GalNAc conjugated FXII siRNAs that were prepared and tested is listed in Table 2. Information on the siRNA sequences used in the studies is provided in FIG. 2.

TABLE 2
Compound information of GalNAc conjugated FXII siRNAs.
Each of the IDs corresponds to an embodiment of a targeting
ligand cluster/nucleic acid complex, in which the letter
in the ID corresonds to a Ligand (see FIG. 2), and the
siRNA is FXII siRNA as described above.

| | Calculated MS | Found MS (m/z) | |
|---|---|---|---|
| | Antisense and sense | antisense and | |
| ID | strand | sense strand | HPLC purity |
| Mito-A | 7545.93; 8318.95 | 7546.43; 8319.64 | 90.59% |
| Mito-B | 7545.93; 8318.95 | 7546.40; 8319.54 | 94.97% |
| Mito-C | 7545.93; 8304.92 | 7546.39; 8305.58 | 93.29% |
| Mito-D | 7545.93; 8320.97 | 7546.42; 8321.6 | 88.37% |
| Mito-E | 7545.93; 8308.91 | 7546.33; 8309.23 | 89.59% |
| Mito-F | 7545.93; 8352.97 | 7546.41; 8353.63 | 91.02% |
| Mito-G | 7545.93; 8186.79 | 7546.56; 8187.56 | 93.09% |
| Mito-H | 7545.93; 8220.81 | 7546.34; 8221.55 | 89.12% |
| Mito-I | 7545.93; 8188.81 | 7546.52; 8189.56 | 91.30% |

### Example 32

Testing GalNAc Conjugated FXII siRNA in Mice

### Introduction

Coagulation Factor XII (FXII) has been used as a model to assess delivery of siRNA to cells, tissues, and subjects. Experiments were conducted in which different embodiments of targeting ligand complex of the invention were conjugated to a FXII siRNA and administered in vivo. The effects of the siRNA were monitored at intervals following the administration. One means of monitoring was determining an FXII level in serum collected from mice that had been administered one of the targeting ligand complexes conjugated to an FXII siRNA.

### Methods

### Targeting Ligand Cluster/Nucleic Acid Complexes

The targeting ligand cluster/nucleic acid complexes set forth as Mito-A through Mito-I each comprises a different targeting ligand cluster conjugated to an siRNA. The targeting ligand cluster/nucleic acid complexes were referred to as Mito GalNAc conjugated FXII siRNAs. The targeting ligand clusters in this study were: Ligand A, Ligand B, Ligand C, Ligand D, Ligand E, Ligand F, Ligand G, Ligand H, and Ligand I (see FIG. 1 for structure of each). Each Mito GalNAc conjugated FXII siRNA used in the experiment comprised one of Mito-A-Mito-I conjugated to the FXII siRNA described in Example 31 herein, and are referred to herein as: Mito-A, Mito-B, Mito-C, Mito-D, Mito-E, Mito-F, Mito-G, Mito-H, and Mito-I. Further information on the complexes is provided elsewhere herein.

### In Vivo Testing

Experiments were performed to assess the effect of FXII siRNA in vivo. Male C57BL/6 mice (Jackson Labs) were subcutaneously (S.C.) administered a single dose of PBS or a Mito GalNAc conjugated FXII siRNAs at 3 mg/kg formulated in PBS (n=3 per group). A complex was prepared and tested that comprised Mito-A-Mito-I. At day 5, 14, and 30 after administration, plasma samples were collected. FXII level in plasma was evaluated using ELISA kits from Molecular Innovations following the manufacturer's instructions. The calculated plasma FXII concentrations for the Mito GalNAc conjugated FXII siRNAs (Mito-A-Mito-I) treated groups were then normalized to the average of the PBS-treated group. Structures of Ligands A-I that are included in complex Mito-A through Mito-I, respectively, are provided in FIG. 1.

TABLE 3
Data generated from treatment with: Mito-A-Mito-I and
PBS. The amounts under the Day 5, Day 14, and Day
30 columns are the percentage remaining versus the
amount remaining in PBS administered (control) mice.

| Sample ID | | | | | | |
|---|---|---|---|---|---|---|
| Compound | Day 5 | STDEV | Day 14 | STDEV | Day 30 | STDV |
| Mito-A | 30.5 | 32.8 | 11.8 | 2.8 | 29.6 | 1.5 |
| Mito-B | 17.2 | 1.9 | 8.8 | 2.8 | 24.9 | 4.0 |
| Mito-C | 38.5 | 17.9 | 32.3 | 20.6 | 49.9 | 14.8 |
| Mito-D | 8.2 | 3.6 | 7.9 | 2.3 | 16.4 | 2.8 |
| Mito-E | 13.6 | 2.7 | 11.3 | 2.5 | 20.4 | 1.9 |
| Mito-F | 8.0 | 3.4 | 6.4 | 1.5 | 12.5 | 3.3 |
| Mito-G | 11.7 | 3.3 | 11.1 | 3.5 | 19.4 | 7.1 |
| Mito-H | 24.5 | 8.2 | 25.6 | 4.8 | 45.5 | 5.7 |
| Mito-I | 22.8 | 8.1 | 18.2 | 6.8 | 40.3 | 6.2 |
| PBS | 100 | | 100 | | 100 | |

Table 3 and FIG. 3 provide data from in vivo testing. The results indicate the percent of the FXII remaining in serum collected at day 5, Day 14, and Day 30 post administration. Data was obtained following administration of each of Mito-A through Mito-I. The results showed significant reduction in FXII in plasma for all of Mito-A-Mito-I compared to the PBS level of FXII, which remained at 100%. The results of the study demonstrated the targeting ligand clusters resulted in effective in vivo delivery of the functional siRNA.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated herein in their entirety herein by reference.

What is claimed is:

1. A compound comprising a targeting ligand cluster of Formula 2 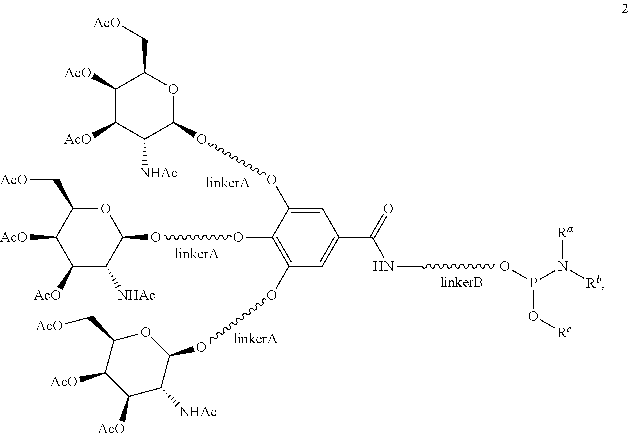
wherein linkerA is independently selected and comprises at least one spacer, with one end of linkerA attaching to a GalNAc targeting ligand and the other end attaching to a phenolic hydroxy group of gallic acid through an ether bond;
wherein linkerB is independently selected and comprises at least one spacer, with one end of linkerB attaching to a phosphorous atom of a phosphoramidite or an oligonucleotide and the other end attaching to the carboxylic acid of gallic acid through an amide bond;
wherein R^{a} comprises a C1 to C6 alkyl, C3 to C6 cycloalkyl, an isopropyl group, or R^{a} is joined with R^{b} through a nitrogen atom to form a cycle;
wherein R^{b} comprises a C1 to C6 alkyl, C3 to C6 cycloalkyl, an isopropyl group, or R^{b} is joined with R^{a} through a nitrogen atom to form a cycle; and
wherein R^{c} comprises a phosphite and phosphate protecting group, or a 2-cyanoethyl group.

2. The compound of claim 1, wherein the independently selected linkerA comprises at least one of polyethylene glycol (PEG), an alkyl group, a cycloalkyl group, an alkenyl group, a cycloalkenyl group, an alkynyl group, an aryl group, an aralkyl group, an aralkenyl group, and an aralkynyl group.

3. (canceled)

4. The compound of claim 1, wherein the independently selected linkerB comprises at least one of PEG, an alkyl group, a cycloalkyl group, an alkenyl group, a cycloalkenyl group, an alkynyl group, an aryl group, an aralkyl group, an aralkenyl group, and an aralkynyl group.

5-6. (canceled)

7. The compound of claim 1, wherein the independently selected linkerA comprises one or more of: 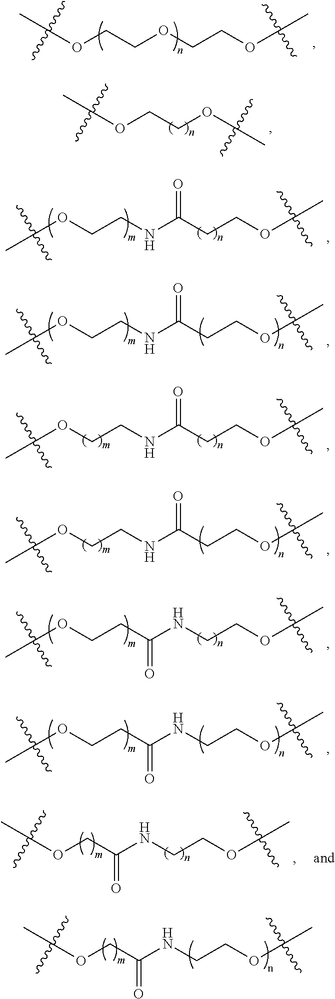
wherein m is an integral number 1, 2, 3, 4, 5, 6, 7, 8, 9, 10, 11, or 12; and n is an integral number 1, 2, 3, 4, 5, 6, 7, 8, 9, 10, 11, or 12.

8. The compound of claim 1, wherein the independently selected linkerB comprises one or more of: 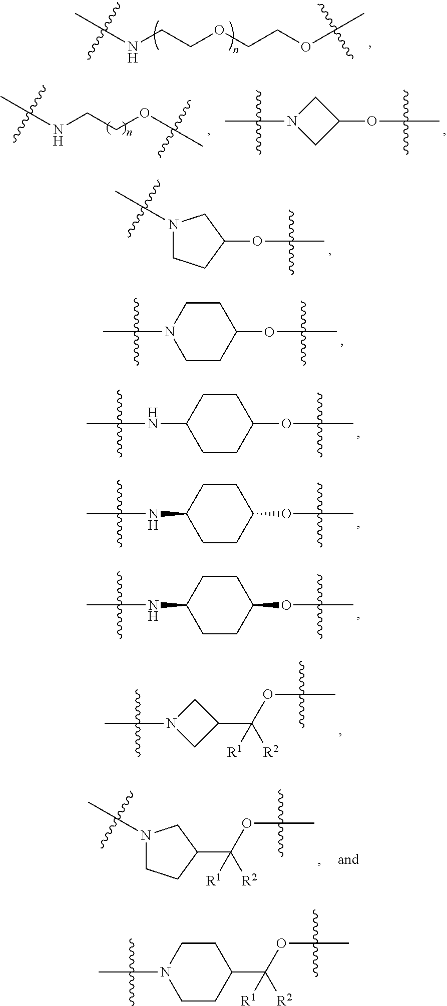
wherein n is an integral number 1, 2, 3, 4, 5, 6, 7, 8, 9, 10, 11, or 12;
wherein R¹ comprises H, methyl (Me), ethyl (Et), cyclopropyl, or R¹ is joined with R² through a carbon atom to form a 3-6 member ring; and
wherein R² comprises H, Me, Et, cyclopropyl, or R² is joined with R¹ through a carbon atom to form a 3-6 member ring.

9. (canceled)

10. The compound of claim 1, wherein the targeting ligand cluster comprises one of Ligands A-I.

11. The compound of claim 1, wherein the targeting ligand cluster comprises one of Ligands J-WW.

12. The compound of claim 1, wherein the targeting ligand cluster comprises a Gallic acid and at least one of the independently selected LinkerA comprises polyethylene glycol (PEG) directly bonded to the oxygen of a hydroxyl group of the Gallic acid.

13. The compound of claim 1, wherein the targeting ligand cluster further comprises an oligonucleotide attached to the targeting ligand cluster thereby forming a targeting ligand cluster/nucleic acid complex.

14. The compound of claim 13, wherein the targeting ligand cluster/nucleic acid complex is MITO-A, MITO-B, MITO-C, MITO-D, MITO-E, MITO-F, MITO-G, MITO-H, or MITO-I.

15. A composition comprising the compound of claim 1, optionally further comprising a pharmaceutically acceptable carrier.

16-29. (canceled)

30. A compound comprising a targeting ligand cluster of Formula 1 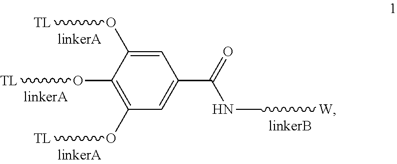
wherein TL is one or more targeting ligands, including but not limited to: N-acetylgalactosamine, galactose, galactosamine, N-formyl-galactosamine, N-propionylgalactosamine, N-n-butanoylgalactosamine, and N-iso-butanoylgalactosamine;
wherein one or more TLs may be different from one or more other TLs of the same targeting ligand cluster;
wherein linkerA is independently selected and comprises one or more bifunctional spacers, with one end of linkerA attaching to the targeting ligand and the other end attaching to a phenolic hydroxy group of gallic acid through an ether bond;
wherein linkerB is independently selected and comprises a bifunctional spacer, with one end of linkerB attaching to a phosphoramidite or an oligonucleotide and the other end attaching to the carboxylic acid of gallic acid through an amide bond; and
wherein W is H, a protecting group, phosphoramidite or oligonucleotide.

31. The compound of claim 30, wherein the targeting ligand cluster comprises one of Ligands A-I.

32. The compound of claim 30, wherein the targeting ligand cluster comprises one of Ligands J-WW.

33. The compound of claim 30, wherein the targeting ligand cluster comprises a Gallic acid; and at least one of the independently selected linkerA comprises polyethylene glycol (PEG) directly bonded to the oxygen of a hydroxyl group of the Gallic acid.

34. The compound of claim 30, wherein the targeting ligand cluster further comprises an oligonucleotide attached to the targeting ligand cluster thereby forming a targeting ligand cluster/nucleic acid complex.

35. The compound of claim 34, wherein the targeting ligand cluster/nucleic acid complex comprises a compound set forth as MITO-A, MITO-B, MITO-C, MITO-D, MITO-E, MITO-F, MITO-G, MITO-H, or MITO-I.

36. A composition comprising the compound of claim 30, optionally further comprising a pharmaceutically acceptable carrier.

37.-64. (canceled)

65. The composition comprising of claim 36, conjugated to an siRNA.

66-68. (canceled)

69. A method of reducing expression of a target gene in a cell comprising:
contacting a cell capable of expressing the target gene with a composition comprising the targeting ligand cluster of Formula 1 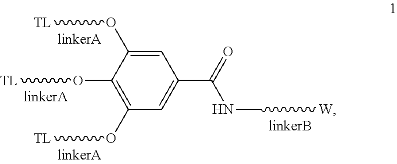
wherein TL is one or more targeting ligands, including but not limited to: N-acetylgalactosamine, galactose, galactosamine, N-formyl-galactosamine, N-propionylgalactosamine, N-n-butanoylgalactosamine, and N-iso-butanoylgalactosamine;
wherein one or more TLs may be different from one or more other TLs of the same targeting ligand cluster;
wherein linkerA is independently selected and comprises one or more bifunctional spacers, with one end of linkerA attaching to the targeting ligand and the other end attaching to a phenolic hydroxy group of gallic acid through an ether bond;
wherein linkerB is independently selected and comprises a bifunctional spacer, with one end of linkerB attaching to a phosphoramidite or an oligonucleotide and the other end attaching to the carboxylic acid of gallic acid through an amide bond; and
wherein W is an oligonucleotide comprising an siRNA that reduces expression of the target gene.

70-77. (canceled)
